(19) United States
(12) Patent Application Publication
(10) Pub. No.: US 2024/0197925 A1
(43) Pub. Date: Jun. 20, 2024

(54) FUNCTIONALIZED DIBLOCK COPOLYMER AND ITS PREPARATION METHOD AND APPLICATION

(71) Applicant: InnovatingBio (Shanghai) Co.,Ltd., Shanghai (CN)

(72) Inventors: Chun ZHOU, Shanghai (CN); Zhenxing YE, Shanghai (CN); Chenhong LU, Shanghai (CN)

(73) Assignee: InnovatingBio (Shanghai) Co.,Ltd., Shanghai (CN)

(21) Appl. No.: 18/028,218

(22) PCT Filed: Sep. 24, 2021
(86) PCT No.: PCT/CN2021/120179

§ 371 Date: Dec. 29, 2023

(30) Foreign Application Priority Data
Sep. 25, 2020 (CN) 2020110214021

(51) Int. Cl. A61K 49/00; A61K 47/69; C08G 63/91

(52) U.S. Cl. CPC A61K 49/0054; A61K 47/6937; C08G 63/912

(57) ABSTRACT

A functionalized diblock copolymer having the chemical structure shown in Formula III is provided. The functionalized diblock copolymer or polymer particles can be widely used in tumor imaging, tumor therapy and other fields. It not only has good safety, realizes faster and adjustable degradation and removal of polymers (by changing the structure and number of functional groups) under acidic conditions, but also has excellent specific and high-quality imaging effects at the target site, with high signal-to-noise ratio, clear boundaries, long half-life, etc., which solves the problem of fluorescence imaging technology in real-time intra-operative navigation, and thus has a good industrialization prospect.

### FIELD OF THE INVENTION

The present disclosure relates to the field of organic chemistry, in particular to a functionalized diblock copolymer and a preparation method and uses thereof. These uses mainly include uses for preparing tumor imaging probe reagents and uses for the manufacture of medicaments for the treatment of tumor.

### BACKGROUND OF THE INVENTION

Malignant tumors (cancers) have become one of the main reasons that threaten human lives and the threat is increasing year by year. According to the 2019 National Cancer Report issued by the National Cancer Center of China, in China, malignant tumors have become one of the main public health problems that seriously threaten the health of the Chinese population. The latest statistics show that deaths from malignant tumors accounted for 23.91% of all deaths among residents. This resulted in medical expenses exceeding 220 billion RMB. In 2015, there were approximately 3.929 million cases of malignant tumors nationwide and 2.338 million cases of deaths.

Currently, standard treatments of cancer include surgical dissection, chemo-therapy, radiation therapy, and emerging immuno-therapy. Surgical removal of solid tumor is still the most effective and recommended first line of care treatment. Usually, surgeons rely on pre-operative imaging diagnosis, intra-operative clinical experience (including visual identification and palpation, etc.), and other clinical aids to determine the boundary of the tumor, for performing resection of the lesion during the surgical operation. However, because tumors are heterogeneously distributed tissues, and different types of tumors have different boundary characteristics, it is difficult to accurately determine tumor boundaries during surgery. Excessive surgical resection may seriously affect patients' post-operative quality of life (for example, total mastectomy for breast cancer; failure to preserve healthy parathyroid glands during thyroid cancer surgery; anal preservation problems caused by surgery for low rectal cancer, etc.). Insufficient resection is prone to recurrence (for example, non-invasive bladder cancer resection surgery has a high recurrence rate due to high rate of tumor positive surgical margin). Consequently, accurately judging the boundaries of tumor lesions during surgery has become a key factor for the success of surgical operations and patient prognosis.

During the surgical procedure of tumor resection, the surgeon usually needs to decide whether to perform dissection of lymphatic tissue based on the pre-operative imaging diagnosis and the patient's pathological stage, and removes the cancerous tissue that may have metastasized into lymphatic tissue. In situations where pre-operative diagnosis is not definite, surgeons will choose to remove some of the patient's lymphatic tissues (adjacent lymph nodes), and during the operation (the patient is still under anesthesia), the pathology department will collect the specimens, perform a quick frozen pathological diagnosis, and then provide the results back to the surgeon to decide on the necessity of further dissection of lymphatic tissue, and if yes the range and extent of dissection. Generally, the entire rapid frozen pathological examination process takes about 45 minutes to several hours. During this period, the medical team and medical resources in the operating room are all on standby, and the patient is also at increased risk of infection and prolonged anesthesia time while waiting in the operating room. Therefore, in addition to judging the boundary of the tumor, there is also a clinical need for a faster and more accurate pathological judgment of the tumor spreading tissue during the operation which shortens the operation time, accurately removes the cancer spreading tissue, reduces recurrence rate, and prolongs the patient survival after surgery.

In summary, the intra-operative imaging technology for solid tumors and metastatic tissues has great clinical significance. However, there are still great challenges for intra-operative specific imaging of cancer tissues. The main difficulties and corresponding current clinical development strategies are as follows.

1) The hardware should meet the requirements of the operating room.

Currently, widely used clinical imaging techniques such as X-ray scanning, CT (computed tomography), MRI (magnetic resonance imaging), ultrasound and PET-CT (positron emission computed tomography) are mainly used in pre-operative tumor imaging diagnosis, but less for intra-operative tumor imaging diagnosis, due to the hardware requirements (such as volume), application requirements (such as electromagnetic fields) and many other reasons, which limit the real-time imaging diagnosis of these imaging technologies on the operating table and during the operation. In the prior art, because the intra-operative ultrasound imaging technology requires contact for imaging, its application in open tumor surgery is limited, and the imaging technology itself is based on tissue morphology with high false negatives and false positives. In brain tumor surgery, MRI scanning before surgery and constructing surgery coordinate information are also clinically applied during surgery, but this technology may affect the navigation quality of the surgery due to the deformation or displacement of the tissue from the time of image acquisition to the period of surgery.

Compared with the above-mentioned imaging technology, the technology based on fluorescence imaging has advantages in real-time application of surgery. First of all, the near-infrared (NIR) light source commonly used in fluorescence imaging technology has a stronger penetrating ability in tissues than visible light, ultraviolet light and other light sources, and is less affected by the main absorption chromophores inside the tissue, such as hemoglobin, oxygenated hemoglobin, and water. It can penetrate about 1 cm of tissue, and it has very important application value in tissue optical inspection, especially for shallow tissues. Secondly, the hardware implementation of fluorescence imaging can be more flexible. It can be designed as a movable imaging system integrating light sources of visible (white) light and NIR. Such imaging system can be configured for open surgery, or it can be designed as a small sterile probe with an external display screen to achieve white light and fluorescence endoscopic imaging system for minimally invasive surgery in the body. These two hardware designs have been approved by FDA and EMA (e.g., SPY Imaging system; PINPOINT® endoscopic fluorescence imaging system; da Vinci surgical robot system), and have been successfully applied in clinical surgery. Using a fluorescence microscope system, within 20 minutes after intra-operative intravenous injection of indocyanine green (ICG), ICG can be used to excite fluorescence under near-infrared light source for angiography (neurosurgery, vascular surgery, eye surgery, etc.). Methylene blue is also an approved fluorescent imaging agent and is used in some surgical procedures.

2) The intra-operative imaging technology should be specific to the tumor tissue.

The main requirements for achieving tumor specificity are: first of all, the targeted tumor must have some specific characteristics. Some of the characteristics that are currently widely recognized are: specific surface receptors (such as folic acid, Her2/Neu, EGFR, PSMA and other receptors); characteristics of tumor microenvironment (specific metabolites, proteases; or acidic characteristics inside cancer cells (pHi: 5.0-6.0) or in the interstitial fluid between cells (pHe: 6.4-6.9), which are originated from the lactic acid metabolites produced by the cancer cells after the rapid ingestion of glucose). Secondly, the above-mentioned specific characteristics should be used as a precise positioning target for the developed imaging technology, so as to effectively realize the specific accumulation of imaging agents at the tumor site. The usual means to achieve the accumulation of imaging agents at tumor sites are: using the specific receptors of cancer cells to achieve the specific binding of the imaging agent to them; using the acidity or other characteristics of the tumor microenvironment to retain and enrich the imaging agents at tumor site by chemical means; and using the enhanced permeability and retention effect (EPR) of tumor tissues to achieve selective local accumulation of some nanoparticles.

3) The imaging agent must be safe and can be degraded or eliminated from the body within a short period of time after use. There should be low tissue residue and no side effects. If a metabolic reaction occurs, the metabolites of the imaging agent should be harmless to human body.

The main clinical translation of intra-operative imaging technology for solid tumors uses the following types of technical means.

1) Folic Acid-Florescence Dye conjugate: On Target Laboratories has conducted a few clinical trials for conducting intra-operative tumor imaging guided surgeries for lung cancer and ovarian cancer. The advantage is that for these two selected tumors, the target selection strategy is clear (except for individual tissues, folate receptors are expressed at very low levels on normal tissues, and overexpressed on the surface of some tumor cells). The disadvantage is that the application area is relatively narrow (only applicable to specific tumors with high folate receptor expression), and from the perspective of its imaging principles and clinical data, the quality of tumor specific imaging is somewhat limited (background contrast; the boundary between tumor and healthy tissue is not clearly distinguished), and the reason may be that imaging molecules that normally circulate in the body (not bound to tumor receptors) can fluoresce when illuminated by the excitation light source, causing background fluorescence, or false positive images of non-tumor sites (“off-target” phenomenon, for example, in some healthy tissues such as kidneys, there are also different degrees of folate receptors), or may be that the expression of folic acid in tumor tissues may not be completely uniform due to the heterogeneity of tumors mentioned above, causing defects in image quality. From the clinical data, the clearance of the background is related to the dosage. Basically, it takes 24 hours to 4 days for complete clearance. The effect of tumor imaging (tumor/normal tissue ratio, abbreviated as TNR, value of 2-3) is acceptable but improvement could be desired.

2) Antibody (mAB)-Florescence Dye conjugate: there have been several clinical trials for intro-operative imaging navigation of tumors such as glioma (mAB targeting EGFR receptors, Cetuximab) and colon cancer, and lung cancer (targeting CEA receptor). Compared with folic acid-florescence imaging molecule conjugate design, the antibody molecule used for targeting has good biocompatibility, and the circulation cycle in the body is very long (3-7 days). For the selected tumor, the target is clear and the binding mechanism is clear. Its shortcomings are also obvious. The long circulation time of antibody molecules will also cause high background fluorescence. It also has other problems, such as narrow application range (only applicable to tumors with high expression of specific receptors). For example, there are false positive images of non-tumor sites (the selected target may exist in healthy tissues), and the non-uniform characteristics of the tumor mentioned above. From the clinical and animal research data, the tumor imaging effect of this technology (cancer/normal tissue ratio, TNR, 2-5) is acceptable, but the image is usually accompanied by strong background fluorescence.

3) Peptide-Florescence Dye conjugate: in view of the characteristics of several tumor cells and tumor microenvironment mentioned above, polypeptides can be used for selective targeting to target fluorescent imaging molecules to tumor sites. At present, there are several designs in the direction of research and development and clinical transformation: R. Tsien and Avelas Biosciences, Inc. use a special U-shaped polypeptide combination design. One end of the polypeptide is positively charged under physiological conditions (this end of the polypeptide is linked to a fluorescent imaging molecule), the other end of the polypeptide is negatively charged under physiological conditions, and the two ends of the polypeptide are connected by a linker, which can be cleaved by the protease present in the tumor microenvironment. After the disconnection, the polypeptide with the fluorescent imaging molecule exhibits a positive charge, which can be attracted to the negative charge on the surface of the cancer cell and then adsorbed on the surface. Later, it enters the cancer cell through the endocytosis mechanism, and then the imaging agent molecule that enters the cancer cell can emit fluorescence under the illumination of the excitation light source. It can be seen that after this type of imaging agent enters the body, the time window for completing this series of action within a limited time (even with PEG modification, the circulation half-life is only around 20 minutes) is not sufficient, resulting in poor imaging results (the TNR is 2-3). The team of Donald M. Engelman of Yale University proposed a different design to form a conjugate between a fluorescent molecule and a polypeptide. The signal targeted by the polypeptide is the acidic characteristic of the tumor microenvironment. Under normal physiological conditions, the polypeptide is negatively charged, but it becomes neutral in an acidic environment. Under electrically neutral conditions, the lipophilicity of the polypeptide increases, which drives the deposition and transmembrane behavior of the polypeptide on the surface of cancer cells to achieve the specific enrichment of fluorescent molecules at the tumor site. From the results of live imaging, this technology has achieved good tumor imaging quality (TNR is about 6), but the error range of the reported data is too large and the effect is not good. Lumicell's design is to connect a fluorescent imaging molecule and another molecule that can absorb fluorescence through a peptide. The selected peptide can be cleaved under the catalysis of some common proteases (such as Cathepsin K, L, S) in the tumor microenvironment, so that the fluorescent molecules and the molecules that actively absorb fluorescence will be separated and then fluoresce in the presence of the excitation light source. This design can reduce the background fluorescence during the cycle, because the entire imaging agent molecule does not fluoresce before it reaches the tumor microenvironment. By conjugating a strand of polyethylene glycol (PEG), the blood circulation time can be achieved to about 24 hours, and the tumor image quality (TNR is 3-5) is only acceptable. In addition, another disadvantage of this technology is whether the selected peptide sequence can achieve high specific tumor targeting.

4) Dye-carrying nano-particles (NP): in the field of medical imaging, nanoparticles are widely used. The main categories are liposomal nanoparticles, inorganic nanoparticles, and polymer nanoparticles. DEFINITY® is a phospholipid liposome of Lantheus Medical (now BMS) approved in 2001, and is used to stabilize perfluoropropane (C3F8) bubbles and used as an ultrasound imaging agent. There are many types of inorganic nanoparticles (silica; iron oxide; quantum dots; carbon nanotubes, etc.). Generally, the clinical application difficulty of inorganic nanoparticles is safety. And it is often difficult to achieve specific tumor fluorescence imaging if fluorescent groups are introduced merely by chemical modification on the surface of nanoparticles. U. Wiesner and others have successfully advanced several early clinical studies. The use of small particle size (5-20 nm) SiO₂ nanoparticles allows the used nanoparticles to be removed from the kidney to improve safety, and the core of the nanoparticles is embedded with fluorescent molecules, and the introduction of specific targeting groups on the surface of nanoparticles can achieve specific tumor fluorescence imaging. The fluorescent molecules introduced by this method can overcome the possible defects of fluorescence quenching of conventional nanoparticle-fluorescent molecule conjugates that may occur due to long residence time in the body, but the reported half-life is short (10-30 minutes). The tumor imaging effect is acceptable, and its TNR is 5-10 (the reported data has a large error range), but the liver absorption is also very high (the tumor/liver ratio is about 2). The author believes that although small-sized nanoparticles (less than 20nm) can be eliminated by the kidneys, they still do not rule out their clinical risks (such as spreading to the brain through the BBB, etc.). The typical structure of polymer nanoparticles is to use amphiphilic diblock polymers, such as PEG-PLGA, PEG-PEG-Glutamate, and PEG-Aspartate, which are several types of clearable (PEG)/degradable (another block) polymers that are currently working to the clinic. Building on the previous work of Langer et al. on pH-responsive polymer microspheres (the polymer backbone contains amino groups that can be protonated at pH 6.5), Kim et al. introduced PEG blocks to construct pH-responsive amphiphilic diblock copolymer. The diblock copolymer realizes the dissolution of nanoparticles in the weakly acidic environment of the tumor (the nanoparticle core is ionized in the acidic environment, and the charge repulsive force is generated, which destroys the energy balance of the amphiphilic self-assembly).

### SUMMARY

The present disclosure provides a functionalized diblock copolymer and a preparation method and uses thereof.

An aspect of the present disclosure provides a functionalized diblock copolymer. The chemical structure of the functionalized diblock copolymer is shown in Formula III:

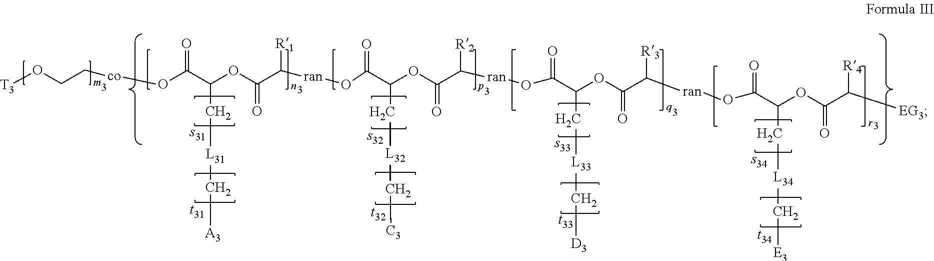

In Formula III, m₃=22˜1136, n₃=10˜500, p₃=0.5˜50, q₃=0˜500, r₃=0˜200;
s₃₁=1˜10, s₃₂=1˜10, s₃₃=1˜10, s₃₄=1˜10;
L₃₁, L₃₂, L₃₃ and L₃₄ are linking groups;
R′₁, R′₂, R′₃ and R′₄ are independently selected from H, C1-C20 alkyl and C3-C10 cycloalkyl;
A₃ is selected from protonatable groups;
C₃ is selected from fluorescent molecular groups;
D₃ is selected from delivery molecular groups;
E₃ is selected from hydrophilic/hydrophobic groups;
T₃ is selected from capping groups; and
EG₃ is selected from capping groups.

In formula II, -co- denotes block copolymers, -ran- denotes random distribution of units with different side chains within the polymer block separated by -co-. Another aspect of the present disclosure provides polymer particles prepared from the above-mentioned functionalized diblock copolymer.

Another aspect of the present disclosure provides the use of the aforementioned functionalized diblock copolymer or the aforementioned polymer particles in the preparation of imaging probe reagents and pharmaceutical preparations.

Another aspect of the present disclosure provides a composition including the aforementioned functionalized diblock copolymer or the aforementioned polymer particles.

### BRIEF DESCRIPTION OF THE DRAWINGS

FIG. 1a shows the fluorescence intensity of the polymer (IB015-038-01) nano-particle imaging probe solution of Example 6.1.5 measured at different solution pH; FIG. 1b shows the corresponding normalized fluorescence intensity against the solution pH.

FIG. 2a shows the fluorescence intensity of the polymer (IB015-055-01) nano-particle imaging probe solution of Example 11.1.5 measured at different solution pH; FIG. 2b shows the corresponding normalized fluorescence intensity against the solution pH.

FIG. 3 is a graph combining the fluorescence intensity measured by the polymer (IB015-055-01) nano-particle imaging probe solution of Example 11.1.5 at different solution pH with the fluorescence intensity measured by adding the nano-particle imaging probe solution into solvent DMF.

FIG. 4 shows the fluorescence intensity of the polymer (IB015-050-01) nano-particle imaging probe solution of Example 10.1.5 at different solution pH.

FIG. 5a shows the fluorescence intensity of polymer IB015-059-01 (Example 11.2.5) nano-particle imaging probe solution measured at different pH (aqueous solution); FIG. 5b shows the fluorescence intensity of polymer IB015-059-01 (Example 11.2.5) nano-particle imaging probe solution measured at different pH (in aqueous solution, DMF and EtOH); FIG. 5c shows that blue shift of the fluorescence emission peak occurs in the nano-particle imaging probe solution of polymer IB015-059-01 (Example 11.2.5) (normalized for fluorescence intensity).

FIG. 6 shows 24-hour in vivo imaging photographs of a 4T1 subcutaneous model Balb/c tumor-bearing mouse after injection of Example 11.1.5 (IB015-055-01) nano-particle fluorescent imaging probe, as well as fluorescent imaging photographs of harvested organs and lymph nodes.

FIG. 7 shows the fluorescence quantitative intensity values for the harvested organs of FIG. 6.

FIG. 8 shows 24-hour in vivo imaging photographs of a 4T1 subcutaneous model Balb/c tumor-bearing mouse after injection of Example 6.1.5 (IB015-038-01) nano-particle fluorescent imaging probe, as well as fluorescent imaging photographs of harvested organs and lymph nodes.

FIG. 9 shows the fluorescence quantitative intensity values for the harvested organs of FIG. 8.

FIG. 10 shows fluorescent imaging photographs (24 hours post-injection) of harvested organs and lymph nodes of a 4T1 subcutaneous model Balb/c tumor-bearing mouse after injection of Example 10.1.5 (IB015-050-01) nano-particle fluorescent imaging probe.

FIG. 11 shows the fluorescence quantitative intensity values for the harvested organs of FIG. 10.

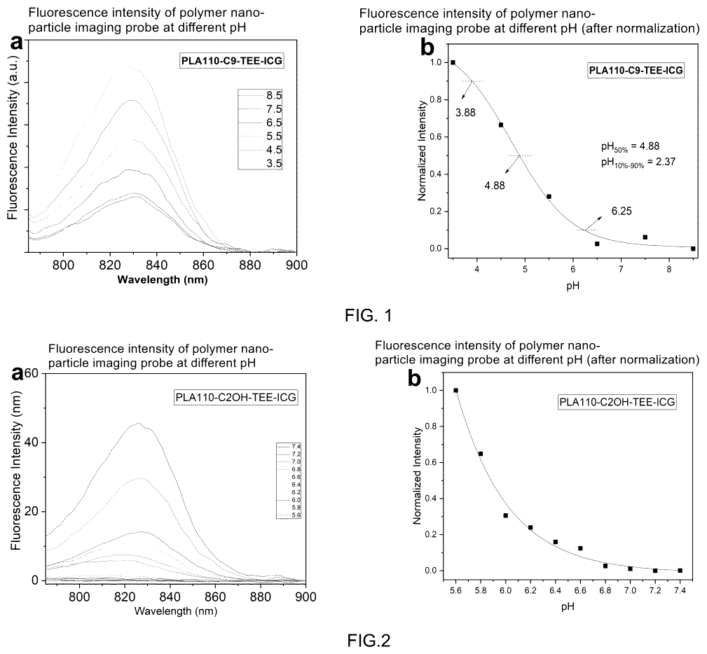

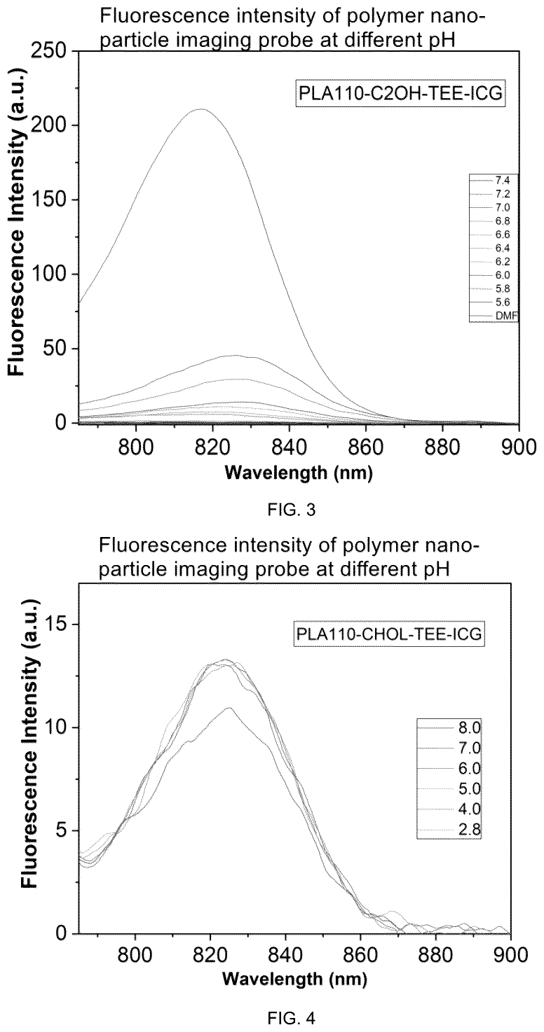

### DETAILED DESCRIPTION

In order to make the purpose of the present disclosure, technical solutions and beneficial technical effects of the present disclosure clearer, the invention will be further described in detail below in conjunction with examples. Those skilled in the art can easily understand other advantages and effects of this invention from the content disclosed in this specification.

In the present disclosure, “diblock copolymer” generally refers to a polymer having two different polymer segments (as if two blocks linked together) with different chemical compositions.

In the present disclosure, the “protonatable group” generally refers to a group that can combine with a proton, that is, it can bind at least one proton. These groups usually have a lone pair of electrons, so that at least one proton can be combined with the protonatable group.

In the present disclosure, “degradability regulating group” is a type of group that can change the degradability of a compound in vivo.

In the present disclosure, “fluorescent molecular group” generally refers to a type of group corresponding to fluorescent molecules. Compounds containing these groups can usually have characteristic fluorescence in the ultraviolet-visible-near infrared regions, and their fluorescent properties (excitation and emission wavelengths, intensity, lifetime, polarization, etc.) can change with the nature of the environment.

In the present disclosure, “delivery molecular group” usually means various molecules that can be chemically bonded to the main chain of the block copolymer through a side chain, or interact with the hydrophobic side chain groups of the block copolymer through physical force (such as charge forces, hydrogen bonding, van der Waals force, hydrophobic interaction, etc.) and can be delivered by nanoparticles formed by self-assembly of the block polymer in aqueous solution.

In the present disclosure, “hydrophilic/hydrophobic group” generally refers to a group with a certain degree of hydrophilicity or lipophilicity. In the present disclosure, “alkyl” usually refers to a saturated aliphatic group, which can be linear or branched. For example, C1-C20 alkyl usually refers to alkyl groups with 1, 2, 3, 4, 5, 6, 7, 8, 9, 10, 11, 12, 13, 14, 15, 16, 17, 18, 19, or 20 carbon atom(s). Specific alkyl groups can include, but are not limited to, methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl.

In the present disclosure, “alkenyl” generally refers to an unsaturated aliphatic group with C═C bond(s) (carbon-carbon double bonds, ethylenic bonds), which can be straight or branched. For example, C2-C10 alkenyl generally refers to alkenyl groups of 2, 3, 4, 5, 6, 7, 8, 9, or 10 carbon atoms. Specific alkenyl groups may include, but are not limited to, vinyl, propenyl, butenyl, pentenyl, hexenyl, heptenyl, octenyl, nonenyl, and decenyl.

In the present disclosure, “alkynyl” generally refers to an unsaturated aliphatic group with C≡C bond (s) (carbon-carbon triple bonds, acetylene bonds), which can be straight or branched. For example, C2-C10 alkynyl generally refers to alkynyl groups of 2, 3, 4, 5, 6, 7, 8, 9, or 10 carbon atoms. Specific alkynyl groups may include, but are not limited to, ethynyl, propynyl, butynyl, pentynyl, hexynyl, heptynyl, octynyl, nonynyl, and decynyl.

In the present disclosure, “cycloalkyl” generally refers to saturated and unsaturated (but not aromatic) cyclic hydrocarbons. For example, C3-C10 cycloalkyl generally refers to cycloalkyl groups of 3, 4, 5, 6, 7, 8, 9, or 10 carbon atoms. Specific cycloalkyl groups may include, but are not limited to, cyclopropyl, cyclobutyl, cyclopentyl, cyclohexyl, cycloheptyl, cyclooctyl, cyclononyl, cyclodecyl. The term “cycloalkyl” in the present disclosure also includes saturated cycloalkyls in which optionally at least one carbon atom can be replaced by a heteroatom, which can be selected from S, N, P, and O. In addition, a monounsaturated or polyunsaturated (preferably monounsaturated) cycloalkyl group without heteroatoms in the ring should belong to the term cycloalkyl group as long as it is not an aromatic system.

In the present disclosure, “aromatic group” generally refers to a ring system with at least one aromatic ring and no heteroatoms. The aromatic group may be substituted or unsubstituted. The specific substituent may be selected from C1-C6 alkyl, C1-C6 alkoxy, C3-C10 cycloalkyl, hydroxyl, halogen, etc. Specific aromatic groups may include, but are not limited to, phenyl, phenol, aniline, and the like.

In the present disclosure, “heteroaryl” generally refers to a ring system having at least one aromatic ring and optionally one or more (for example, 1, 2, or 3) heteroatoms selected from nitrogen, oxygen, and sulfur. The heteroaryl group may be substituted or unsubstituted, and the specific substituent may be selected from C1-C6 alkyl, C1-C6 alkoxy, C3-C10 cycloalkyl, hydroxyl, halogen and the like. Specific heteroaryl groups may include, but are not limited to, furan, benzofuran, thiophene, benzothiophene, pyrrole, pyridine, pyrimidine, pyridazine, pyrazine, quinoline, isoquinoline, phthalazine, benzo-1,2,5-thiadiazole, benzothiazole, indole, benzotriazole, benzodioxolane, benzodioxane, benzimidazole, carbazole, or quinazoline.

In the present disclosure, “targeting agents” generally refer to agents that can specifically direct a specific compound, typically via binding to specific cell receptors, to a desired site of action (target area). Targeting agents may be deployed on surface of polymeric particles as carriers and typically have high affinity to target site and relatively low, no, or almost no interaction with non-target tissues.

In the present disclosure, “imaging probe” generally refers to a class of substances that can enhance the effect of image observation after being injected (or administered) into human tissues or organs.

In the present disclosure, “individual” generally includes humans and non-human animals, such as mammals, dogs, cats, horses, sheep, pigs, cows, and the like.

After a lot of practical research, the inventors of the present disclosure have provided a class of functionalized diblock copolymers. These diblock copolymers can be pH-responsive and degradable under corresponding pH conditions through innovative chemical modification strategies. Therefore, they can be used in various fields utilizing such said features, and the present disclosure has been completed on this basis.

The first aspect of the present disclosure provides a functionalized diblock copolymer having the chemical structural formula shown below:

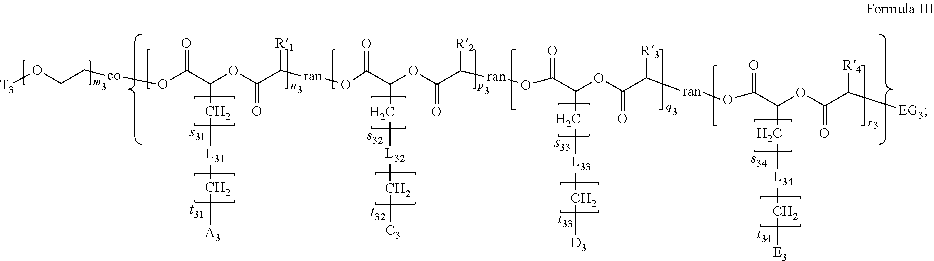

In Formula III, m₃=22˜1136, n₃=10˜500, p₃=0.5˜50, q₃=0˜500, r₃=0˜200;
s₃₁=1˜10, s₃₂=1˜10, s₃₃=1˜10, s₃₄=1˜10;
t₃₁=1˜10, t₃₂=1˜10, t₃₃=1˜10, t₃₄=1˜10;
L₃₁, L₃₂, L₃₃ and L₃₄ are linking groups;
R′₁, R′₂, R′₃ and R′₄ are independently selected from H, C1-C20 alkyl and C3-C10 cycloalkyl;
A₃ is selected from protonatable groups;
C₃ is selected from fluorescent molecular groups;
D₃ is selected from delivery molecular groups;
E³ is selected from hydrophilic/hydrophobic groups;
T₃ is selected from capping groups; and
EG₃ is selected from capping groups.

The compound of Formula III is a diblock copolymer of polyethylene glycol-polylactide, wherein the side chain structure of the polylactide block is randomly distributed, and the general formula is represented by ran.

In the compound of Formula III, L₃₁, L₃₂, L₃₃, L₃₄ are usually linking groups, which is mainly used to link the main chain of the functionalized diblock copolymer and its pendant side chains. In a specific example of the present disclosure, L₃₁, L₃₂, L₃₃, L₃₄ can be independently selected from S, O, N, —OC(O)—, —C(O)O—, SC(O)—, —C(O)—, —OC(S)—, —C(S)O—, —SS, C(R₁)═N—, —N═C(R₂), C(R₃)═N—O, —O—N═C(R₄), —N(R₅)C(O)—, —C(O)N(R₆), —N(R₇)C(S), C(S)N(R₈)—, —N(R₉)C(O)N(R₁₀), —OS(O)O—, —OP(O)O—, —OP(O)N—, —NP(O)O—, and —NP(O)N—, and wherein R₁˜R₁₀ are each independently selected from H, C1-C10 alkyl, and C3-C10 cycloalkyl.

In another specific embodiment of the present disclosure, L₃₁, L₃₂, L₃₃, and L₃₄ may be independently S.

In the compound of Formula III, A₃ is usually selected from protonatable groups, and this group and the block of the polymer in which this group is located are mainly used to adjust the pH response of the polymer. In an embodiment of the present disclosure, A₃ can be

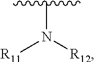

wherein, R₁₁ and R₁₂ are each independently selected from C1-C10 alkyl, C2-C10 alkenyl, C2-C10 alkynyl, C3-C10 cycloalkyl, and aryl. In another embodiment of the present disclosure, A₃ can be

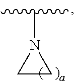

wherein, a=1-10, and a is a positive integer.

In another embodiment of the present disclosure, A₃ can be

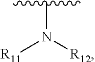

wherein R₁₁ is ethyl, R₁₂ is n-propyl. In another embodiment of the present disclosure, A₃ can be

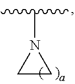

wherein, a=1-10, and a is a positive integer.

In another embodiment of the present disclosure, A₃ can be

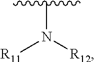

wherein R₁₁ is ethyl, R₁₂ is ethyl.

In another embodiment of the present disclosure, A₃ can be

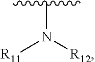

wherein R₁₁ is n-propyl, R₁₂ is n-propyl.

In another embodiment of the present disclosure, A3 can be

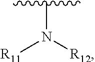

wherein R₁₁ is n-propyl, R₁₂ is n-butyl.

In another embodiment of the present disclosure, A3 can be

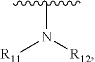

wherein R₁₁ is n-butyl, R₁₂ is n-butyl.

In the compound of Formula III, C3 is usually a fluorescent molecular group, and this group and the block of the polymer in which this group is located are mainly used to introduce fluorescent molecular groups. The fluorescent molecular group may specifically include, but is not limited to, one or a combination of organic reagents, metal chelate and the like. In an embodiment of the present disclosure, C₃ may include fluorescent molecules such as ICG (Indocyanine Green), METHYLENE BLUE, CY3, CY3.5, CY5, CY5.5, CY7, CY7.5, BDY630, BDY650, BDY-TMR, Tracy 645, and Tracy 652.

In another embodiment of the present disclosure, C3 may include indocyanine green (ICG), and ICG may be connected to the side chain of the block through an amide bond.

In the compound of Formula III, D3 can be a delivery molecular group, and this group and the block of the polymer in which this group is located are mainly used to introduce various molecular groups that can be delivered through a block copolymer. These molecular groups may include, but are not limited to, fluorescence quenching groups, drug molecule groups (for example, photodynamic therapy precursor molecules, chemotherapeutic drug molecules, biopharmaceutical molecules, etc.) and the like. In an embodiment of the present disclosure, the fluorescence quenching group can be selected from BHQ-0, BHQ-1, BHQ-2, BHQ-3, BHQ-10, QXL-670, QXL-610, QXL-570, QXL_{520,} QXL-490, QSY35, QSY7, QSY21, QXL_{680,} Iowa Black RQ, and Iowa Black FQ. In an embodiment of the present disclosure, the drug molecule group can be a group corresponding to chemotherapeutic drugs, which can specifically be nucleic acid drugs, paclitaxel, cisplatin, doxorubicin, irinotecan, SN38 and other drug molecules. In another embodiment of the present disclosure, the drug molecule group can be a group corresponding to photodynamic therapy chemical drugs, and may specifically be a group corresponding to 5-ALA and its derivative structure (lipidation or fatty chaining, etc.). The specific chemical structure of the group is as follows:

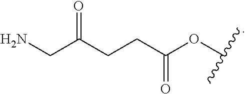 [SMILES: *OC(=O)CCC(=O)CN]

In the compound of Formula III, E₃ can be a hydrophilic/hydrophobic group, and this group and the block of the polymer in which this group is located are mainly used to adjust the hydrophobicity/hydrophilicity of the hydrophobic block of the polymer. In a specific embodiment of the present disclosure, E₃ can be selected from H, C1-C18 alkyl, —O—R₁₁, —S—R₁₂, wherein R₁₁˜R₁₂ are each independently selected from H, C1-C18 alkyl, C3-C10 cycloalkyl, aryl, and heteroaryl. Preferably, E₃ can be selected from —(CH₂—CH₂—O)n-H (n=1˜30), —(R₁₄)—NH₂ where R₁₄ is repeated methylene group (—CH₂-)n (n=1˜18), —(R₁₅)—OH where R₁₅ is repeated methylene group (—CH₂-)n (n=1˜18), sugar groups such as monosaccharides, disaccharides. Preferrably, E₃ can be selected from cholesterol and its' derivatives, hydrophobic vitamins such as Vitamin E and Vitamin D, and zwitterionic groups (with exemplar structures shown below). All above-mentioned hydrophilic or hydrophobic groups can either be used alone as an E₃ group, or be used in combination as an E₃ group. When hydrophilic and hydrophobic groups are used simultaneously, the total number of all hydrophilic and hydrophobic groups can be described as r₃ where r_{3,A} is the total number of hydrophilic groups, and r_{3,B} is the total number of hydrophobic groups.

The chemical structural formula of the cholesterol or cholesterol derivative, vitamin D, or vitamin E may be one of those shown below:

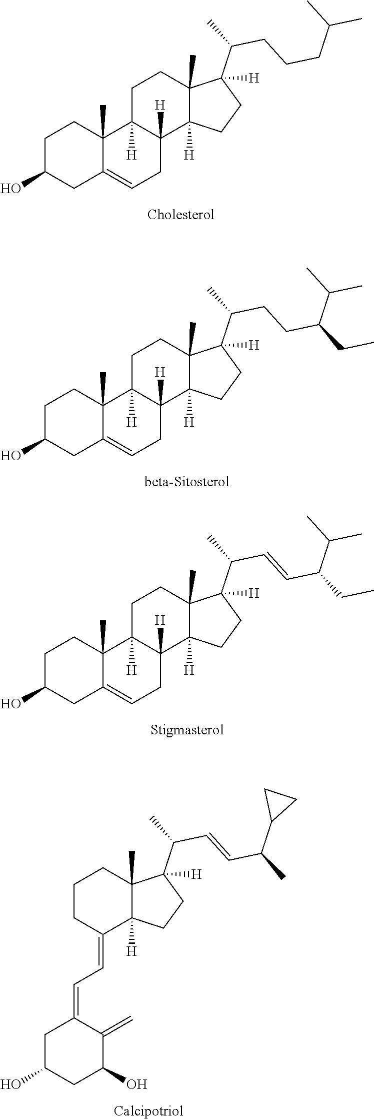

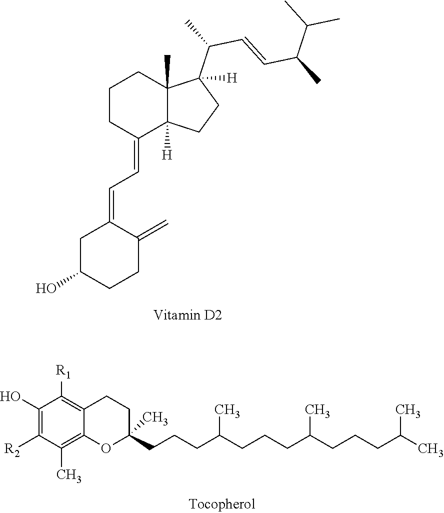

The chemical structural formula of the zwitterionic group may be one of the following:

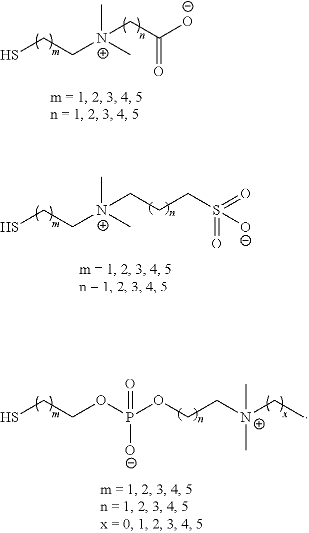

In an embodiment of the present disclosure, E₃ may be n-nonyl.

In another embodiment of the present disclosure, E₃ may be n-octyl.

In another embodiment of the present disclosure, E₃ may be n-butyl.

In an embodiment of the present disclosure, E₃ may be n-propyl.

In another embodiment of the present disclosure, E₃ may be ethyl.

In another embodiment of the present disclosure, E₃ may be methyl.

In another embodiment of the present disclosure, E₃ may be n-octadecyl.

In another embodiment of the present disclosure, E₃ may be n-heptadecyl.

In another embodiment of the present disclosure, E₃ may be cholesterol.

In another embodiment of the present disclosure, E₃ may a cholesterol derivative.

In another embodiment of the present disclosure, E₃ may be hydroxyethyl.

In another embodiment of the present disclosure, E₃ may be hydroxymethyl.

In another embodiment of the present disclosure, E₃ may be hydroxypropyl.

In another embodiment of the present disclosure, E₃ may be hydroxybutyl.

In another embodiment of the present disclosure, E₃ may be a zwitterionic group.

In an embodiment of the present disclosure, E₃ may include a zwitterionic group and n-nonyl.

In another embodiment of the present disclosure, E₃ may include a zwitterionic group and n-octyl.

In the compound of Formula III, T3 can usually be selected from end groups of polyethylene glycol (PEG) initiators. In an embodiment of the present disclosure, T₃ can be selected from —CH₃ and H.

In the compound of formula III, EG₃ can usually be produced by different capping agents added after polymerization. In a specific embodiment of the present disclosure, EG₃ can be —Y—R₁₃, wherein Y is selected from O, S, and N, and R₁₃ is selected from H, C1-C20 alkyl, C3-C10 cycloalkyl, and aryl.

In another embodiment of the present disclosure, EG₃ can be —OH.

In the compound of Formula III, the molecular weight of polyethylene glycol (PEG) block can be in a range of 1000˜50000 Da, 1000˜2000 Da, 2000˜3000 Da, 3000˜4000 Da, 4000˜5000 Da, 5000˜6000 Da, 6000˜7000 Da, 7000˜8000 Da, 8000˜9000 Da, 9000˜10000 Da, 10000˜12000 Da, 12000˜14000 Da, 14000˜16000 Da, 16000˜18000 Da, 18000˜20000 Da, 22000˜24000 Da, 24000˜26000 Da, 26000˜28000 Da, 28000˜30000 Da, 30000˜32000 Da, 32000˜34000 Da, 34000˜36000 Da, 36000˜38000 Da, 38000˜40000 Da, 40000˜42000 Da, 42000˜44000 Da, 44000˜46000 Da, 46000˜48000 Da, or 48000˜50000 Da; the molecular weight of polylactide block can be in a range of 5000˜50000 Da, 5000˜6000 Da, 6000˜7000 Da, 7000˜8000 Da, 8000˜9000 Da, 9000˜10000 Da, 10000˜12000 Da, 12000˜14000 Da, 14000˜16000 Da, 16000˜18000 Da, 18000˜20000 Da, 22000˜24000 Da, 24000˜26000 Da, 26000˜28000 Da, 28000˜30000 Da, 30000˜32000 Da, 32000˜34000 Da, 34000˜36000 Da, 36000˜38000 Da, 38000˜40000 Da, 40000˜42000 Da, 42000˜44000 Da, 44000˜46000 Da, 46000˜48000 Da, 48000˜50000 Da, 52500˜55000 Da, 57500˜60000 Da, 60000˜62500 Da, 62500˜65000 Da, 67500˜70000 Da, 72500˜75000 Da, 77500˜80000 Da, 82500˜85000 Da, 85000˜87500 Da, 87500˜90000 Da, 90000˜92500 Da, 92500˜95000 Da, 95000˜97500 Da, 97500˜100000 Da, 100000˜102500 Da, 102500˜105000 Da, 105000˜107500 Da, 107500˜110000 Da, 110000˜112500 Da, 112500˜115000 Da, 115000˜117500 Da, 117500˜120000 Da, 120000˜122500 Da, 122500˜125000 Da, 125000˜127500 Da, or 127500˜130000 Da.

In a specific embodiment, the molecular weight of the polyethylene glycol block can be in a range of 2000˜10000 Da, and the molecular weight of the polylactide block can be in a range of 4000˜26000 Da, 20000˜40000 Da, or 40000˜60000 Da.

In the compound of Formula III, m₃ can be in a range of 22˜1136, 22˜32, 32˜42, 42˜52, 52˜62, 62˜72, 72˜82, 82˜92, 92˜102, 102˜122, 122˜142, 142˜162, 162˜182, 182˜202, 202˜242, 242˜282, 282˜322, 322˜362, 362˜402, 402˜442, 442˜482, 482˜522, 522˜562, 562˜602, 602˜642, 642˜682, 682˜722, 722˜762, 762˜802, 802˜842, 842˜882, 882˜902, 902˜942, 942˜982, or 982˜1136.

n₃ can be in a range of 10˜500, 10˜15, 15˜20, 20˜25, 25˜30, 30˜35, 35˜40, 40˜45, 45˜50, 45˜50, 50˜60, 60˜70, 70˜80, 80˜90, 90˜100, 100˜120, 120˜140, 140˜160, 160˜180, 180˜200, 200˜220, 220˜240, 240˜260, 260˜280, 280˜300, 300˜320, 320˜340, 340˜360, 360˜380, 380˜400, 400˜420, 420˜440, 440˜460, 460˜480, or 480˜500.

p₃ can be in a range of 0.5˜50, 0˜0.5, 0.5˜1, 1˜2, 2˜4, 4˜6, 6˜8, 8˜10, 10˜12, 12˜14, 14˜16, 16˜18, 18˜20, 20˜25, 25˜30, 30˜35, 35˜40, 40˜45, or 45˜50.

q₃ can be in a range of 0˜500, 0˜1, 1˜2, 2˜4, 4˜6, 6˜8, 8˜10, 10˜12, 12˜14, 14˜16, 16˜18, 18˜20, 20˜25, 25˜30, 30˜35, 35˜40, 40˜45, 45˜50, 45˜50, 50˜60, 60˜70, 70˜80, 80˜90, 90˜100, 100˜120, 120˜140, 140˜160, 160˜180, 180˜200, 200˜220, 220˜240, 240˜260, 260˜280, 280˜300, 300˜320, 320˜340, 340˜360, 360˜380, 380˜400, 400˜420, 420˜440, 440˜460, 460˜480, or 480˜500.

r₃ can be in a range of 0˜200, 0˜1, 1˜2, 2˜3, 3˜4, 4˜5, 6˜7, 6˜7, 7˜8, 8˜9, 9˜10, 10˜15, 15˜20, 25˜30, 30˜35, 35˜40, 40˜45, 45˜50, 45˜50, 50˜60, 60˜70, 70˜80, 80˜90, 90˜100, 100˜120, 120˜140, 140˜160, 160˜180, 180˜200, or 200˜220.

The total number of all hydrophilic and hydrophobic groups can be described as r₃, where r_{3,A} is the total number of hydrophilic groups, and r_{3,B} is the total number of hydrophobic groups. Accordingly, (r_{3,A}+r_{3,B}) may be in a range of 1˜200, 1˜2, 2˜3, 3˜4, 4˜5, 6˜7, 6˜7, 7˜8, 8˜9, 9˜10, 10˜12, 12˜14, 14˜16, 16˜18, 18˜20, 20˜25, 25˜30, 30˜35, 35˜40, 40˜45, 45˜50, 50˜60, 60˜70, 70˜80, 80˜90, 90˜100, 100˜120, 120˜140, 140˜160, 160˜180, or 180˜200; r_{3,A} may be less than 151, or may be in a range of 1˜2, 2˜3, 3˜4, 4˜5, 6˜7, 6˜7, 7˜8, 8˜9, 9˜10, 10˜12, 12˜14, 14˜16, 16˜18, 18˜20, 20˜25, 25˜30, 30˜35, 35˜40, 40˜45, 45˜50, 50˜60, 60˜70, 70˜80, 80˜90, 90˜100, 100˜120, 120˜140, or 140˜150.

s₃₁ can be in a range of 1˜10, 1˜2, 2˜3, 3˜4, 4˜5, 6˜7, 6˜7, 7˜8, 8˜9, or 9˜10.

s₃₂ can be in a range of 1˜10, 1˜2, 2˜3, 3˜4, 4˜5, 6˜7, 6˜7, 7˜8, 8˜9, or 9˜10.

s₃₃ can be in a range of 1˜10, 1˜2, 2˜3, 3˜4, 4˜5, 6˜7, 6˜7, 7˜8, 8˜9, or 9˜10.

s₃₄ can be in a range of 1˜10, 1˜2, 2˜3, 3˜4, 4˜5, 6˜7, 6˜7, 7˜8, 8˜9, or 9˜10.

t₃₁ can be in a range of 1˜10, 1˜2, 2˜3, 3˜4, 4˜5, 6˜7, 6˜7, 7˜8, 8˜9, or 9˜10.

t₃₂ can be in a range of 1˜10, 1˜2, 2˜3, 3˜4, 4˜5, 6˜7, 6˜7, 7˜8, 8˜9, or 9˜10.

t₃₃ can be in a range of 1˜10, 1˜2, 2˜3, 3˜4, 4˜5, 6˜7, 6˜7, 7˜8, 8˜9, or 9˜10.

t₃₄ can be in a range of 1˜10, 1˜2, 2˜3, 3˜4, 4˜5, 6˜7, 6˜7, 7˜8, 8˜9, or 9˜10.

In a specific embodiment, in Formula III, m₃=22˜1136, n₃=10˜500, p₃=1˜50, q₃=0, r₃=0. For products prepared by these polymers (for example, polymer particles), the fluorescent molecules distributed in the hydrophobic core do not emit light under certain excitation conditions (e.g., with NIR as the excitation light source) due to the FRET (Fluorescence Resonance Energy Transfer) effect. However, after being administered to an individual, the polymer particles can be enriched at the target site (for example, tumor site) through EPR (Enhanced Permeation and Retention) passive targeting (or other tissue uptake methods). Because the target site has a special pH environment (for example, acidic environment), the protonatable group (i.e., the A3 group) can be protonated in this pH range, and the charge repulsion generated by its protonation and the increase in polymer solubility drive the disintegration of the polymer particles. The FRET effect of the fluorescent group on each macromolecular segment is reduced or even completely eliminated. The polymer molecules in the dispersed state enriched at the target site can emit fluorescence under certain excitation conditions (for example, with the near-infrared ray as the excitation light source).

In a preferred embodiment, the chemical structural formula of the functionalized diblock copolymer is shown as one of the following:

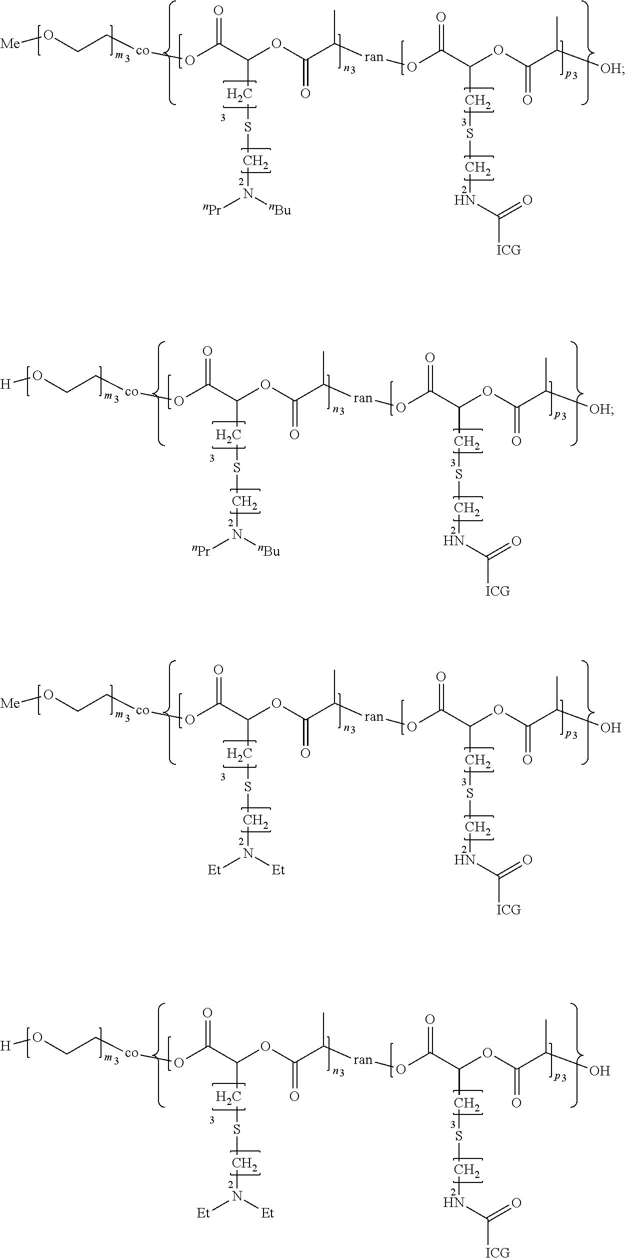

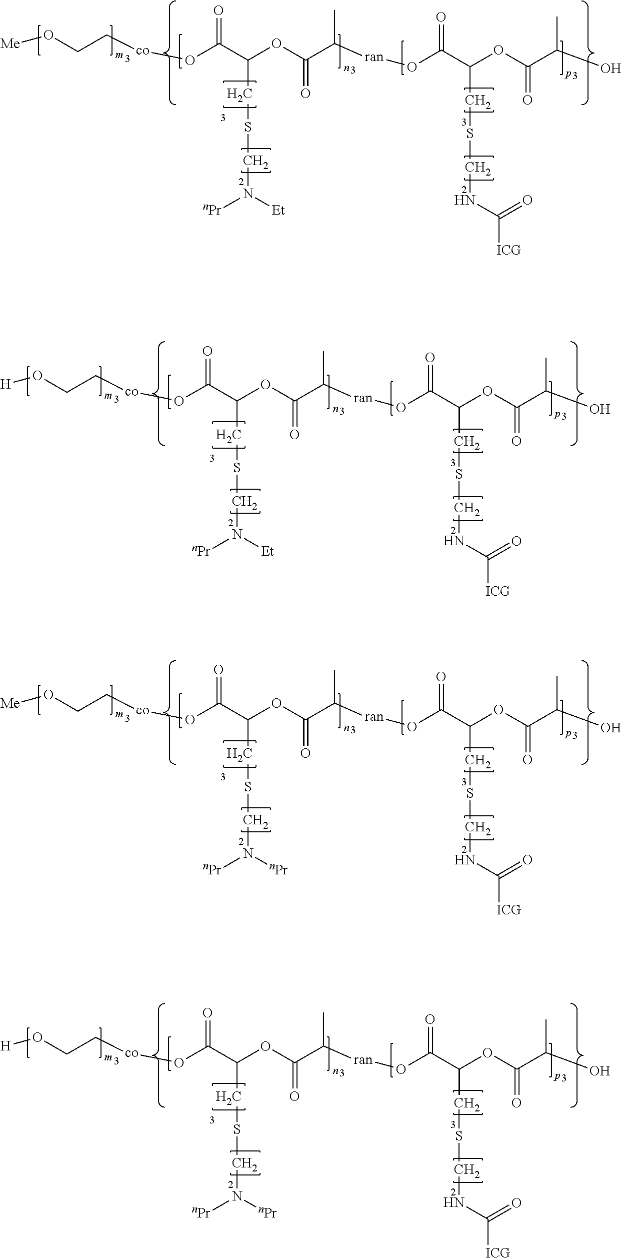

wherein m₃ is in a range of 22˜1136, preferably 44˜226, n₃ is in a range of 10˜500, preferably 30˜200, and p₃ is in a range of 0.5˜5.

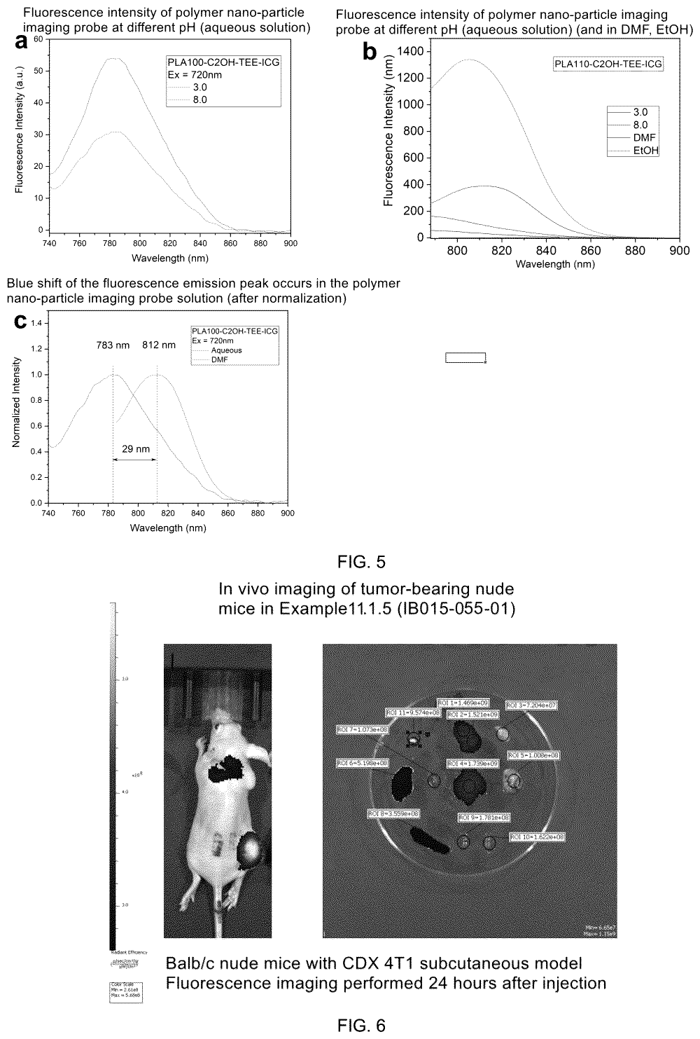

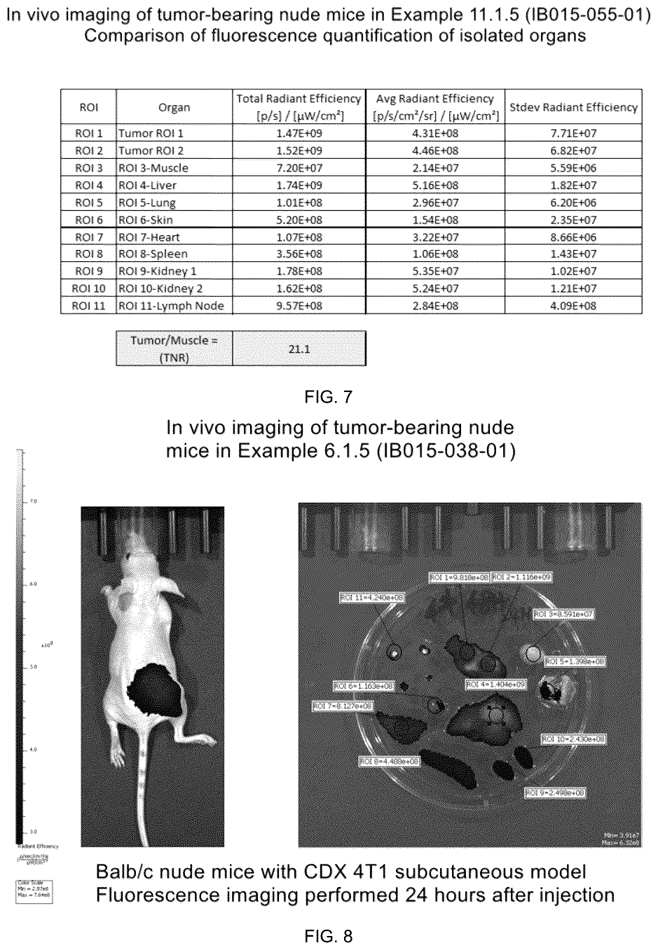

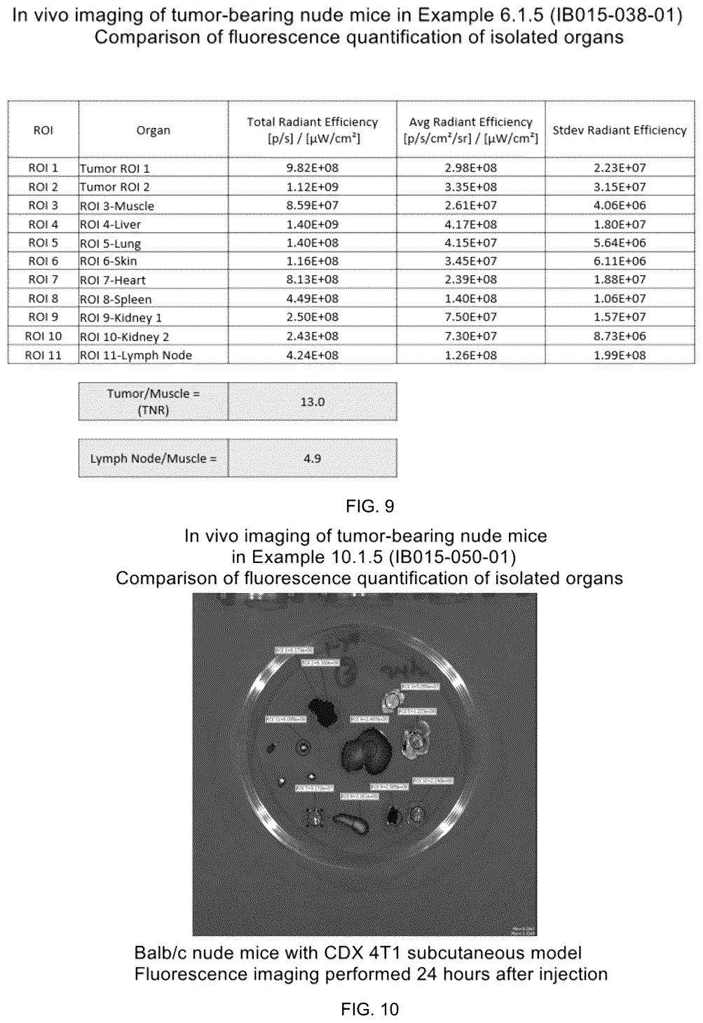

In a specific embodiment of the present disclosure, in Formula III, m₃=22˜1136, n₃=10˜500, p₃=0.5˜50, q₃=0, r₃=1˜200. For the products prepared by these polymers (for example, polymer particles), the fluorescent molecules distributed in the hydrophobic core do not emit light under certain excitation conditions (for example, in the case of near-infrared ray as the excitation light source) due to the FRET effect. The addition of hydrophilic/hydrophobic groups (i.e., E₃ groups) increases the stability of polymer particles, enhances the FRET effect of polymer particles (more complete fluorescence quenching), and changes the acidity sensitivity of polymer particles. However, after being administered to an individual, the polymer particles can be enriched at the target site (for example, tumor site) through EPR passive targeting (or other tissue uptake methods). Because the target site has a special pH environment (for example, acidic environment), the protonatable group (i.e., the A₃ group) can be protonated in this pH range, and the charge repulsion generated by its protonation and the increase in polymer solubility drive the disintegration of the polymer particles. The FRET effect of the fluorescent group on each macromolecular segment is reduced or even completely eliminated. The polymer molecules in the dispersed state enriched at the target site can emit fluorescence under certain excitation conditions (for example, with the near-infrared ray as the excitation light source). The experimental results showed that compared to the polymer without hydrophobic group E3, Example 6.1.5 introduced twenty —C₉H₁₉ groups through the side chain on the hydrophobic block of the polymer, which resulted in a higher degree of quenching of ICG in the self-organized state of the aqueous solution. Changing the solution pH to more acidic conditions still did not fully disintegrate the polymer (as evidenced by the relatively low fluorescence intensity of the acidic solution and the large increase in fluorescence intensity that occurred when DMF was added to the polymer solution). The polymer (IB015-038-01) of Example 6.1.5 has achieved highly specific tumor fluorescence labeling (TNR=13) for tumor-bearing mice in in vivo imaging experiments, as shown in FIGS. 8 and 9. Additional experimental results showed that after the introduction of the hydrophilic side chain —C₂H₄—OH (Example 11.2.5), the emission of the polymer with the ICG attached underwent a large blue shift (as shown in the FIG. 5), presumably due to the fact that the hydroxyl group of the hydrophilic side chain improves the relative hydrophilicity of the hydrophobic block while also providing the ability to form a hydrogen-bonding interaction with the oxygen on the —C(′O)— of the ester bond on the polymer's main chain. This leads to a greater degree of tightening of the polymer in the core, and this tightened aggregation state causes a change in the molecular state of the ICG, which in turn leads to a change in its emission wavelength. Highly specific tumor fluorescent labeling (TNR=21) for tumor-bearing mice was also achieved in in vivo imaging experiments using Example 11.1.5 polymer (IB015-055-01, with twenty-four —C₂H₄—OH), as shown in the FIGS. 6 and 7.

In a preferred embodiment, the chemical structural formula of the functionalized diblock copolymer is shown as one of the following:

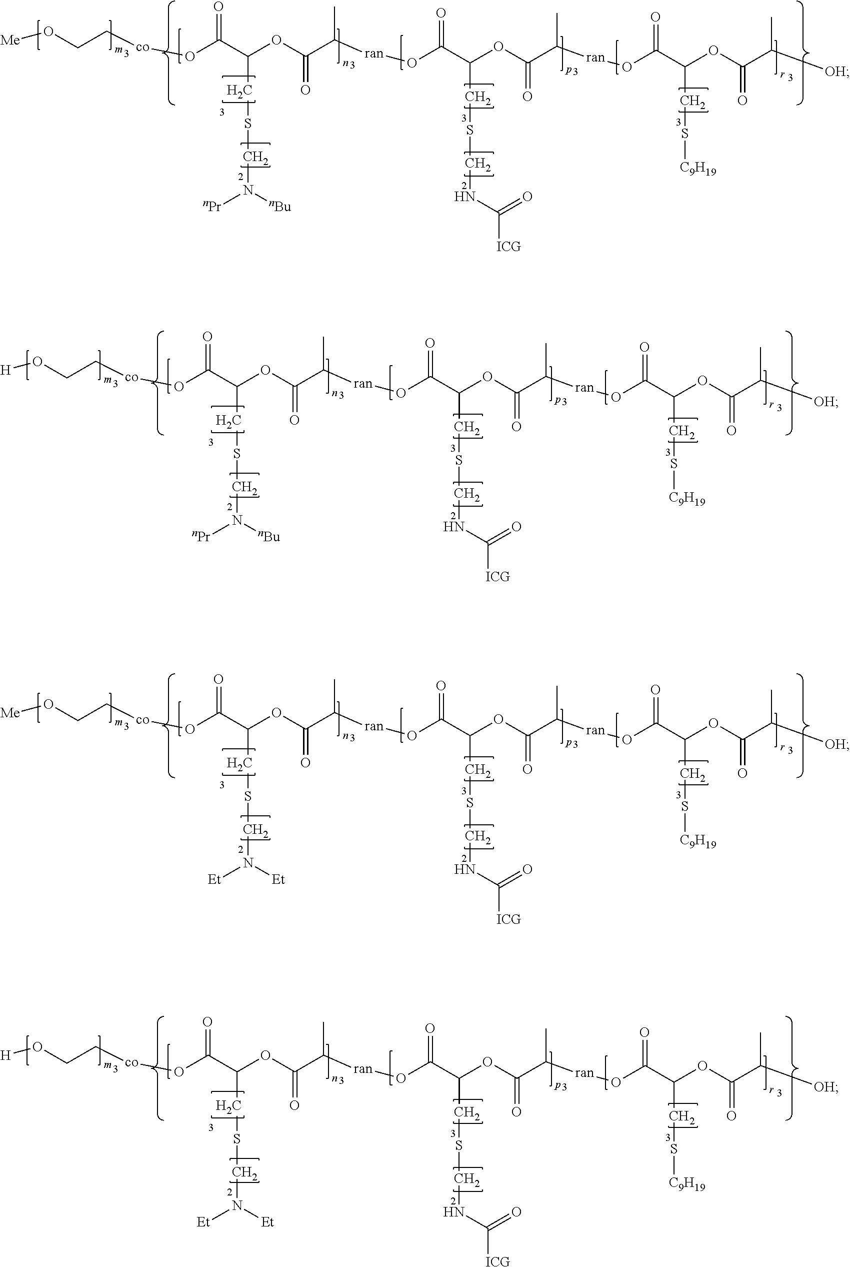

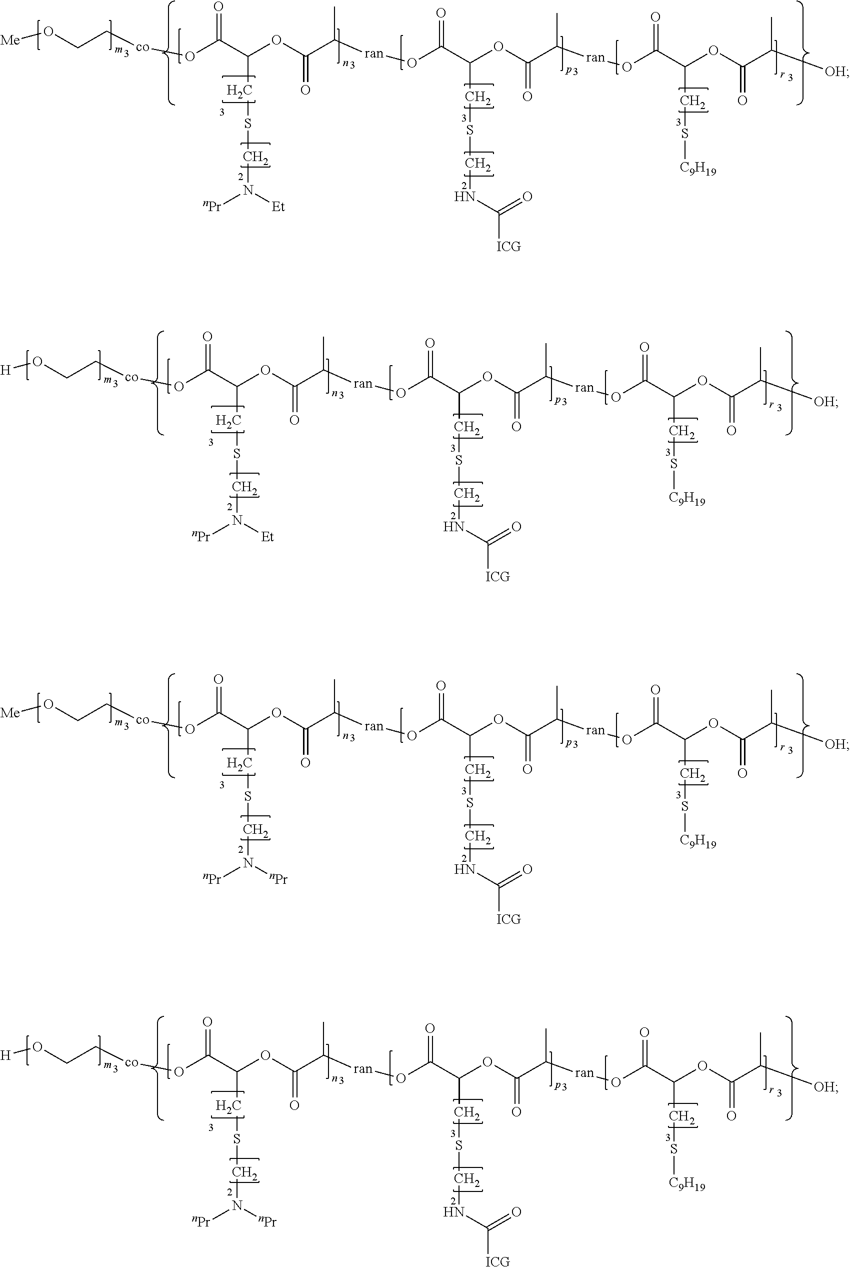

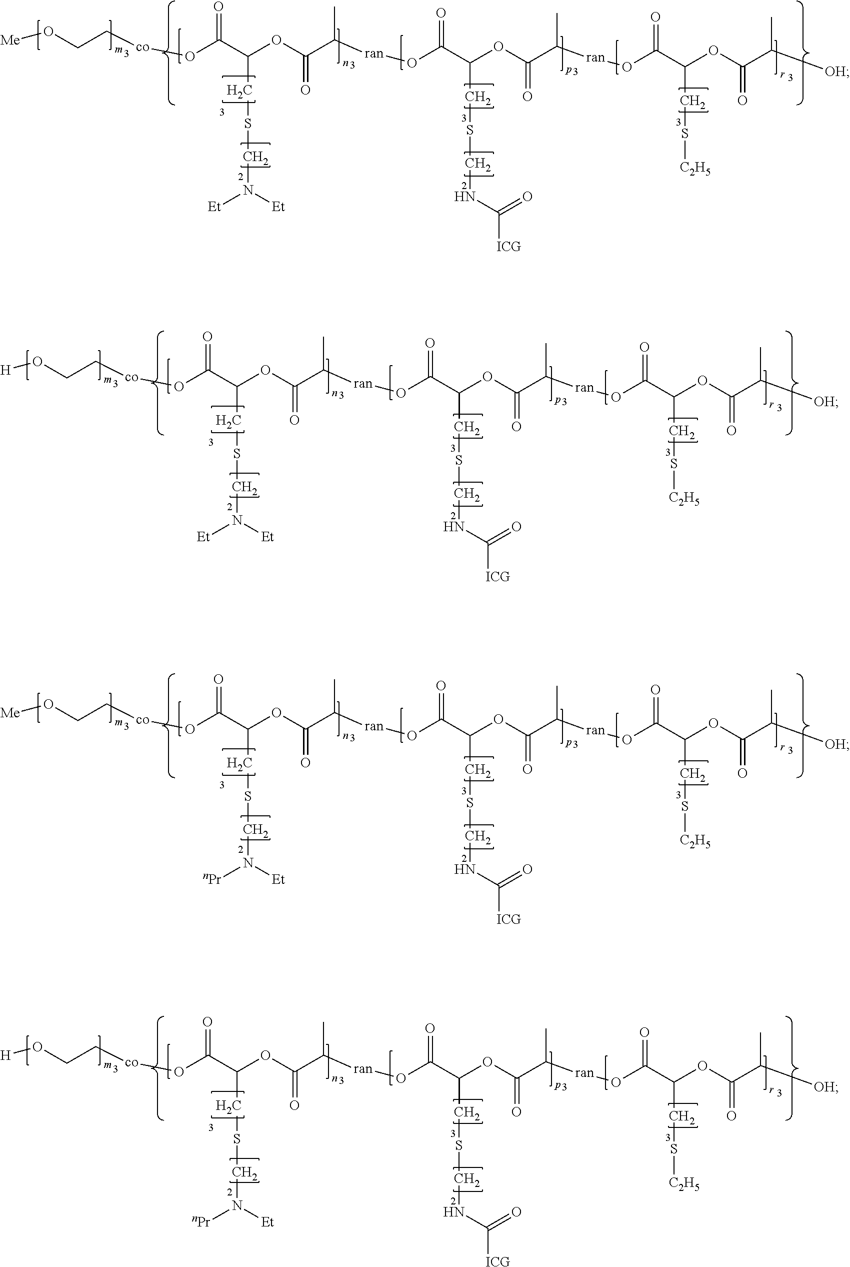

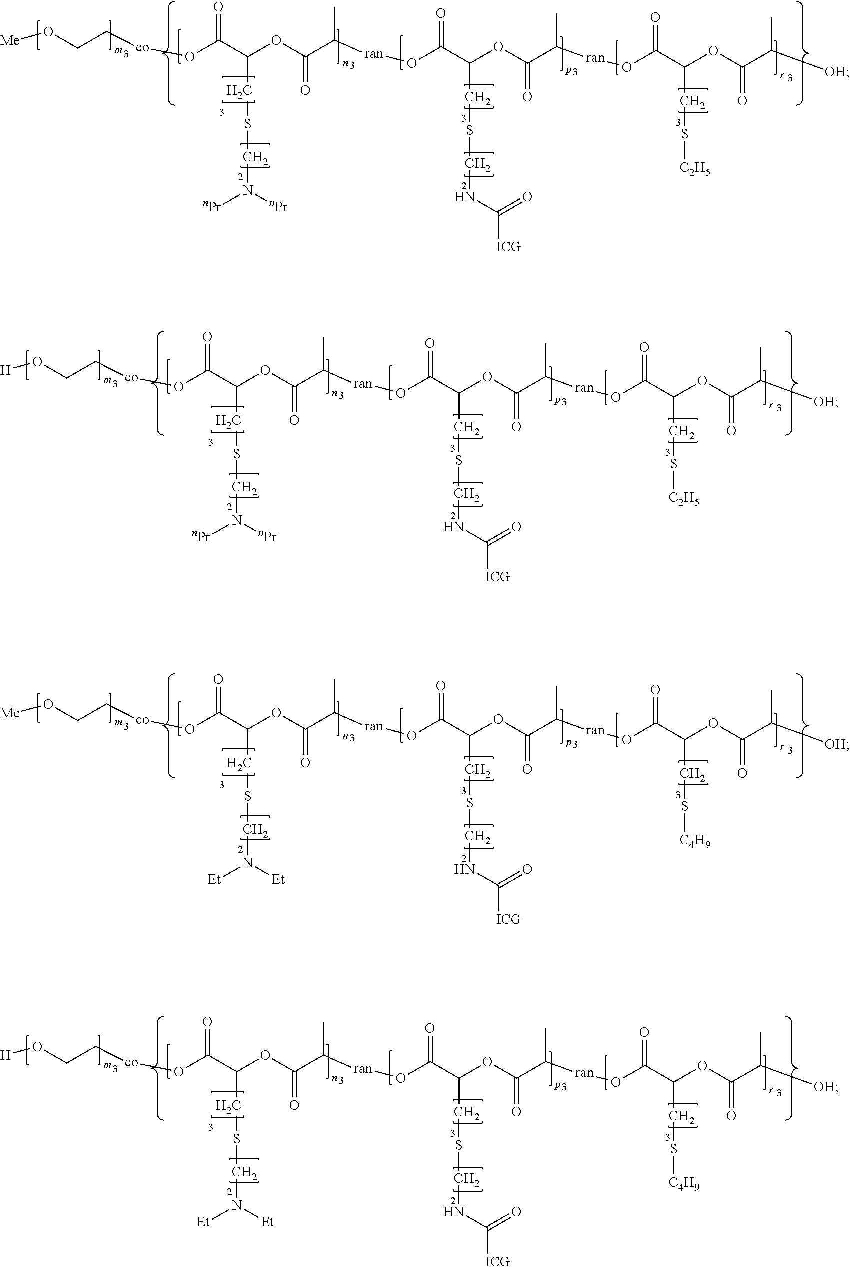

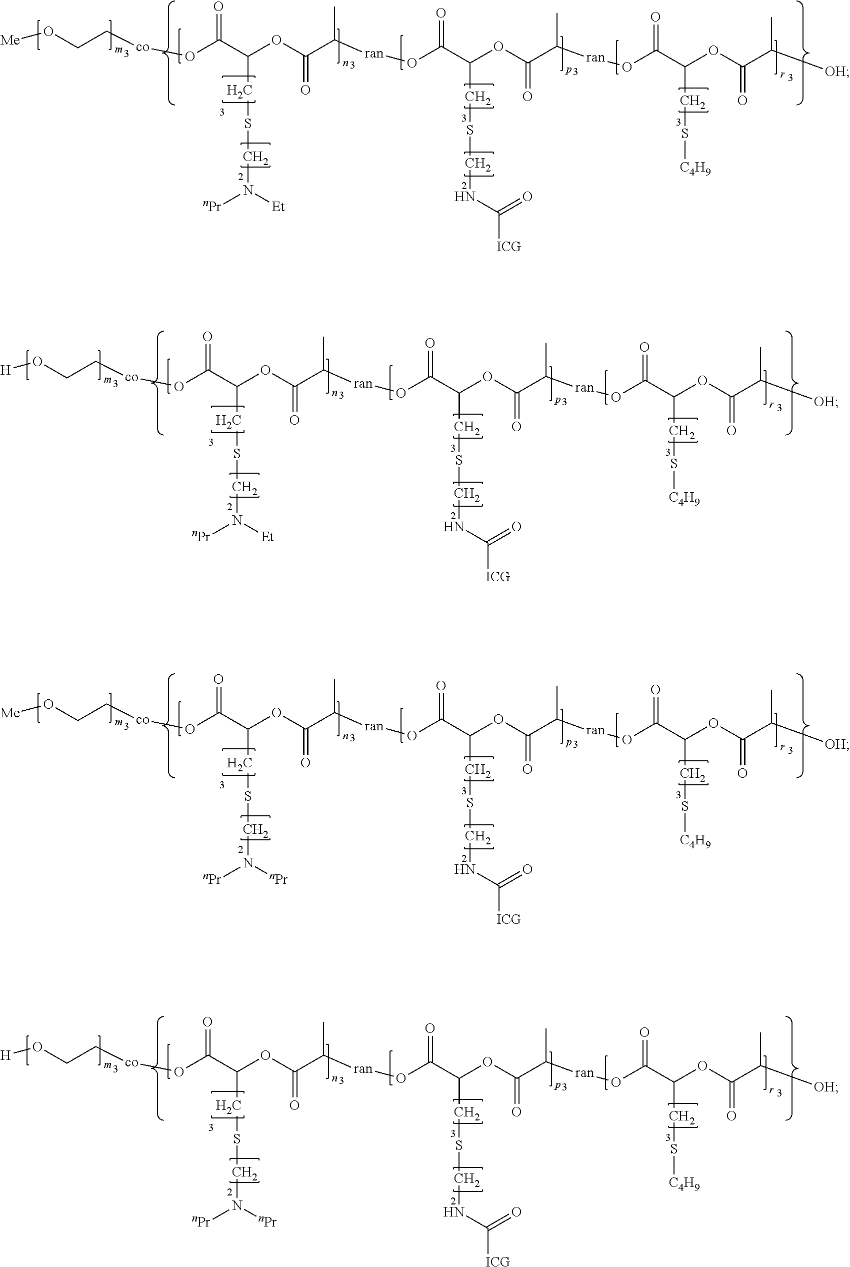

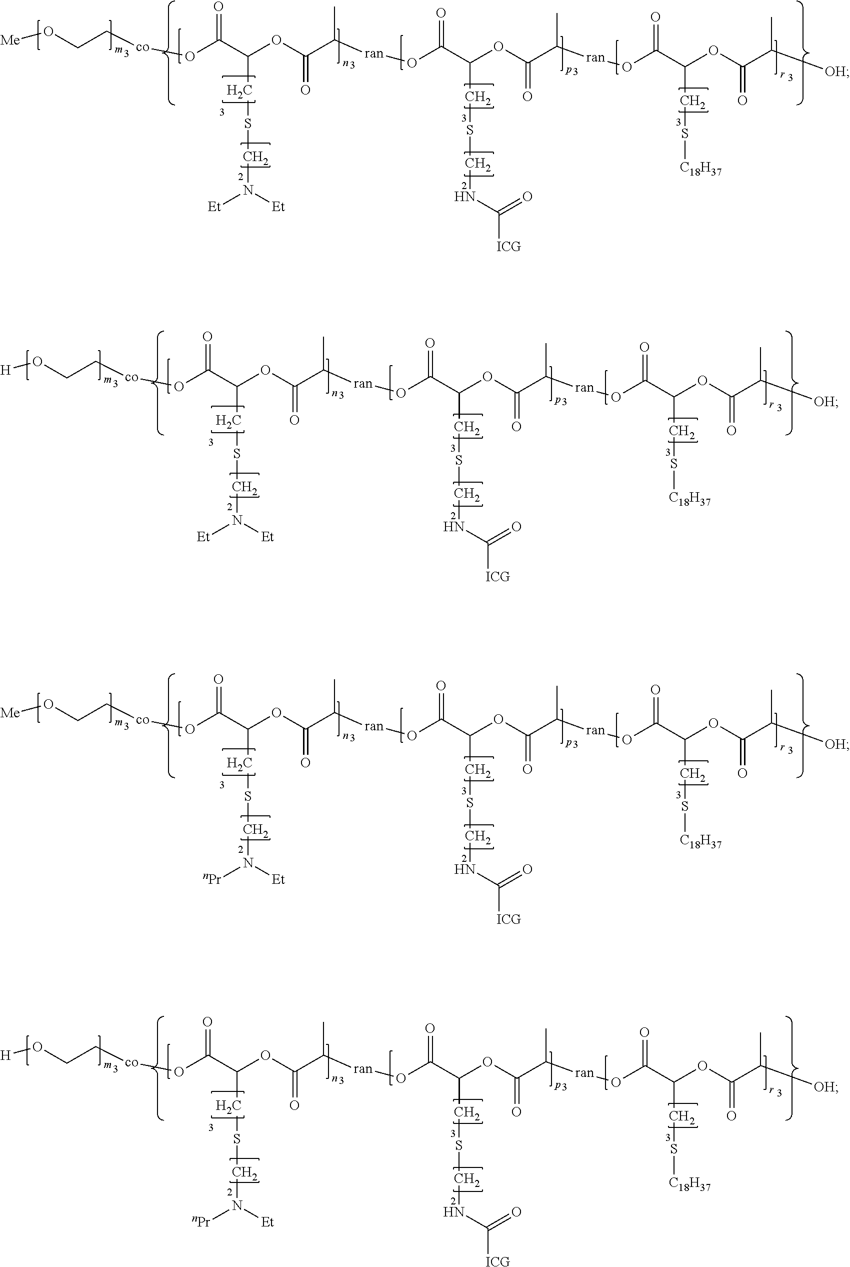

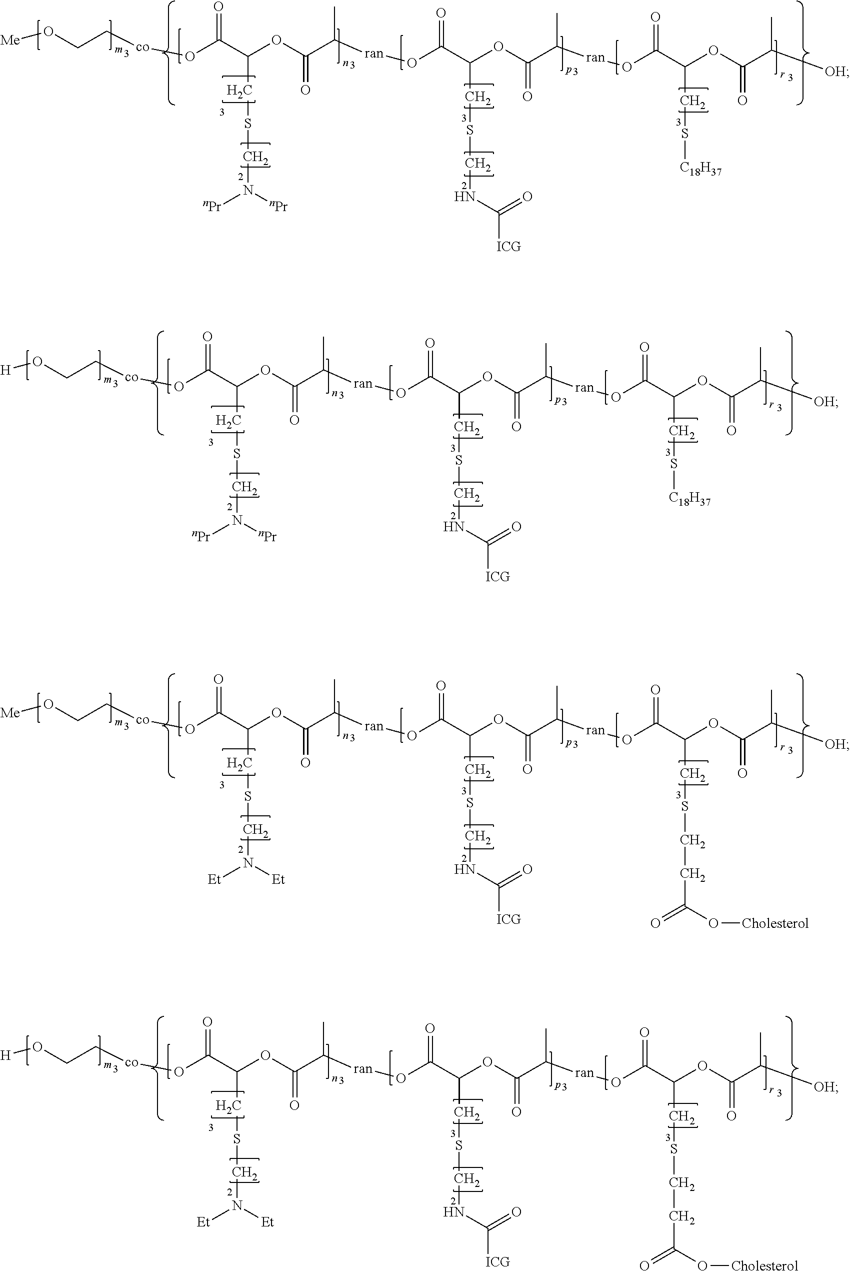

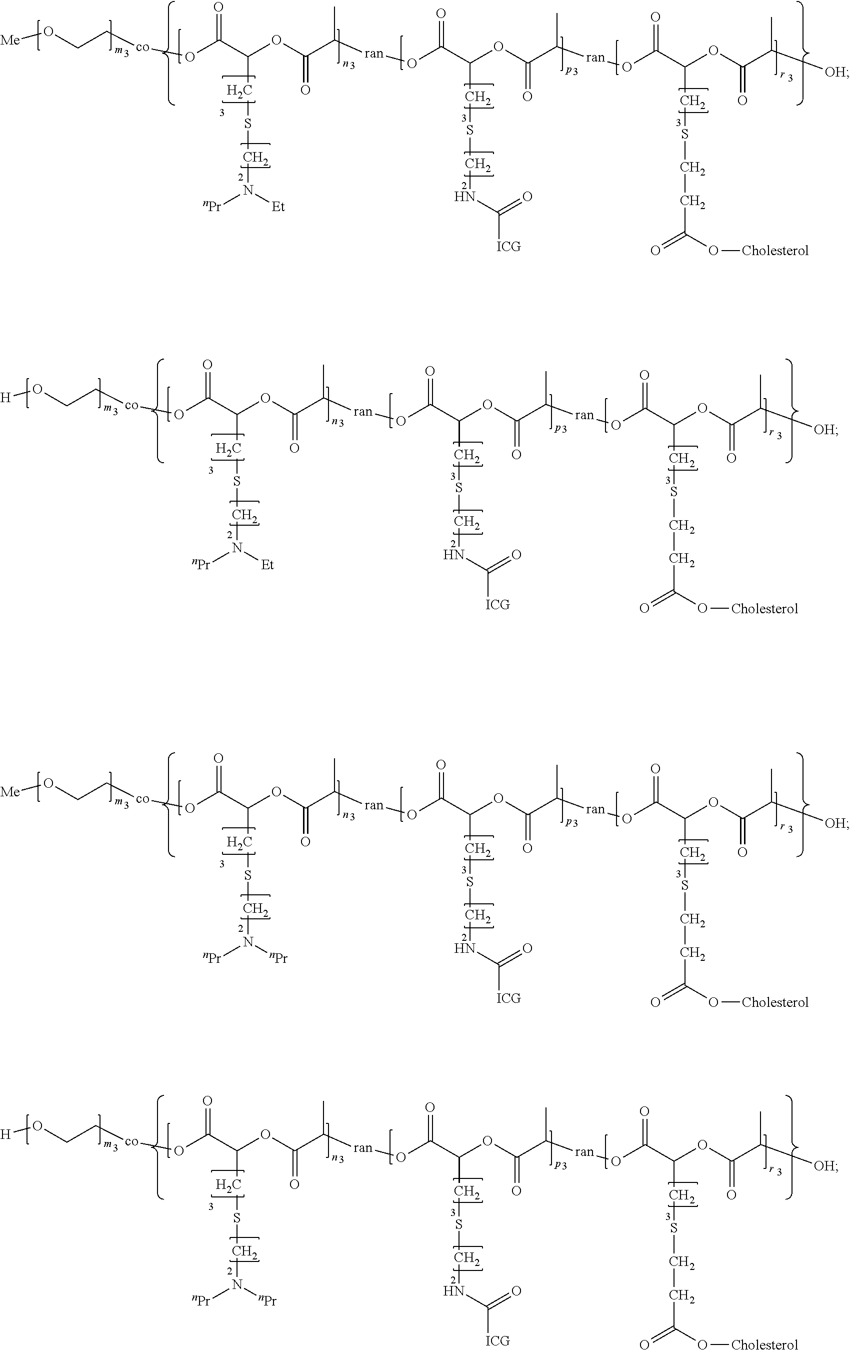

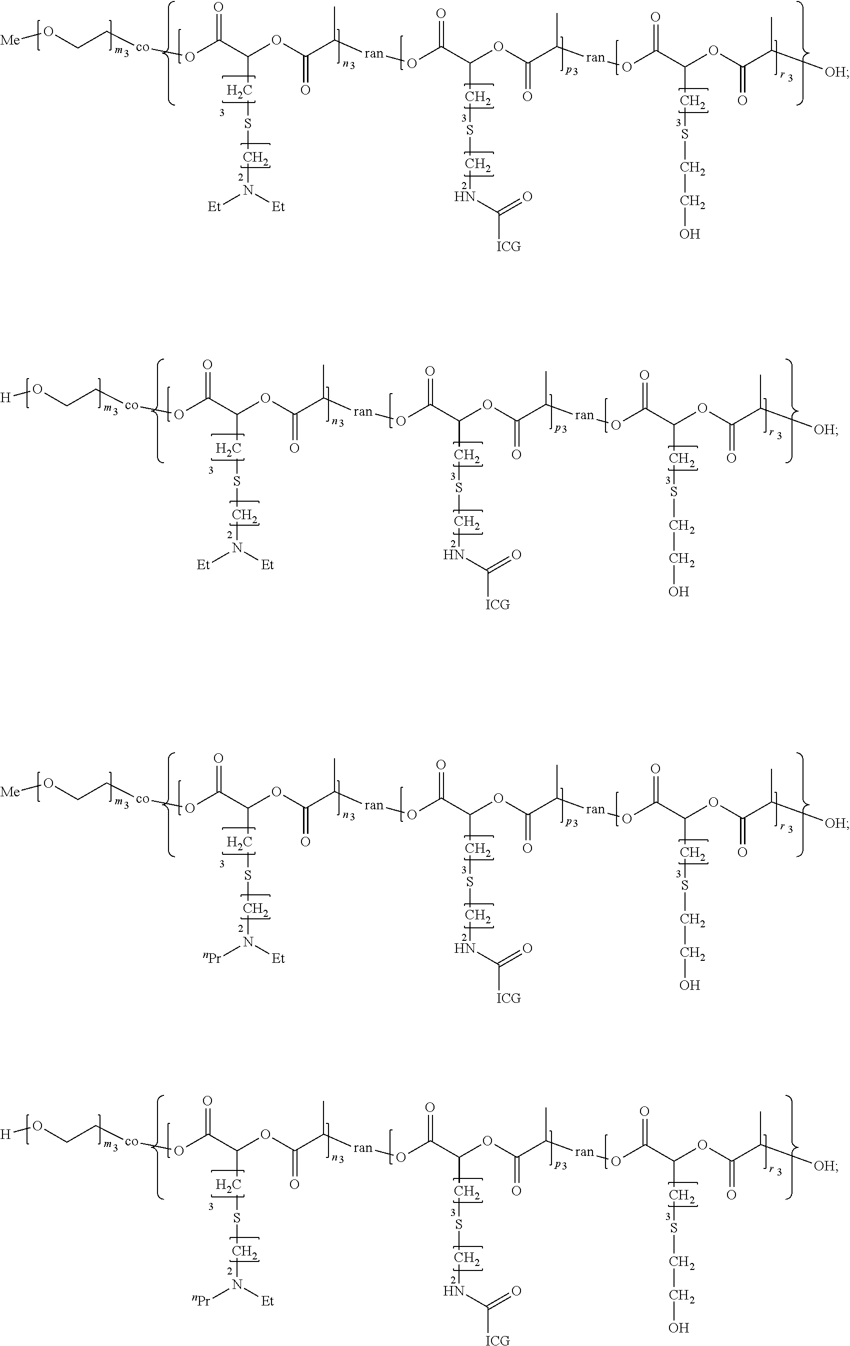

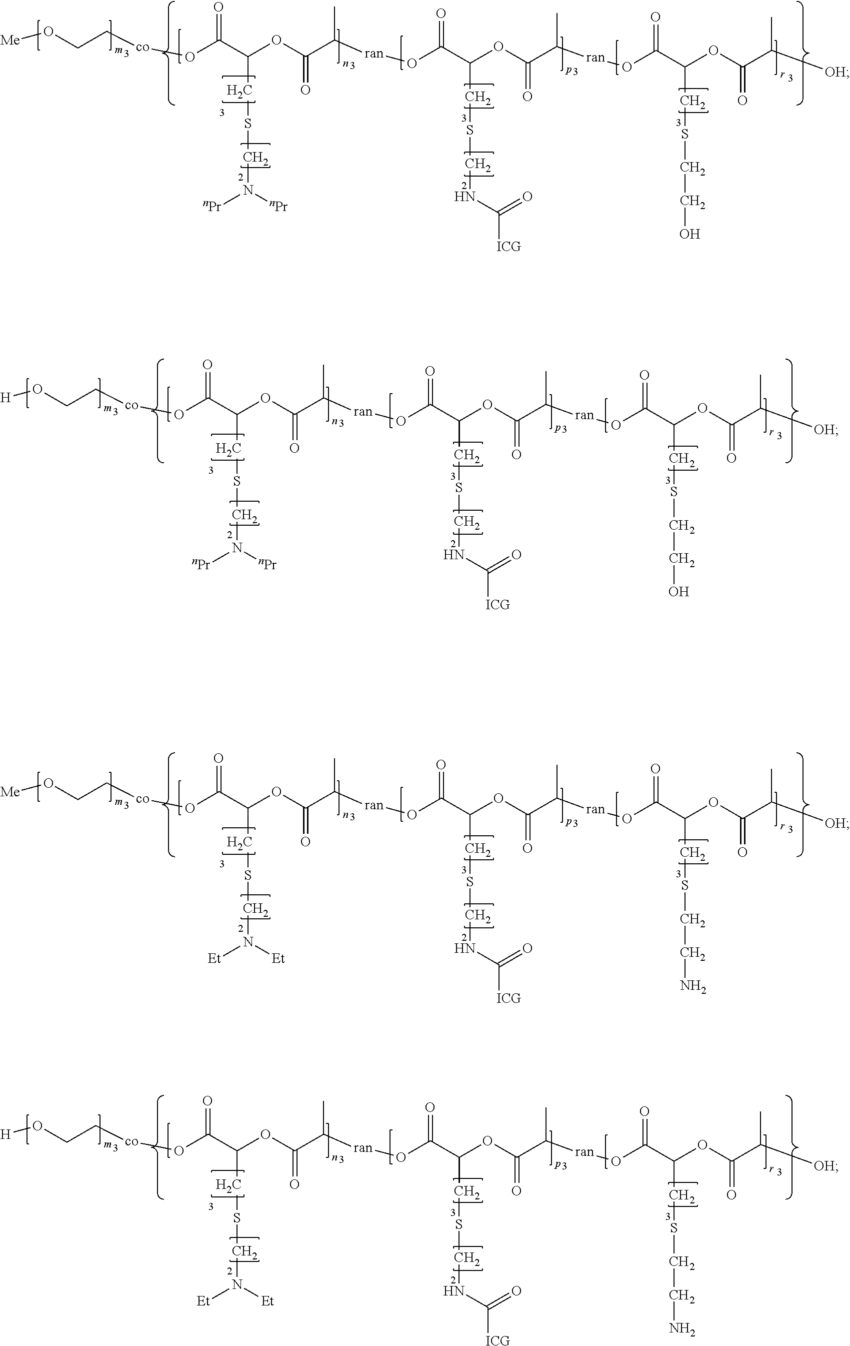

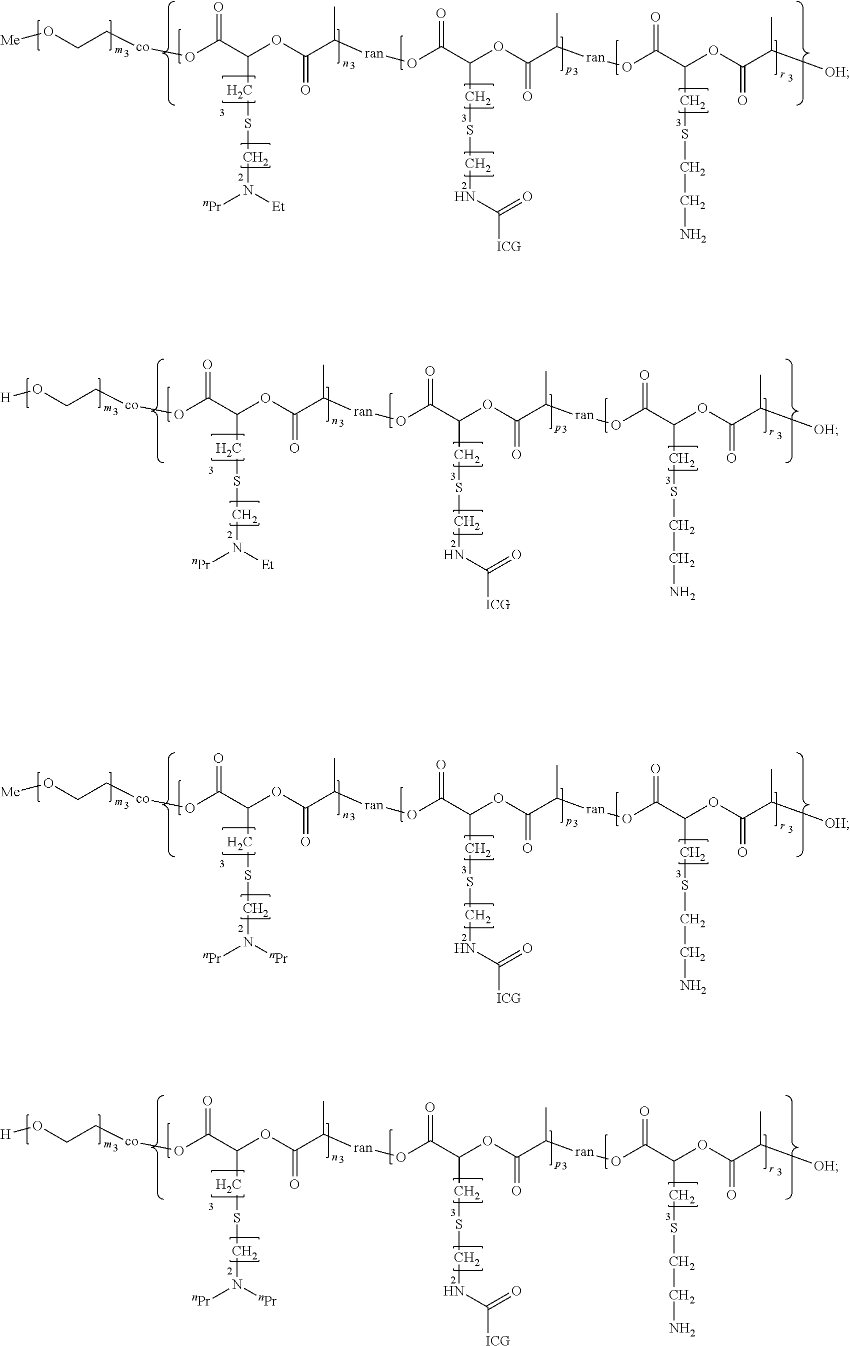

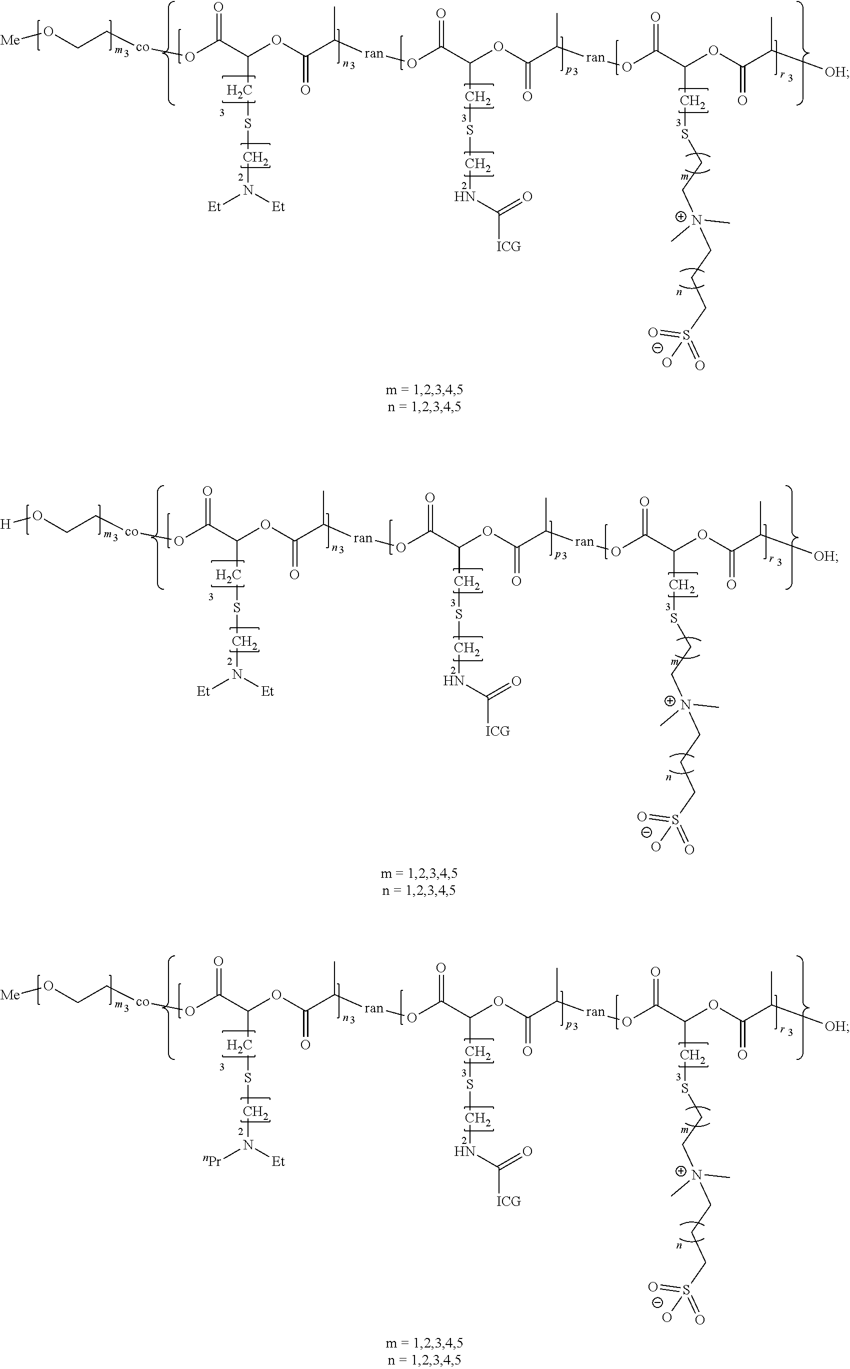

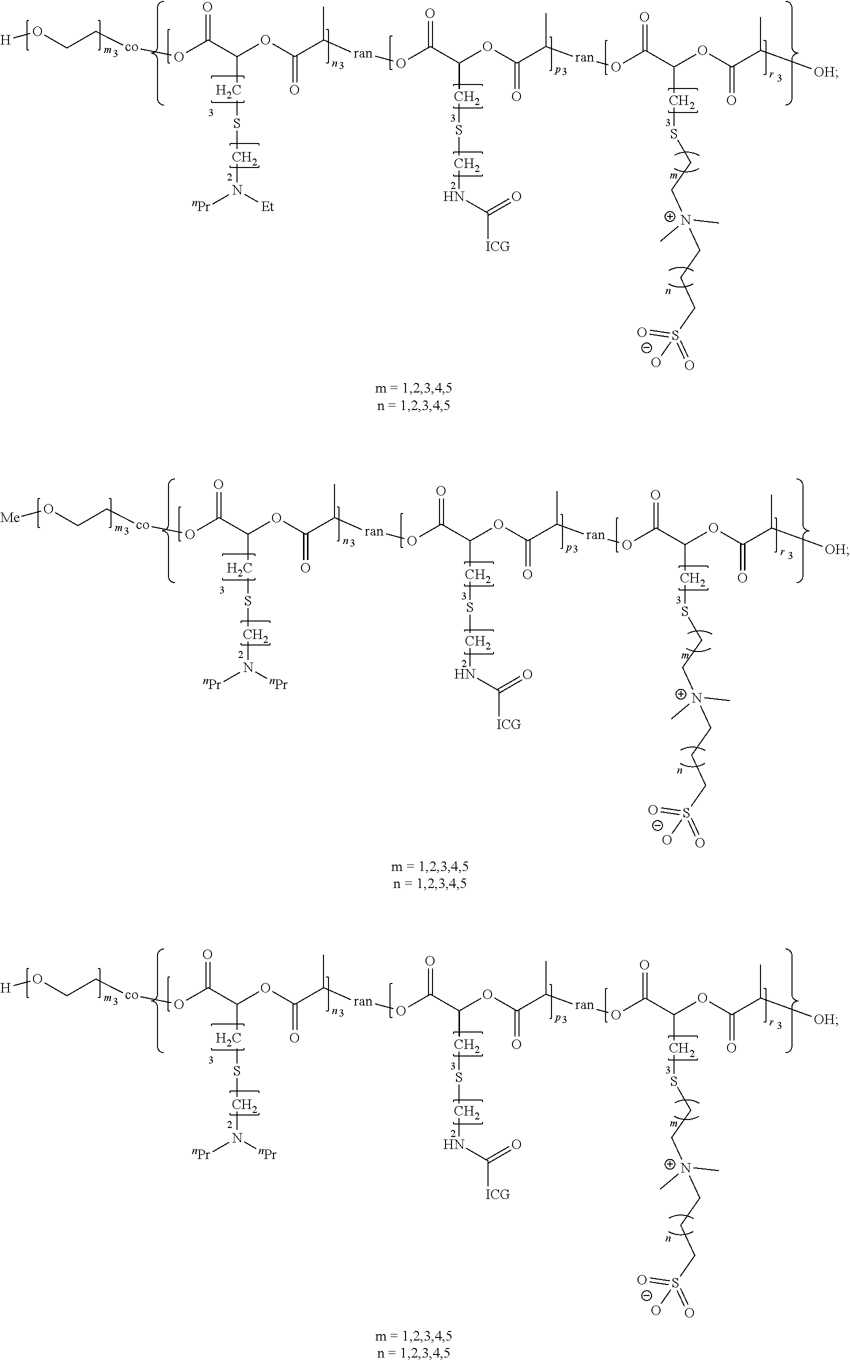

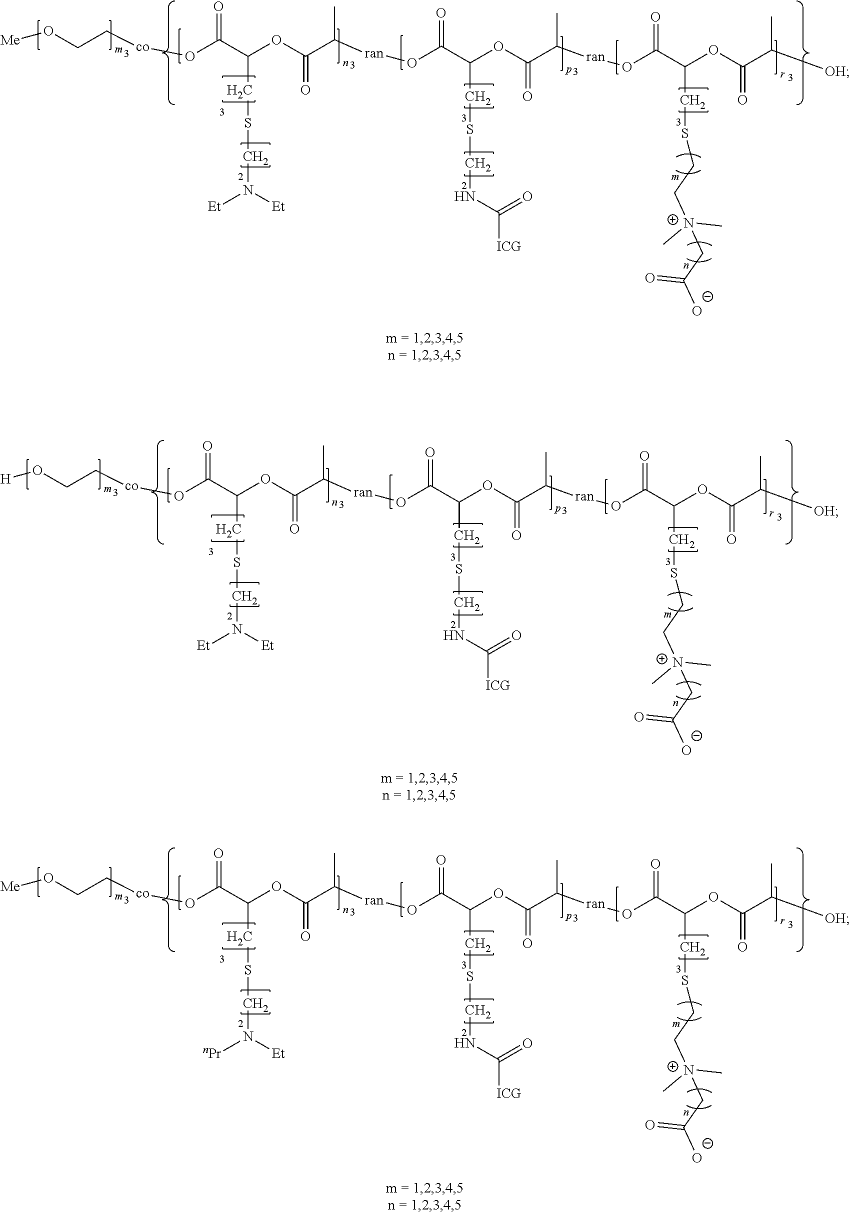

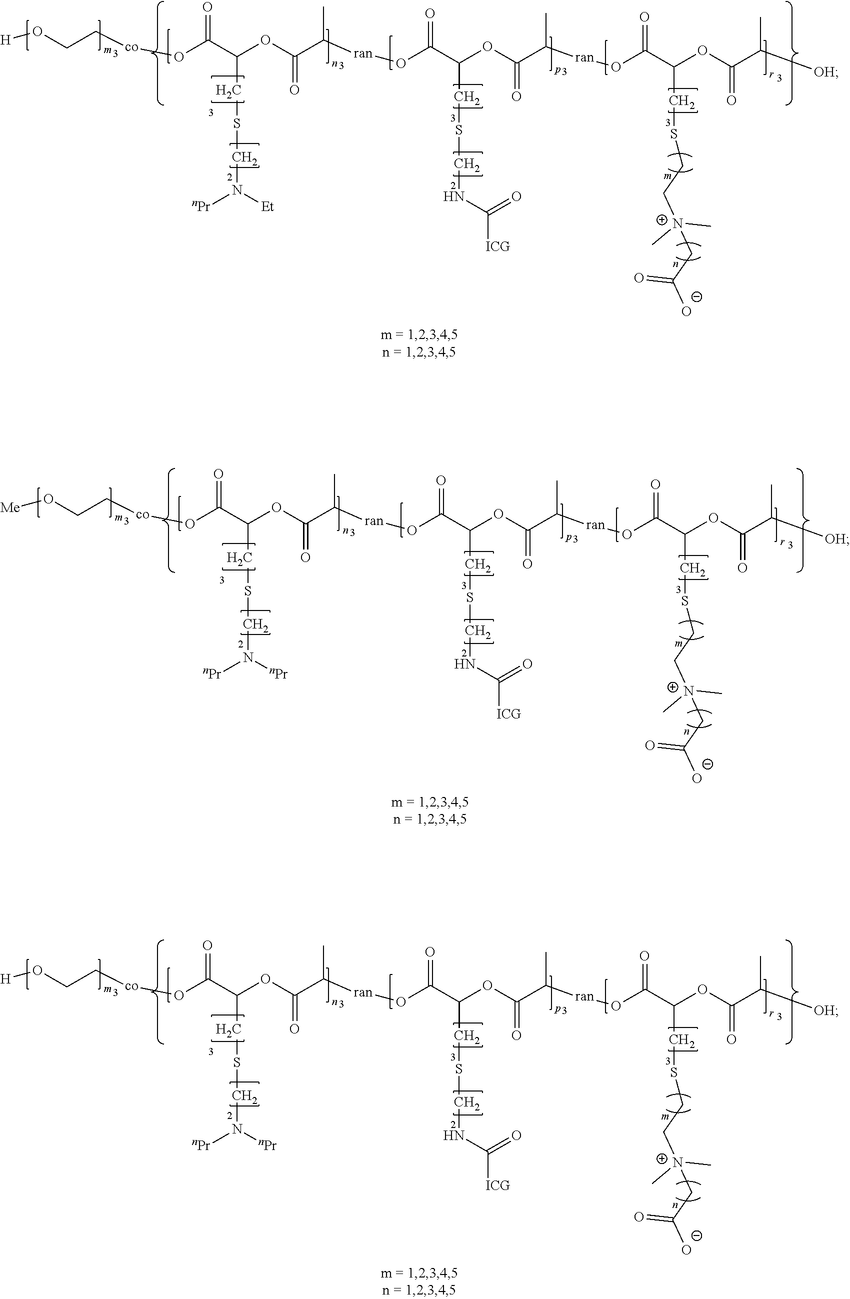

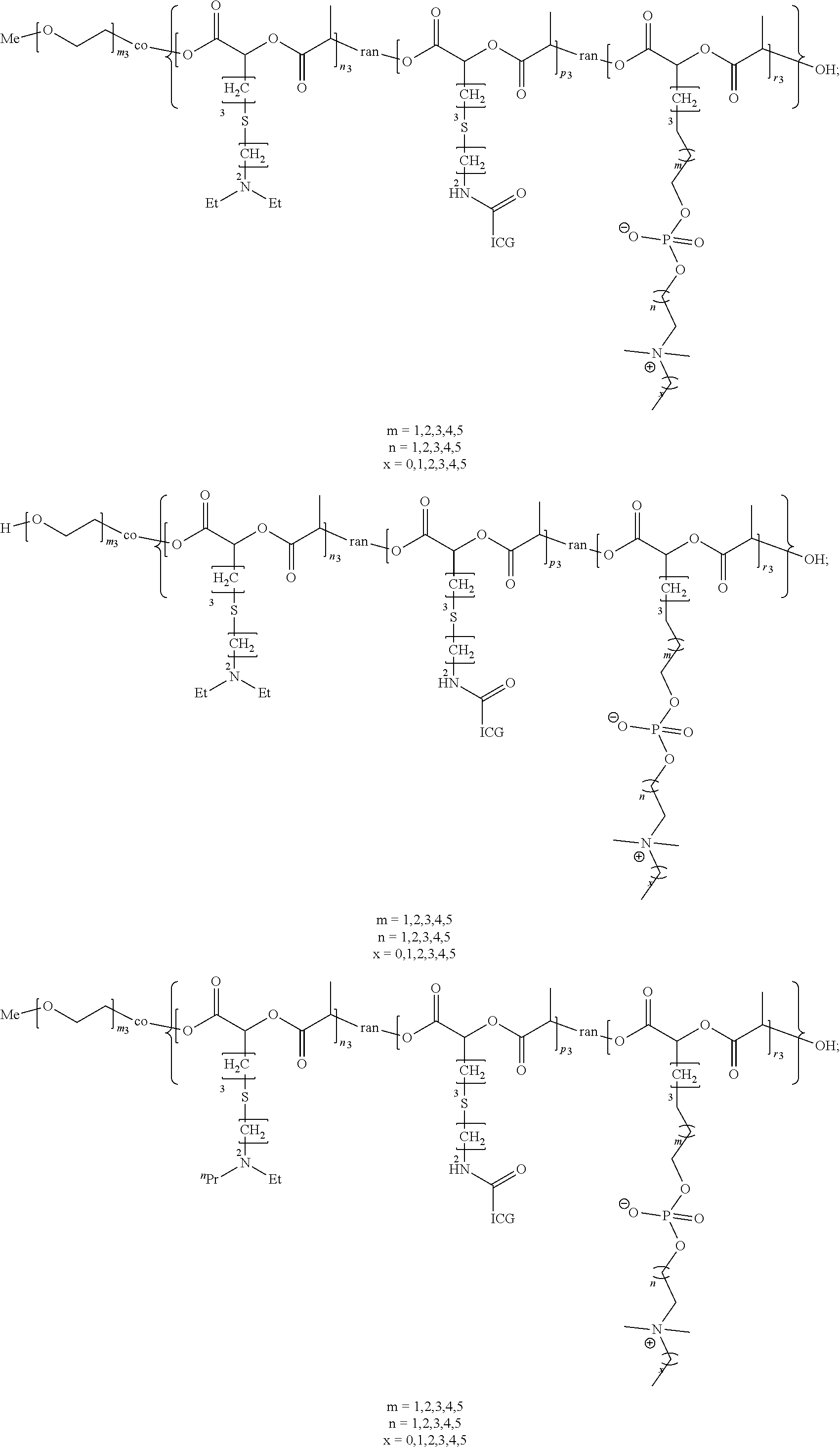

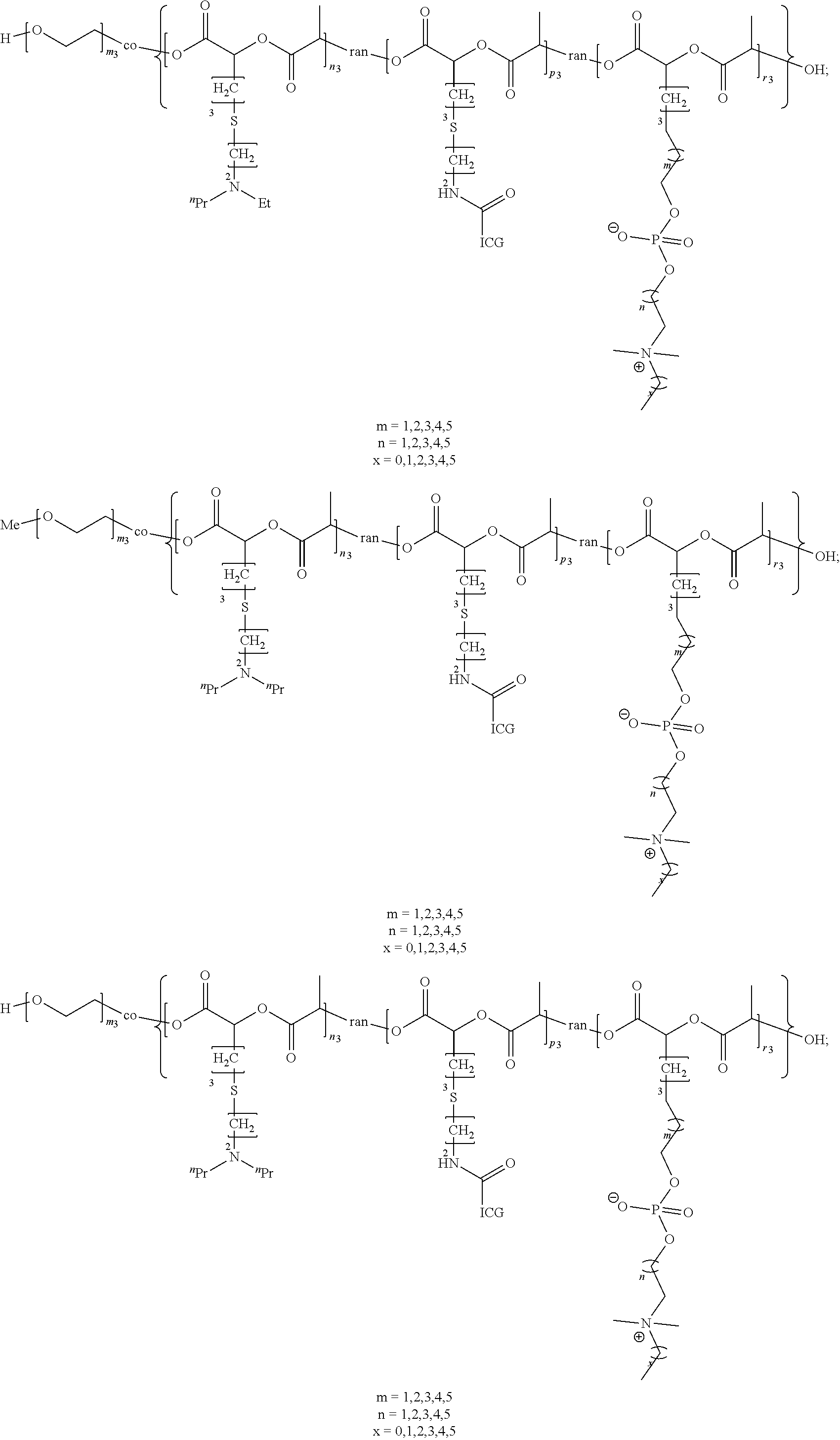

wherein m₃ is in a range of 22˜1136, preferably 44˜226, n₃ is in a range of 10˜500, preferably 30˜200, p₃ is in a range of 0.5˜5, and r₃ is in a range of 1˜200.

In a preferred embodiment, the chemical structural formula of the functionalized diblock copolymer is shown as one of the following:

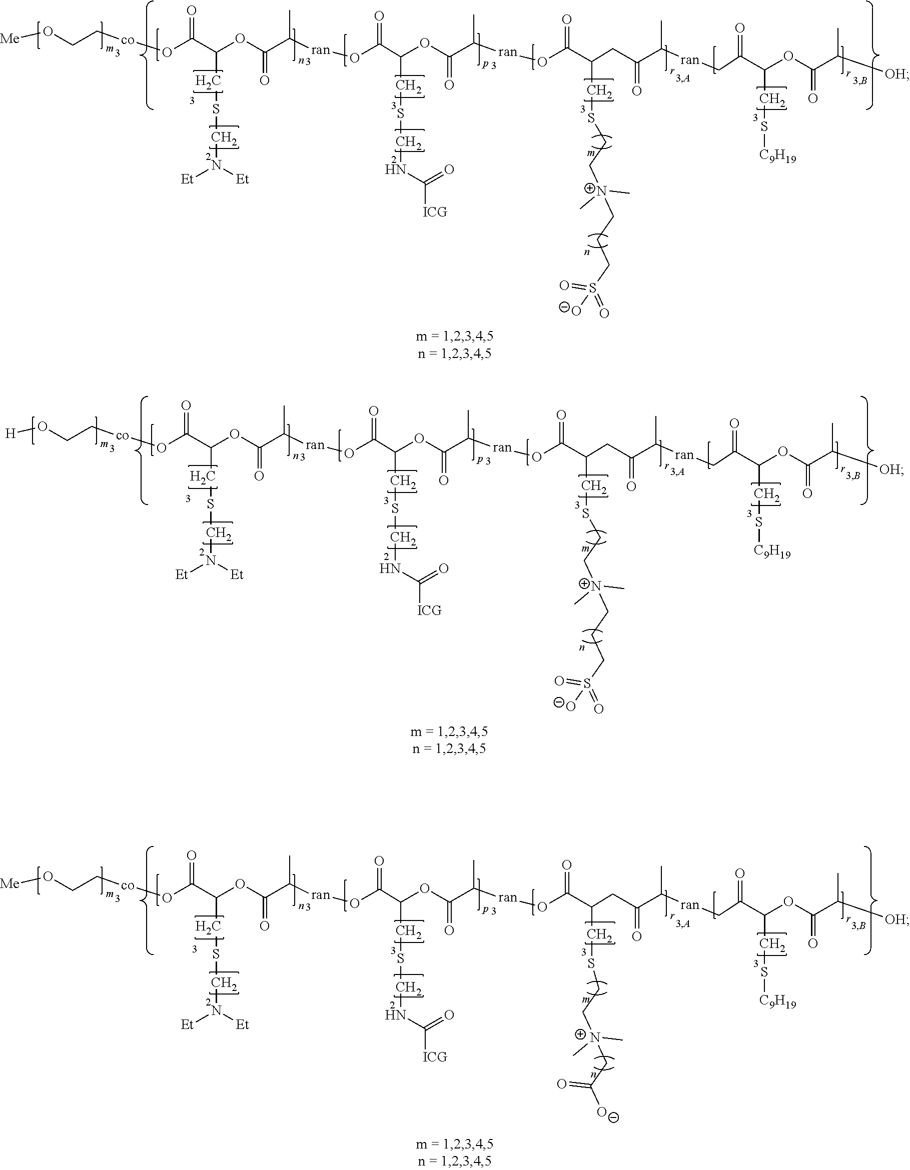

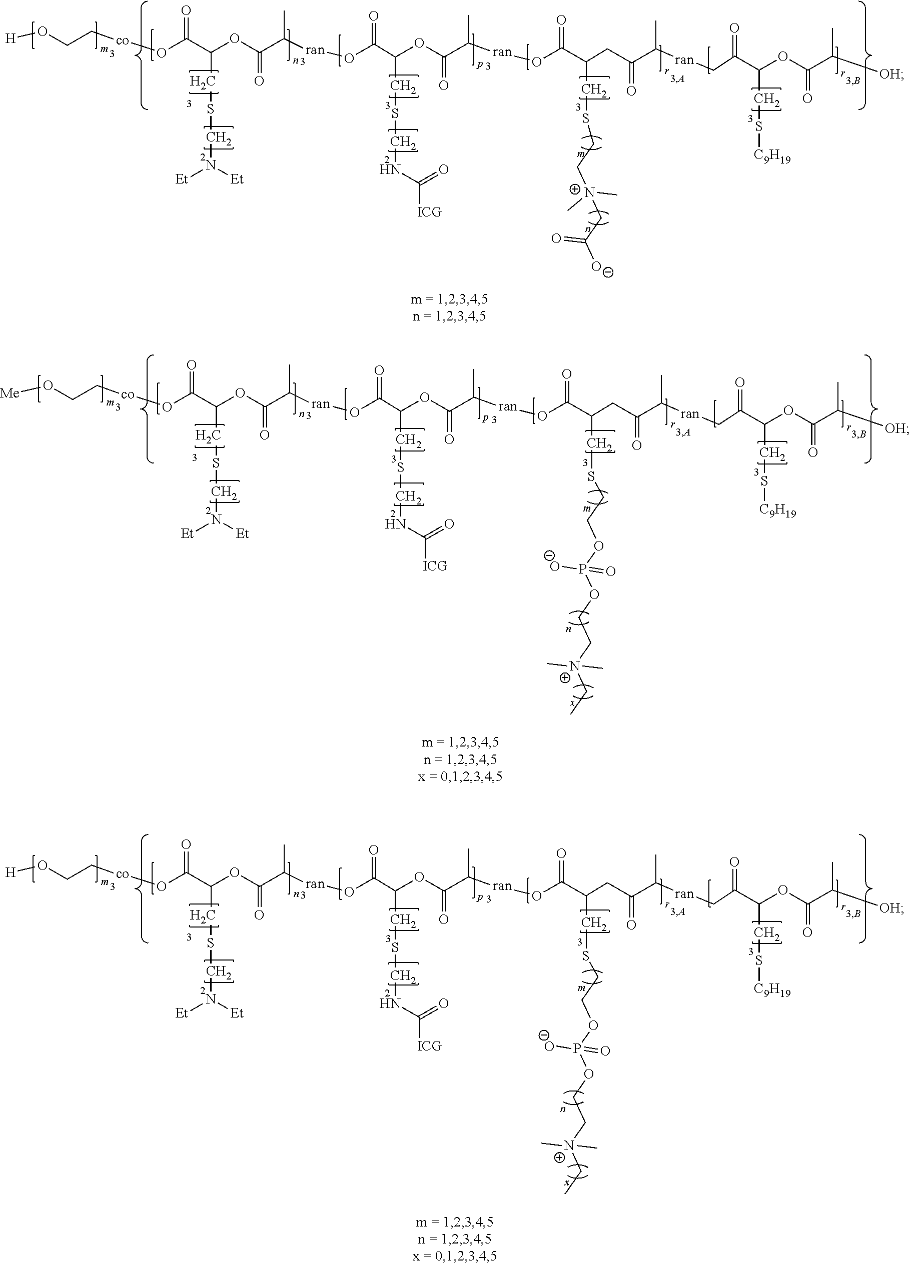

wherein m₃ is in a range of 22˜1136, preferably 44˜226, n₃ is in a range of 10˜500, preferably 30˜200, p₃ is in a range of 0.5˜5, (r_{3,A}+r_{3,B}) is in a range of 1˜200.

In another preferred embodiment, m₃=44˜226, n₃=50˜200, p₃=0.5˜5, and r₃=10˜40.

In a specific embodiment, in Formula III, m₃=22˜1136, n₃=10˜500, p₃=0.5˜50, q₃=1˜500, r₃=0. For the products prepared by these polymers (for example, polymer particles), the fluorescent molecules distributed in the hydrophobic core do not emit light under certain excitation conditions (for example, in the case of near-infrared ray as the excitation light source) due to the FRET effect. The delivery molecular group (i.e., the D₃ group) is connected to the main chain of the functionalized diblock copolymer. However, after being administered to an individual, the polymer particles can be enriched at the target site (for example, tumor site) through EPR passive targeting (or other tissue uptake methods). Because the target site has a special pH environment (for example, acidic environment), the protonatable group (i.e., the A₃ group) can be protonated in this pH range, and the charge repulsion generated by its protonation and the increase in polymer solubility drive the disintegration of the polymer particles. The FRET effect of the fluorescent group on each macromolecular segment is reduced or even completely eliminated. The polymer molecules in the dispersed state enriched at the target site can emit fluorescence under certain excitation conditions (for example, with the near-infrared ray as the excitation light source). In addition to the fluorescent molecular groups carried by the polymer particles, the delivery molecular groups attached to the side chains can continue to be hydrolyzed to the corresponding molecules under specific pH conditions at the target site after polymer disintegration. These molecules can play a corresponding role at the target site. For example, the delivery molecular group can be the group corresponding to 5-ALA, which can provide 5-ALA molecules after hydrolysis. 5-ALA can be efficiently enriched inside cancer cells with accelerated metabolism within a few hours and complete biosynthesis to form Protoporphyrin (Protoporphyrin remains/“traps” inside the cancer cells for a longer period of time after entering the cancer cells because its metabolic processes are blocked.). At this time, fluoresce can be efficiently emitted under the irradiation of near-infrared or 400 nm excitation light. Together with ICG fluorescent molecules (emitted at 780 nm), it is possible to realize the effect of separately exciting fluorescence at dual wavelengths, and to realize the fluorescence image enhancement of the tumor site, the confirmation of the tumor boundary, and the confirmation of cancerous or non-cancerous lesion. Moreover, 5-ALA is an approved precursor of photodynamic therapy drugs. In this embodiment, we creatively introduce and deliver 5-ALA, which not only enhances the effect of tumor-specific imaging, but also performs photodynamic therapy at tumor sites at the same time. In addition to the fluorescent molecular groups carried by the polymer particles, the insoluble anticancer drugs connected to the side chains can form a good water-soluble, safe and stable pharmaceutical injection preparation. On the one hand, this pharmaceutical preparation greatly increases the solubility of hydrophobic drugs in the blood and reduces their direct contact with the blood, which improves the stability of the drug in the body, reduces the toxic and side effects of the drug, and retains the high anti-tumor activity characteristics of the drug. After the polymer is disintegrated, the delivery molecular groups attached to the side chains can continue to be hydrolyzed to the corresponding molecules under specific pH conditions at the target site. These molecules can play a corresponding role at the target site. For example, the delivery molecule group can be the group corresponding to SN-38, which can provide SN-38 after hydrolysis, which overcomes the shortcomings of conventional hydrophobic antitumor drug delivery systems such as low drug loading capacity and strong side effects, thus improving drug safety and achieving the effect of killing cancer cells. In addition, the side chains can also be chemically connected to nucleic acid drugs or deliver nucleic acid drugs through physical action, forming a nano-formulation of nucleic acid drugs, which can significantly improve the in vivo stability of nucleic acid drugs. After the polymer particle is disintegrated, the delivery molecular groups can continue to be hydrolyzed (corresponding to chemical linkage) or released (corresponding to physical action delivery) into the corresponding nucleic acid drug molecules under specific pH conditions at the target site to exert the drug efficacy at the site of the lesion.

In a preferred embodiment, the chemical structural formula of the functionalized diblock copolymer is shown as one of the following:

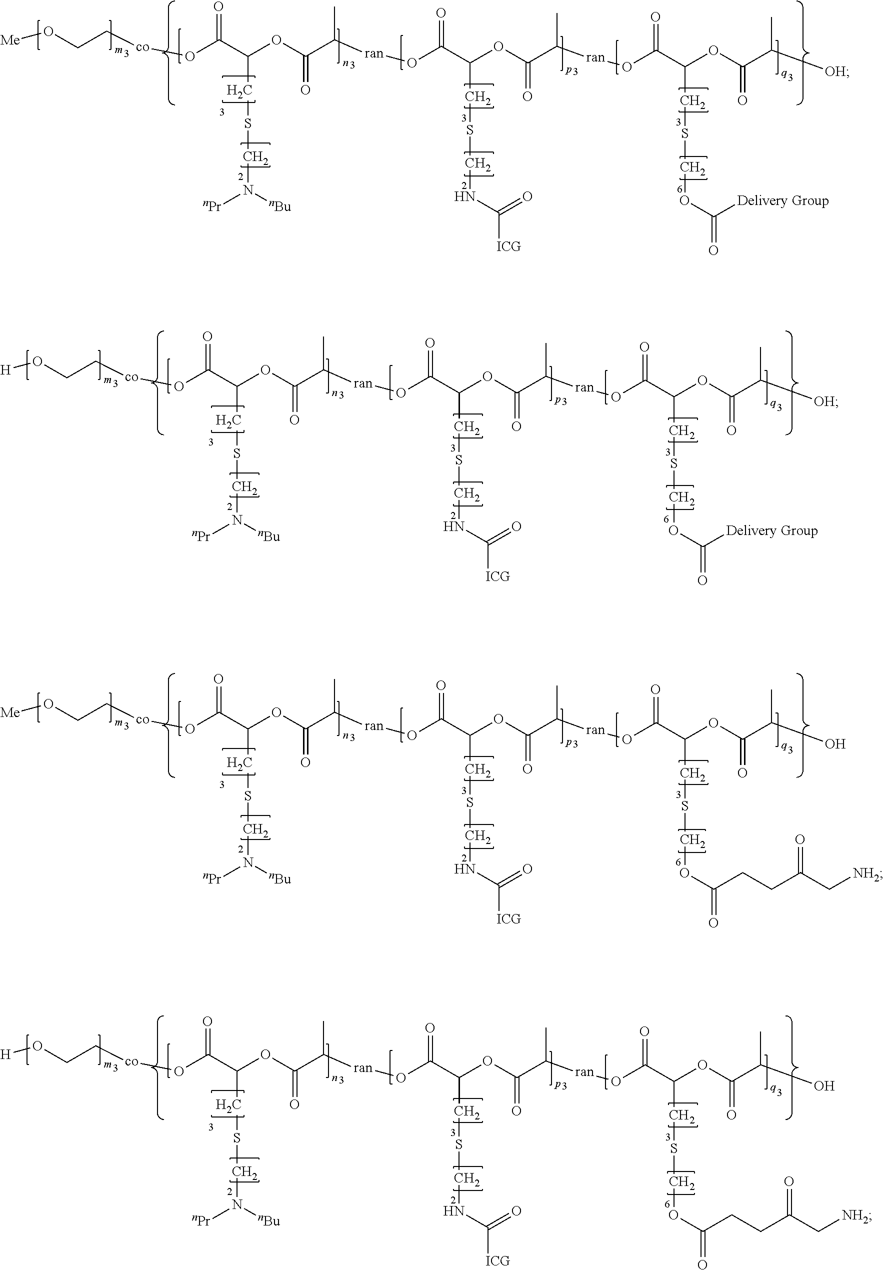

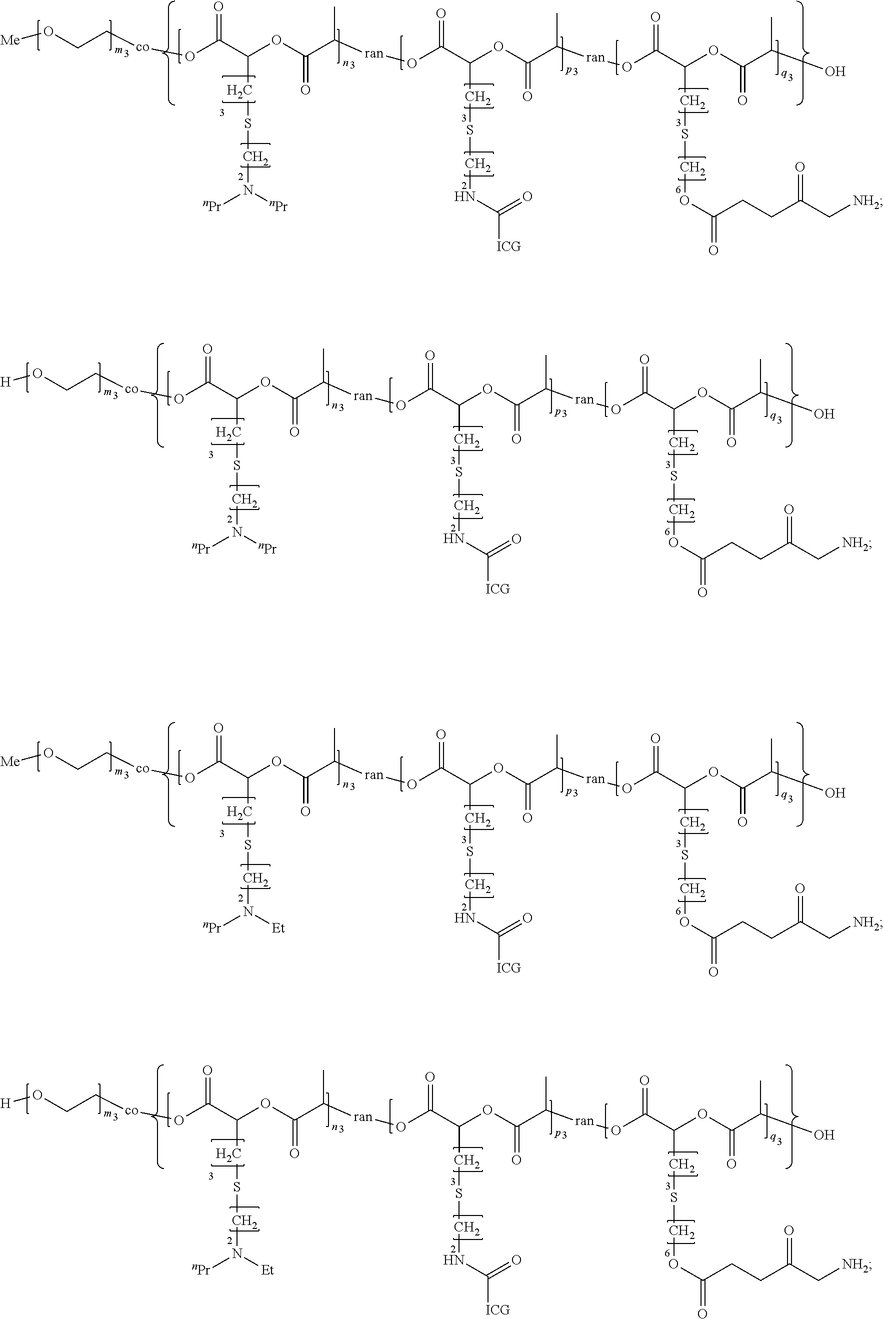

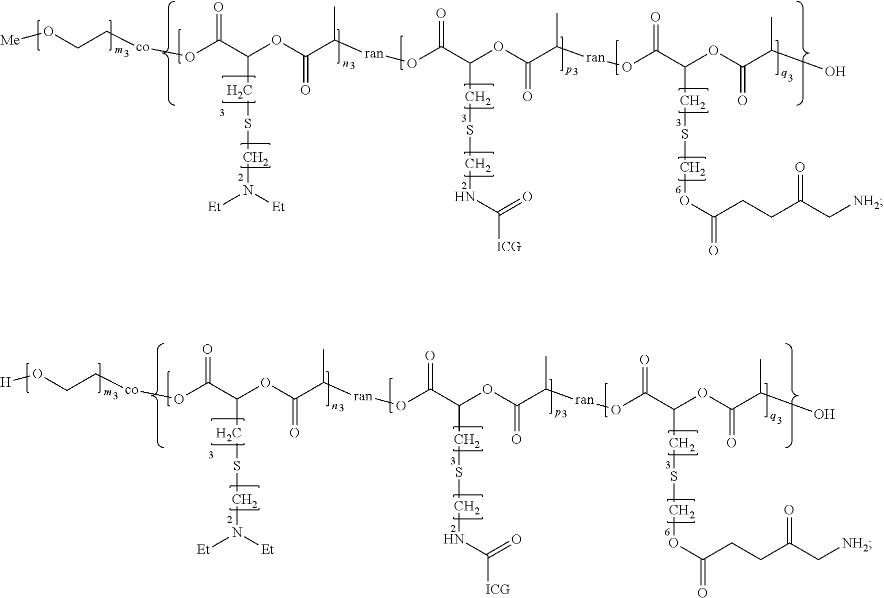

wherein m₃ is in a range of 22˜1136, preferably 44˜226, n₃ is in a range of 10˜500, preferably 30˜200, p₃ is in a range of 0.5˜5, and q₃ is in a range of 0˜500, preferably 10˜200.

In another preferred embodiment, m₃=44˜226, n₃=50˜200, p₃=0.5˜5, and q₃=50˜200.

The functionalized diblock copolymer provided in the present disclosure usually has a low critical micelle concentration (CMC), thereby reducing the difficulty of preparing polymer self-assembled particles, thereby ensuring that the prepared polymer particles have good stability in solution and blood. For example, the critical micelle concentration (CMC) of the functionalized diblock copolymer may be <50 μg/mL, <45 μg/mL, <40 μg/mL, <35 μg/mL, <30 μg/mL, <25 μg/mL, <20 μg/mL, <16 μg/mL, <14 μg/mL, <12 μg/mL, <10 μg/mL, ≤9 μg/mL, ≤8 μg/mL, ≤7 μg/mL, ≤6 μg/mL, ≤5 μg/mL, ≤4 μg/mL, or even smaller.

The second aspect of the present disclosure provides polymer particles prepared from the functionalized diblock copolymer provided in the first aspect of the present disclosure. The functionalized diblock copolymers described above can be used to form polymer particles. The fluorescent molecules distributed in the hydrophobic core of the polymer particles do not emit light under certain excitation conditions (for example, in the case of near-infrared ray as the excitation light source) due to the FRET effect. However, after being administered to an individual, the polymer particles can be enriched at the target site (for example, tumor site) through EPR passive targeting (or other tissue uptake methods). Because the target site has a special pH environment (for example, acidic environment), the protonatable group can be protonated in this pH range, and the charge repulsion generated by its protonation and the increase in polymer solubility drive the disintegration of the polymer particles. The FRET effect of the fluorescent group on each macromolecular segment is reduced or even completely eliminated. The polymer molecules in the dispersed state enriched at the target site can emit fluorescence under certain excitation conditions (for example, with the near-infrared ray as the excitation light source). For example, the aforementioned pH environment can be 6.5-6.8, which can correspond to the interstitial fluid of tumor tissue, and at least part of the polymer particles can reach the target site and stay in the interstitial fluid of the cells; for another example, the aforementioned pH environment can also be 4.5-6.5, which correspond to endosomes or lysosomes in tumor cells, and at least part of the polymer particles can interact with cells at the target site (for example, tumor cells) and enter into the cells through the endocytosis mechanism, thus reaching the above pH environment. The polymer particles prepared by the functionalized diblock copolymer provided in the present disclosure can be sufficiently diffused at the target site to achieve a clear fluorescence margin, and the functionalized diblock copolymer and/or polymer particles are bio-degradable in vivo. After being administered to an individual, polymer particles or nano-particles that fail to be targeted to the tumor site through the EPR effect cycle may be up-taken by the body's immune system (mainly macrophages, etc.) and then degraded (although PEG cannot be completely degraded in the body, PEG molecules with a molecular weight of less than 40,000 Da (for example, Roche's long-acting interferon, PEGASYS®, has been approved for safe clinical use for more than ten years, and its PEG has a molecular weight of 40,000 Da) can be effectively eliminated by the kidneys after circulating in the body; polylactide can be metabolized through the hydrolysis pathway, with a gradual decrease in molecular weight, and partly can be cleared by the kidneys). The polymer particles targeted to the target site through the EPR effect are disintegrated into free functionalized diblock copolymer molecules, which, under the pH conditions at the target site and the presence of a variety of enzymes, can be degraded into PEG (which can be cleared by the kidney after circulation) and degradable block (polylactide) polymers with gradually smaller molecular weights (which can be subsequently metabolized by circulation and partially cleared by the kidneys). These degradation pathways can improve the safety of the drug system for imaging probe applications or drug delivery system applications that are implemented (administered) in a single or multiple doses. Imaging observation results of live animals show that the block copolymer used can quickly achieve clear fluorescence imaging of tumor tissue after being injected into the living body. After about ten days of follow-up observation, it was found that the fluorescence presented at other sites (liver, kidney, pancreas, etc.) upon injection (the fluorescence appeared in these organs probably because some of the nanoparticles were captured by the reticuloendothelial system (RES) and then protonated by macrophages and other cells after the nanoparticles were phagocytosed, and finally disassembled into individual polymer chain segments) almost completely disappeared, providing strong evidence of the biodegradation and clearance performance of the present disclosure.

The polymer particles provided in the present disclosure can be nano-sized. For example, the particle size of the polymer particles can be 10˜200 nm, 10˜20 nm, 20˜30 nm, 30˜40 nm, 40˜60 nm, 60˜80 nm, 80 nm-100 nm, 100˜120 nm, 120˜140 nm, 140˜160 nm, 160˜180 nm, or 180˜200 nm.

In the polymer particles provided in the present disclosure, the polymer particles can also be modified with targeting groups, and these targeting groups can usually be modified on the surface of the polymer particles. Suitable methods for modifying the targeting group on the polymer particles should be known to those skilled in the art. For example, in general, the targeting group can be attached to the T end of the molecular structure of the functionalized diblock copolymer. These targeting groups can usually increase the efficiency of targeting nanoparticles to liver tumors based on the EPR effect (or other tissue uptake methods). These targeting groups can include, but are not limited to, various functional molecules such as (monoclonal) antibody fragments (for example, Fab, etc.), small molecule targeting groups (for example, folic acid, carbohydrates), polypeptide molecules (for example, cRGD, GL2P), and aptamers, and these functional molecules may have a targeting function (for example, a function of targeting tumor tissue). In a specific embodiment of the present disclosure, the targeting group is selected from -GalNac (N-acetylgalactosamine).

The third aspect of the present disclosure provides a method for preparing the polymer particles provided in the second aspect of the present disclosure. Based on the knowledge of the chemical structure of the functionalized diblock copolymer, a suitable method for forming polymer particles shall be known to those skilled in the art. For example, the method may include: dispersing an organic solvent including the above-mentioned functionalized diblock copolymer in water for self-assembling to provide the polymer particles; or conversely, dispersing water in an organic solvent including the functionalized diblock copolymer. In the above dispersion process, proper operations can be used to make the system fully mixed, for example, it can be carried out under ultrasonic conditions. For another example, the self-assembly process can usually be carried out by removing the organic solvent in the reaction system. The organic solvent removal method may specifically be a solvent volatilization method, an ultrafiltration method, and the like. For another example, the critical micelle concentration (CMC) of a polymer is related to the ratio of the hydrophobic block to the hydrophilic block of the polymer. The higher the ratio of the hydrophobic block, the smaller the CMC. When E1, E2, E3 are long-chain hydrophobic side chains, their content is inversely proportional to the value of CMC; when E1, E2, E3 are hydrophilic side chains, their content is directly proportional to the value of CMC. For another example, the particle size of polymer particles can usually be adjusted by extrusion instruments or microfluidic devices (homogenizers, NanoAssemblr and other devices).

The fourth aspect of the present disclosure provides the uses of the functionalized diblock copolymer provided in the first aspect of the present disclosure or the polymer particles provided in the second aspect of the present disclosure in the preparation of pharmaceutical preparations and/or reagents. Polymer nanoparticles formed as a drug delivery system allow the delivery of drugs or imaging probe molecules using polymer particles as carriers. As mentioned above, the products (for example, polymer particles) prepared by the functionalized diblock copolymers provided in the present disclosure have a passive targeting (enriched at the tumor sites through the general EPR effect of nanoparticles) or active targeting (enriched at the tumor sites by specific binding of nanoparticle surface-modified targeting groups to tumor surface-specific receptors) function. After administration to the individual, because the target site has a special pH environment (for example, acidic environment), the protonatable group can be protonated in this pH range. The charge repulsion generated by the protonation and the increase in polymer solubility drive the disintegration of polymer particles, and the FRET effect of the fluorescent group on the dispersed macromolecular segment is reduced or even completely eliminated, allowing the polymer molecules in the dispersed state enriched at the target site to emit fluorescence under certain excitation conditions (for example, in the case of near-infrared ray as the excitation light source). In this case, the target site (for example, the tumor site) can emit light specifically, which can be used for targeted imaging probing. In addition to imaging probe applications, these polymer particles can be used to prepare targeting agents. In a specific embodiment of the present disclosure, the above polymer particles can be used to prepare polymer particle-based drug delivery systems to deliver various drug molecules.

In the pharmaceutical preparations or reagents provided by the present disclosure, polymer particles can usually be used as carriers to deliver drugs or imaging probe molecules. The functionalized diblock copolymer can be used as a single active ingredient or can be combined with other active components to collectively form the active ingredient for the aforementioned uses.

The fifth aspect of the present disclosure provides a composition including the functionalized diblock copolymer provided in the first aspect of the present disclosure or the polymer particles provided in the second aspect of the present disclosure. As mentioned above, the aforementioned composition may be a targeting agent, and in a specific embodiment of the present disclosure, the aforementioned composition may be an imaging probe.

The composition provided in the present disclosure may also include at least one pharmaceutically acceptable carrier, which usually refers to a carrier for administration, which does not induce the production of antibodies harmful to the individual receiving the composition, and are not excessively toxic after administration. These carriers are well-known to those skilled in the art. For example, Remington's Pharmaceutical Sciences (Mack Pub. Co., N. J. 1991) discloses related content about pharmaceutically acceptable carriers. Specifically, the carrier may include one or more of saline, buffer, glucose, water, glycerol, ethanol, and adjuvant.

In the composition provided in the present disclosure, the functionalized diblock copolymer may serve as a single active ingredient, or may be combined with other active components for joint use. The other active components can be various other drugs and/or agents, which can usually act on the target site together with the above-mentioned functionalized diblock copolymer. The content of the active ingredient in the composition is usually a safe and effective amount, and the safe and effective amount should be adjustable for those skilled in the art. For example, the dosage of the active ingredient usually depends on the body weight of the subject to whom it is administered, the type of application, and the condition and severity of the disease.

The composition provided in the present disclosure can be adapted to any form of administration, for example, it can be pulmonary, nasal, rectal and/or intravenous injection, more specifically intradermal, subcutaneous, intramuscular, intraarticular, intraperitoneal, lung, oral, sublingual, nasal, percutaneous, vaginal, bladder instillation, uterine perfusion, intestinal perfusion, topical administration after craniotomy, or parenteral administration. Those skilled in the art can choose a suitable preparation form according to the mode of administration. For example, the preparation form suitable for parenteral administration may include, but is not limited to, a solution, a suspension, a reconstitutable dry preparation or a spray, etc. For another example, it may be in the form of an inhalant administered by inhalation.

The sixth aspect of the present disclosure provides a method of treatment or diagnosis, including: administering to an individual an effective amount of the functionalized diblock copolymer provided in the first aspect of the present disclosure, the polymer particles provided in the second aspect of the present disclosure, or the composition provided by the fifth aspect of the present disclosure. The “effective amount” generally refers to an amount that can achieve the desired effect after a proper administration period, for example, imaging, treatment of diseases, etc. The above-mentioned are pH-responsive and can be degraded under corresponding pH conditions. Chemical modifications on the functionalized diblock copolymers can also bring synergistic effect from delivery molecules, which are bonded to polymer molecules through degradable chemical bonds, and can be combined with a unique end group (targeting group, a group that can improve the immunogenicity of the system) to become a unique block copolymer-delivery bonded complex. In a specific embodiment of the present disclosure, after use, better intra-operative tumor boundary discrimination, and more precise removal of tumor lesions and metastatic tissue can be achieved. During the intra-operative imaging, the local delivery of molecules can be used to better kill cancer cells, reduce the recurrence rate, and improve the patient's post-operative survival.

The functionalized diblock copolymers or polymer particles provided in the present disclosure can significantly improve the safety of tumor imaging probe reagents and/or tumor drug preparations (tumor imaging probe reagents are mostly for single use; and tumor drug preparations are usually administered multiple times). For the diblock copolymer provided by the present disclosure (the compound of Formula III, i.e., PEG-PLA diblock copolymer), PEG can be safely removed from the human body (ADEGEN®, ONCASPAR®, etc., are clinically approved to use PEG with a molecular weight of 5K in multi-site modified therapeutic enzymes; biological macromolecules such as interferon, granulocyte colony stimulating factor, and antibody Fab fragments modified with 12-40K PEG have been safely used in clinical practice for more than ten years), another block PLGA can be gradually degraded under physiological conditions (hydrolysis; enzyme).

The functionalized diblock copolymers or polymer particles provided in the present disclosure can achieve high-quality imaging with tumor imaging probe reagents specific to solid tumor sites, and can be sensitive to pH changes at the tumor site (fluorescence signal changes ΔpH 10-90% only need about 0.2-0.3 pH unit), with high signal-to-noise ratio, clear boundary, and long half-life. Live imaging data show that the imaging probe used can have a long intratumoral retention and duration (several days or more) once enriched into the tumor, giving a longer observation window for tumor imaging surgery, and solving the problem of fluorescence imaging technology in real-time intra-operative navigation.

The functionalized diblock copolymers or polymer particles provided by the present disclosure can realize in vivo labeling of cancerous lymph nodes after administration, and lymph nodes with obvious fluorescence can be seen by in vivo imaging and in vitro anatomy. This finding is of great significance to the intra-operative determination of lymph node metastasis in future tumor resection surgery, and can have a significant positive impact on the prognosis and survival of patients. In addition to intravenous injection, the specific mode of administration can be local injection, such as local injection around areola or subcutaneous tissue in breast cancer resection, local injection in tissue in abdominal cavity in abdominal tumor surgery, and local subcutaneous or intramuscular injection in melanoma resection and treatment surgery.

The functionalized diblock copolymers, polymer particles, or compositions provided in the present disclosure can be conveniently administered locally, for example, bladder instillation, uterine perfusion, intestinal perfusion, local administration to the brain after craniotomy. The polymer particles used can be absorbed by the tumor tissue after sufficient contact with the tumor tissue, thereby achieving imaging and treatment of the tumor tissue.

The functionalized diblock copolymers or polymer particles provided in the present disclosure can introduce, based on the feature that the nanoparticles can diffuse sufficiently into the solid tumor microenvironment, precursor molecules (e.g., precursor molecules for photodynamic therapeutics, more specifically precursor molecules of 5-ALA) to the polymers, where these precursor molecules can be cleaved to the tumor microenvironment (weak acids, tumor microenvironment-specific proteases, etc.). The precursor molecules can be cleaved from the polymer backbone and converted to the clinically approved drug molecules (e.g., 5-ALA, etc.), enabling intra-operative image enhancement of tumor sites. At the same time as the implementation of imaging, the designed imaging probe reagent utilizes the light source of intra-operative imaging to realize the photodynamic therapy of tumor tissue during tumor resection surgery, reduce the damage of other photodynamic therapy on normal tissue, kill the uncut cancer tissue in the process of resection of the tumor tissue, reduce postoperative recurrence and prolong survival time.

In summary, the functionalized diblock copolymers or polymer particles provided in the present disclosure can be widely used in tumor imaging, tumor therapy and other fields. It not only has good safety, realizes faster and adjustable degradation and removal of polymers (by changing the number of functional groups) under acidic conditions, but also has excellent specific and high-quality imaging effects at the target site, with high signal-to-noise ratio, clear boundaries, long half-life, etc., which solves the problem of fluorescence imaging technology in real-time intra-operative navigation, and thus has a good industrialization prospect.

The following embodiments further illustrate the present disclosure, but do not limit the scope of the present disclosure.

The reaction route of the preparation method of the compound of formula III series in the embodiment is as follows:

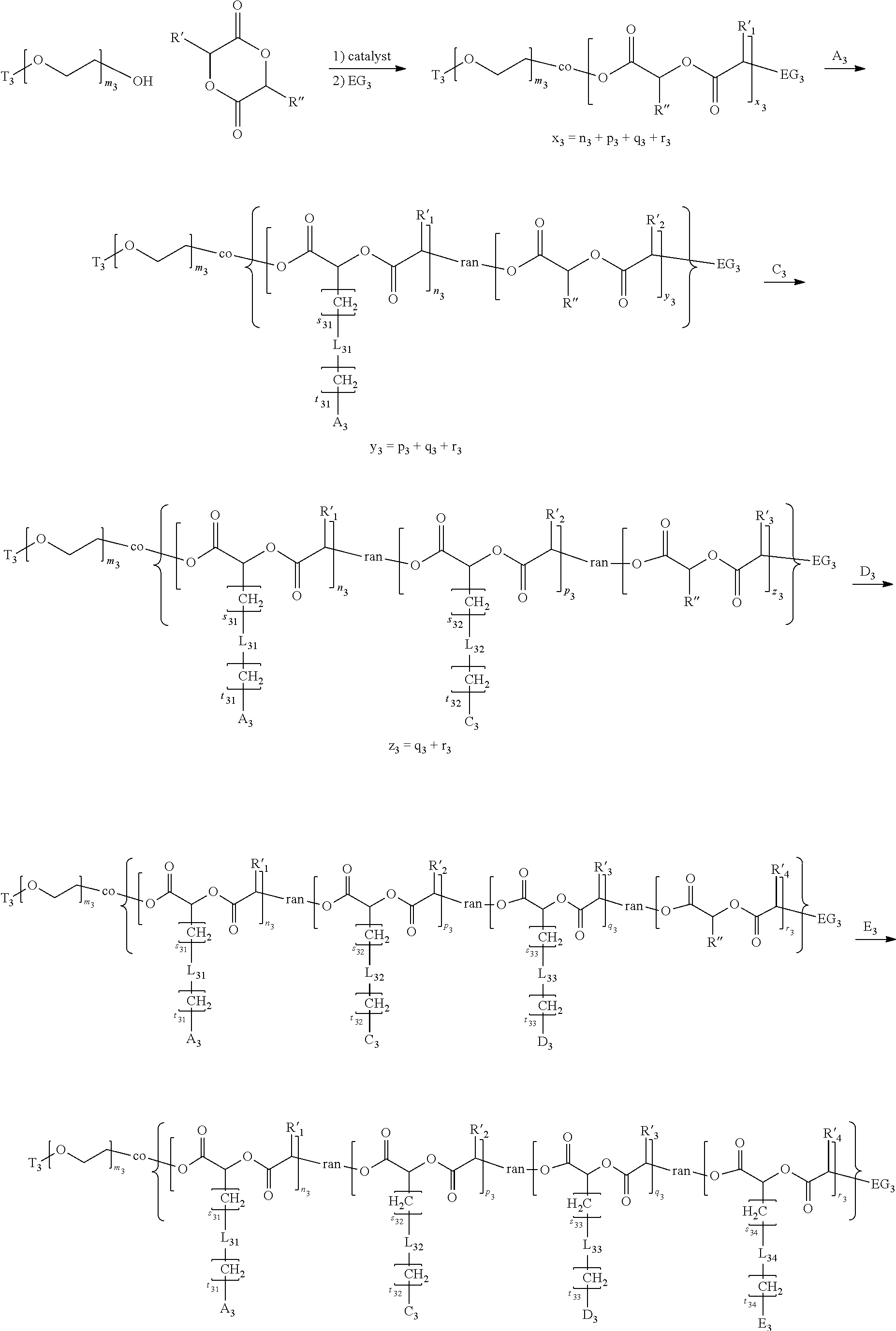

### EXAMPLE 1

### Synthesis of Monomer and Polymer of mPEG5k-PLA

### 1.1 Synthesis of Monomer

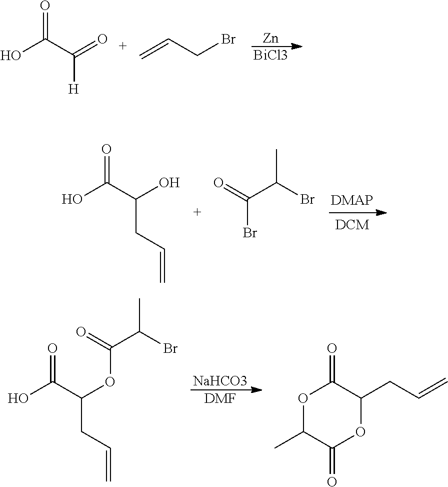

### Step 1: Synthesis of 2-hydroxypentyl-4-enoic acid (IB004-069-01)

Glyoxylic acid (3.7 g, 0.05 mol) was dissolved in 100 ml THF, and cooled to 0° C. in ice bath. Zinc particles (6.5 g, 0.1 mol) and BiCl₃ (22 g, 0.07 mol) were added respectively, and the reaction was performed under stirring at 0° C. for 3 hours, then 3-bromo-propylene (8.47 g, 0.07 mol) was added, and the reaction was continued overnight under Ar protection and stirring at room temperature. The reaction was quenched by adding 100 ml of 1 N HCl, filtered, and the filtrate was extracted with ethyl acetate (50 ml×3). The organic phases were combined, dried over anhydrous sodium sulfate, the solvent was removed by concentration, and the obtained product was dried to give 4.6 g crude product, which was used directly in the next step of the reaction without further purification. The yield is 80%. ¹H NMR (500 MHz, CDCl₃, ppm): δ 5.75-5.85 (m, 1H, CH═CH₂), 5.20 (m, 2H, CH₂═CH), 4.34 (m, 1H, HOCHCH₂, 2.46-2.64 (m, 2H, CH₂CH═CH₂).

### Step 2: Synthesis of 2-(2-bromo-propionyloxy)-pent-4-enoic acid (IB004-082-01)

DMAP (0.24 g, 0.002 mol) was dissolved in 50 ml of dichloromethane, protected by argon, cooled to 0° C. in an ice bath. 2-bromo-propionyl bromide (4.32 g, 0.02 mol) was added dropwise. 2-hydroxypentyl-4-enoic acid (2.32 g, 0.02 mol) and Et₃N (2.02 g, 0.02 mol) were dissolved in 10 ml of dichloromethane and then slowly added dropwise to the above solution. After completion of the dropwise addition, the reaction was performed overnight under stirring at room temperature. The solvent was removed by concentration, and the crude product was subjected to column separation and purification (EA:PE=1:15) to give a total of 963 mg of 2-(2-bromo-propionyloxy)-pent-4-enoic acid with the nature of a colorless transparent oil. The yield is 19%. ¹H NMR (500 MHz, CDCl₃, ppm): δ 1.86 (m, 3H, CH₃CHBr), 2.66-2.71 (m, 2H, CH₂CH═CH₂), 4.38-4.48 (m, 1H, CH₃CHBr), 5.18-5.24 (m, 3H, CH═CH₂ and HOOCCHOCO), 5.78-5.83 (m, 1H, CH═CH₂).

### Step 3: Synthesis of 3-allyl-6-methyl-[1,4]dioxane-2,5-dione (IB004-084-01)

NaHCO₃ (181 mg, 1.72 mmol) was dispersed in 10 ml of DMF, the system was protected under argon. 2-(2-bromo-propionyloxy)-pent-4-enoic acid (963 mg, 3.8 mmol) was dissolved in 5 ml of DMF, and then slowly added dropwise to the above reaction solution. After completion of the dropwise addition, the reaction was performed overnight under stirring at room temperature. DMF was removed by concentration, and the crude product was subjected to column separation and purification (EA:PE=1:10), to give a total of 200 mg of 3-allyl-6-methyl-[1,4]dioxane-2,5-dione with the nature of a colorless and transparent oil. The yield is 30.6%. ¹H NMR (500 MHz, CDCl₃, ppm): δ 1.67-1.71 (m, 3H, CH₃CH), 2.72-2.86 (m, 2H, CH₂CH═CH₂), 4.95-5.01 (m, 1H, CHCH₂CH═CH₂), 5.02-5.09 (m, 1H, CH₃CH), 5.23-5.33 (m, 2H, CH₂CH═CH₂), 5.83-5.88 (m, 1H, CH₂CH═CH₂).

### 1.2 Polymerization

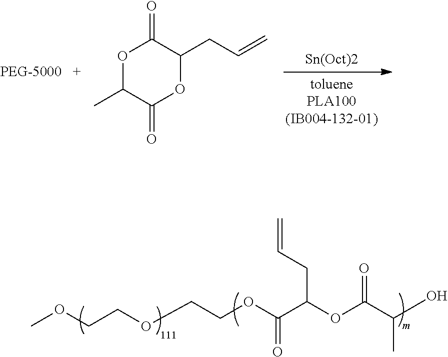

In a glove box with H₂O and O₂ indicators less than 0.1 ppm, m-PEG-5000 (50 mg, 0.01 mmol) and 3-allyl-6-methyl-[1,4]dioxane-2,5-dione (170 mg, 1 mmol) were weighed and placed into a polymerization reaction tube. Sn(Oct)₂ (40.5 mg, 0.1 mmol) was dissolved in 200 μL of toluene, and 20 μL of it was added into the polymerization reaction tube. The polymerization reaction tube was sealed, removed from the glove box, heated to 130° C., and stirred for 1 h for reaction. The reaction system was cooled to room temperature, 1 ml of DCM was added and then dissolved under stirring, The reaction system was transferred to a 100 ml eggplant shaped flask, 50 ml of methyl tert-butyl ether was added slowly under stirring, and then a white solid was precipitated. The stirring was continued for 10 min and then stopped, the methyl tert-butyl ether was poured out, and the residual solids were dried by an oil pump to give a total of 187 mg of the polymer, which is in the nature of a white solid. The yield is 85%.

### EXAMPLE 2

### General Method for Modification of Hydrophilic/Hydrophobic Groups

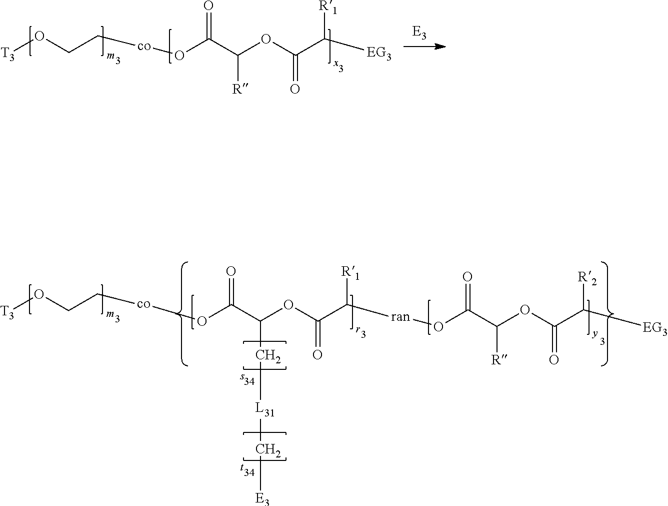

The polymer to be modified was added into the photochemical reactor, and solvent such as dichloromethane or water is selected according to the solubility of the polymer, and the reaction system should be completely dissolved. The reaction concentration was 100 mg polymer/1000 μL. The hydrophilic/hydrophobic modifying group, 0.2 mol equivalent amount of 2,2-dimethoxy-2-phenylacetophenone were added, and the reaction solution reacted under 365 nm UV light irradiation for 2 hours at room temperature. After the reaction, the reaction solution was concentrated. The precipitate was washed with n-heptane. The polymer precipitate was obtained after filtration, and the product is a white solid.

### EXAMPLE 3

### General Method for Modification of Protonatable Groups

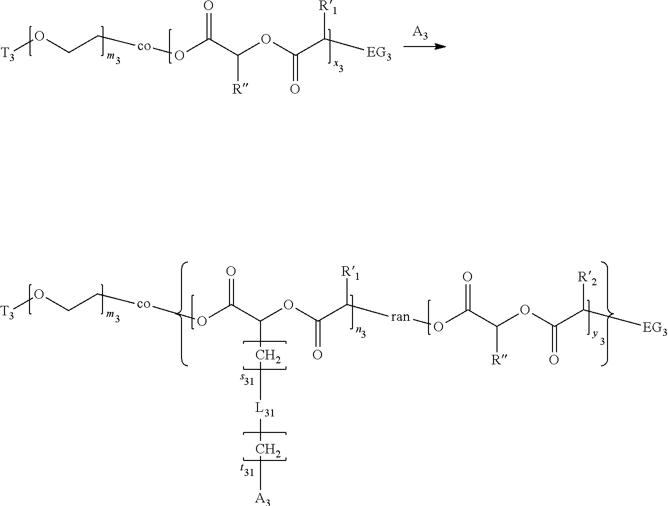

The polymer to be modified was added into the photochemical reactor, and solvent such as dichloromethane or water is selected according to the solubility of the polymer, and the reaction system should be completely dissolved. The reaction concentration was 100 mg polymer/1000 μL. The protonatable modifying group, 0.2 mol equivalent amount of 2,2-dimethoxy-2-phenylacetophenone were added, and the reaction solution reacted under 365 nm UV light irradiation for 2 hours at room temperature. The reaction was monitored using NMR until it was completed. After the reaction, the reaction solution was concentrated. The precipitate was washed with n-heptane. The polymer precipitate was obtained after filtration, and the product is a white solid.

### EXAMPLE 4

### General Method for Modification of Fluorescent Groups

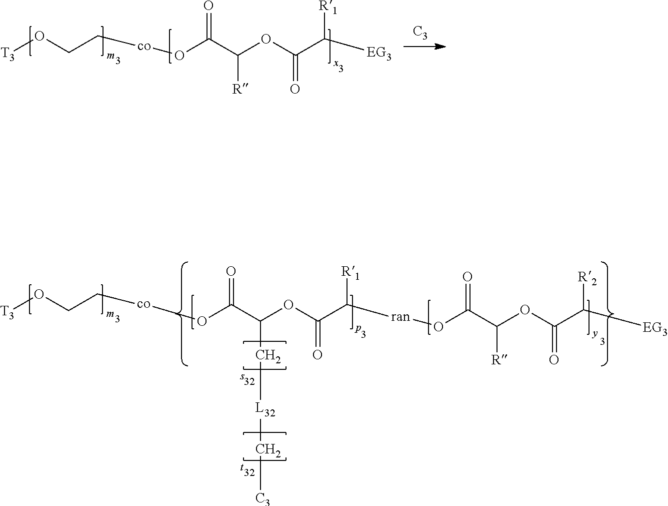

The polymer to be modified was added into a flask, and solvent such as dichloromethane or water is selected according to the solubility of the polymer, and the reaction system should be completely dissolved. The reaction concentration was 50 mg polymer/1000 μL. The fluorescent modifying group was added, and the reaction was performed over night at room temperature. After the reaction, the reaction solution was concentrated. The precipitate was dissolved in ethanol. The solution was dialyzed with dialysis bag to remove small molecules. The polymer obtained by concentration is a green solid.

### EXAMPLE 5

### General Method for Modification of Delivery Molecular Groups

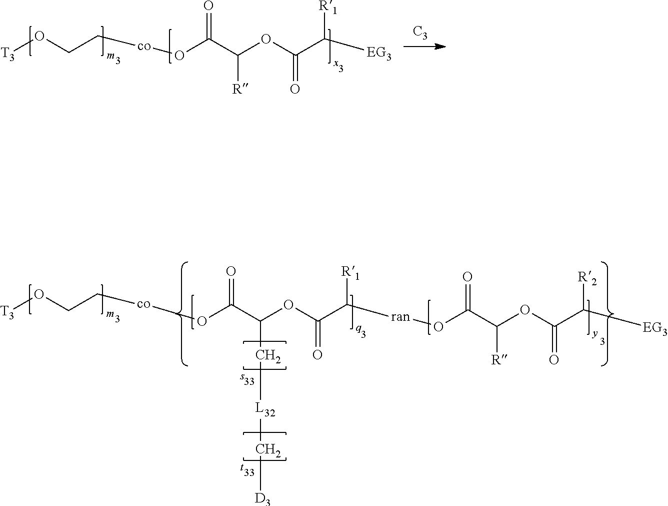

The polymer to be modified was added into the photochemical reactor, and solvent such as dichloromethane or water is selected according to the solubility of the polymer, and the reaction system should be completely dissolved. The reaction concentration was 100 mg polymer/1000 μL. The delivery molecular modifying group, 0.2 mol equivalent amount of 2,2-dimethoxy-2-phenylacetophenone were added, and the reaction solution reacted under 365 nm UV light irradiation for 2 hours at room temperature. The reaction was monitored using NMR until the reaction was completed. After the reaction, the reaction solution was concentrated. The precipitate was washed with n-heptane. The polymer precipitate was obtained after filtration, and the product is a white solid.

### EXAMPLE 6

### Example Series Containing N-Nonyl Group

### 6.1 PLA110-C9-TEE-ICG

### 6.1.1 Synthetic Route of PLA110-C9-TEE-ICG

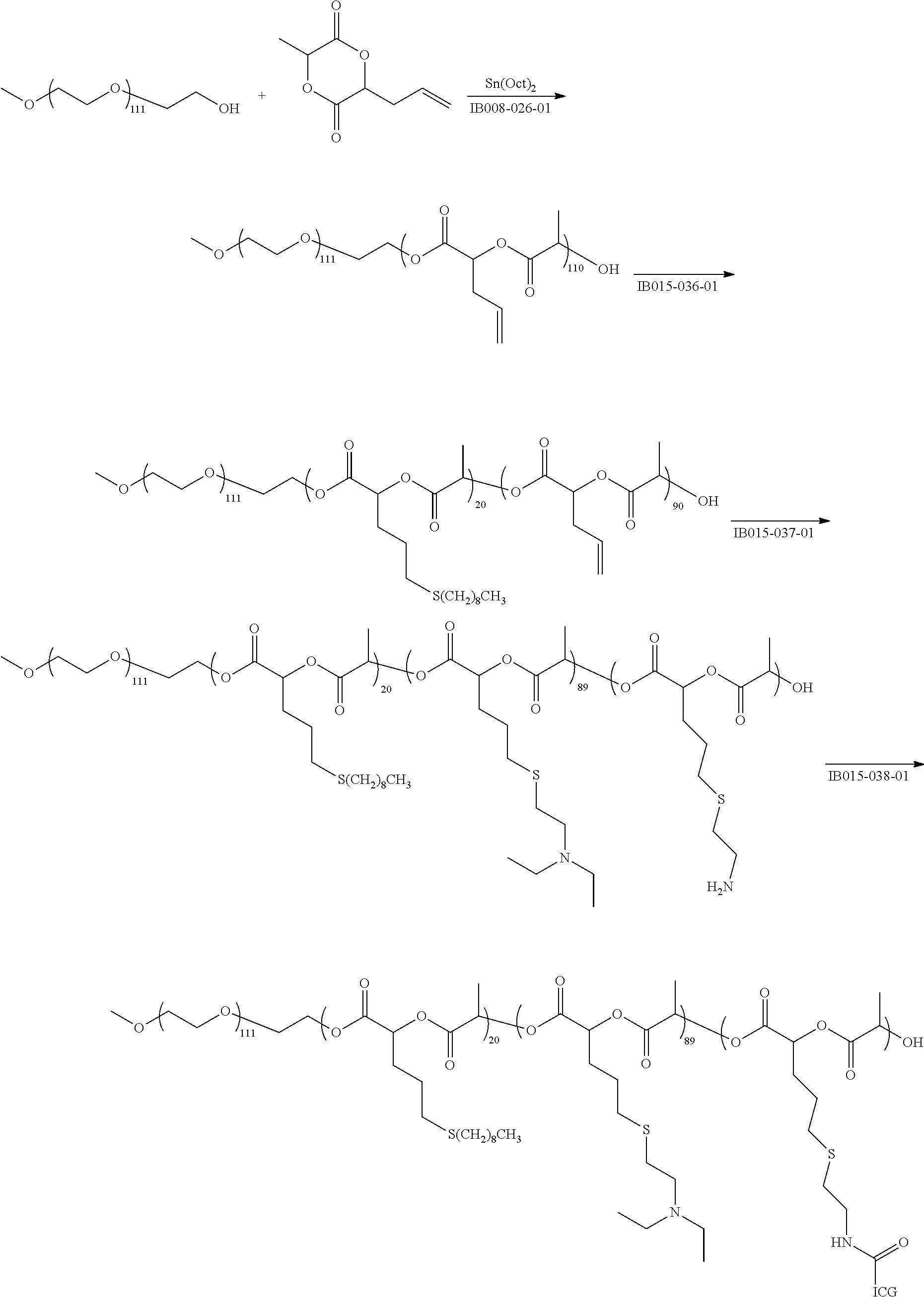

### 6.1.2 Synthesis of PLA110 (IB008-026-01)

The synthesis and purification of Example 6.1.2 was carried out according to the procedure in Example 1.2 above (using 1.02 g of 3-allyl-6-methyl-[1,4]dioxane-2,5-dione, 6 mmol) to give a total of 1.16 g of white solid polymer in 91.3% yield. ¹H NMR (400 MHz, Chloroform-d) δ 5.85-5.67 (m, 109H), 5.28-5.08 (m, 452H), 3.63 (s, 448H), 2.67 (m, 240H), 1.55 (m, 343H).

### 6.1.3 Synthesis of PLA110-C9 (IB008-036-01)

The synthesis and purification of Example 6.1.3 was carried out according to the procedure in Example 2 above (using dichloromethane as the solvent) to give a total of 52 mg of white solid polymer in 91% yield. ¹H NMR (400 MHz, Chloroform-d) δ 5.85-5.67 (m, 89H), 5.28-5.08 (m, 432H), 3.63 (d, J=1.2 Hz, 448H), 2.75-2.56 (m, 220H), 2.46-2.39 (m, 41H), 1.90-1.78 (m, 40H), 1.61-1.44 (m, 447H), 1.29-1.26 (m, 210H), 0.89-0.87 (m, 62H).

### 6.1.4 Synthesis of PLA110-C9-TEE (IB008-037-01)

The synthesis and purification of Example 6.1.4 was carried out according to the procedure in Example 3 above (using dichloromethane as the solvent) to give a total of 65 mg of white solid polymer in 89% yield. ¹H NMR (400 MHz, Chloroform-d) δ 5.14 (s, 332H), 3.60 (d, J=1.2 Hz, 448H), 3.10-2.57 (m, 1238H), 1.90-1.78 (m, 241H), 1.58-1.43 (m, 549H), 0.92-0.89 (m, 59H).

### 6.1.5 Synthesis of PLA110-C9-TEE-ICG (IB008-038-01)

The synthesis and purification of Example 6.1.5 was carried out according to the procedure in Example 4 above (using dichloromethane as the solvent) to give a total of 32 mg of green solid polymer in 76% yield. ¹H NMR (400 MHz, Chloroform-d) δ 8.12-7.31 (m, 19 H), 5.14 (s, 322H), 3.62 (d, J=1.2 Hz, 448H), 3.09-2.58 (m, 1207H), 1.90-1.77 (m, 238H), 1.57-1.44 (m, 579H), 1.29-1.26 (m, 218H), 0.89-0.86 (m, 63H).

### EXAMPLE 7

### Example Series Containing Octadecyl Group

### 7.1 PLA110-C18-TEE-ICG

### 7.1.1 Synthetic Route of PLA110-C18-TEE-ICG

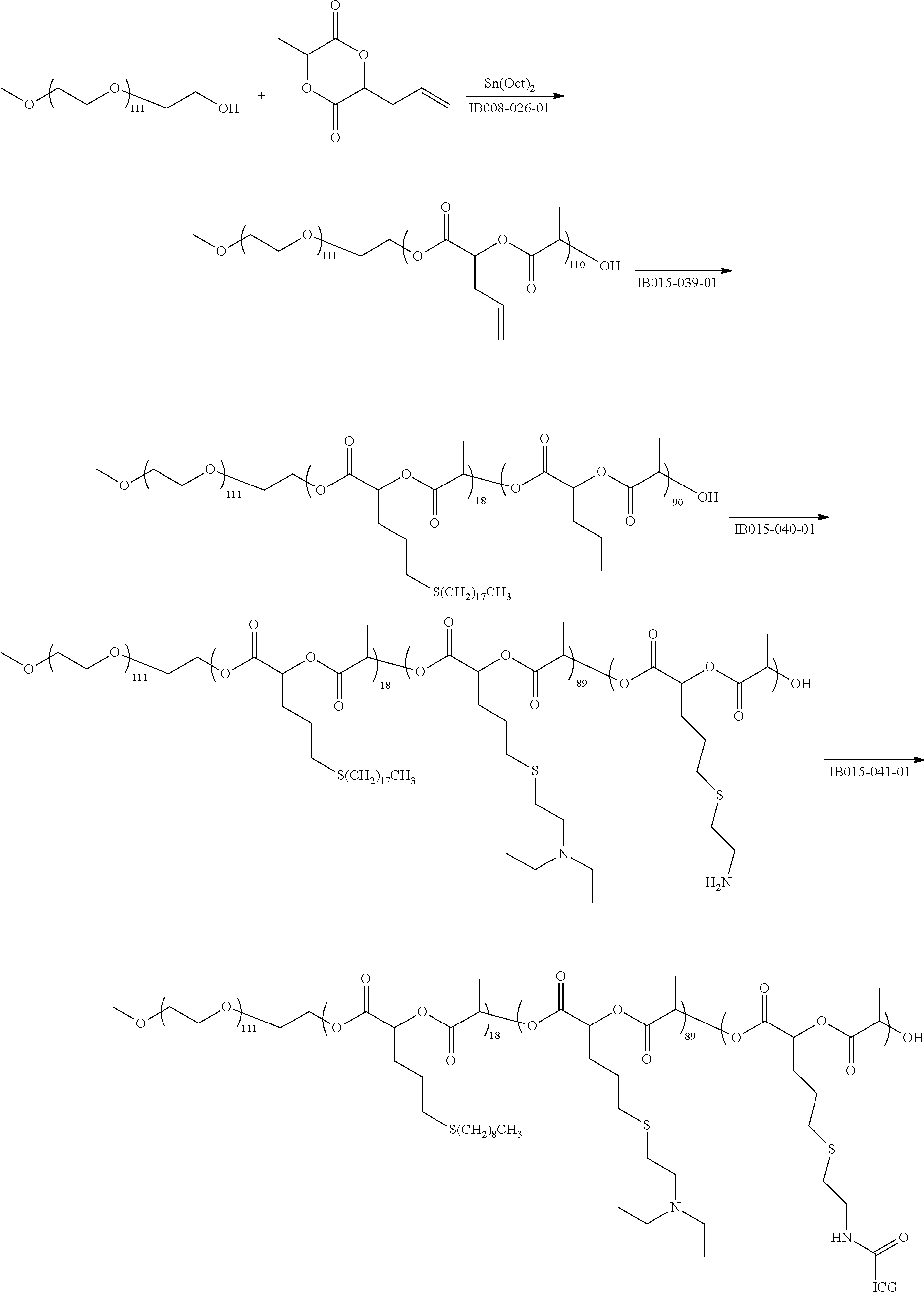

### 7.1.2 Synthesis of PLA110 (IB008-026-01)

The synthesis and purification of Example 7.1.2 was carried out according to the procedure in Example 1.2 above (using 1.02 g of 3-allyl-6-methyl-[1,4]dioxane-2,5-dione, 6 mmol) to give a total of 1.16 g of white solid polymer in 91.3% yield. ¹H NMR (400 MHz, Chloroform-d) δ 5.84-5.66 (m, 109H), 5.28-5.08 (m, 452H), 3.62 (s, 448H), 2.67 (m, 240H), 1.54 (m, 343H).

### 7.1.3 Synthesis of PLA110-C18 (IB015-039-01)

The synthesis and purification of Example 7.1.3 was carried out according to the procedure in Example 2 above (using dichloromethane as the solvent) to give a total of 56 mg of white solid polymer in 93% yield. ¹H NMR (400 MHz, Chloroform-d) δ 5.85-5.67 (m, 89H), 5.30-5.07 (m, 432H), 3.63 (d, J=1.2 Hz, 448H), 2.75-2.56 (m, 208H), 2.46-2.38 (m, 40H), 1.90-1.76 (m, 39H), 1.58-1.44 (m, 442H), 1.29-1.24(m, 565H), 0.89-0.87 (m, 58H).

### 7.1.4 Synthesis of PLA110-C18-TEE (IB015-040-01)

The synthesis and purification of Example 7.1.4 was carried out according to the procedure in Example 3 above (using dichloromethane as the solvent) to give a total of 60 mg of white solid polymer in 85% yield. ¹H NMR (400 MHz, Chloroform-d) δ 5.13 (s, 337H), 3.63 (d, J=1.2 Hz, 448H), 3.12-2.57 (m, 1122H), 2.44-2.38 (m, 41H), 1.91-1.76 (m, 239H), 1.57-1.44 (m, 543H), 0.89-0.87 (m, 61H).

### 7.1.5 Synthesis of PLA110-C18-TEE-ICG (IB015-041-01)

The synthesis and purification of Example 7.1.5 was carried out according to the procedure in Example 4 above (using dichloromethane as the solvent) to give a total of 33 mg of green solid polymer in 72% yield. ¹H NMR (400 MHz, Chloroform-d) δ 8.14-7.30 (m, 27 H), 5.14 (s, 327H), 3.63 (d, J=1.2 Hz, 448H), 3.09-2.58 (m, 1172H), 2.47-2.39 (m, 42H), 1.90-1.78 (m, 240H), 1.57-1.44 (m, 543H), 0.89-0.87 (m, 61H).

### EXAMPLE 8

### Example Series Containing N-Butyl Group

### 8.1 PLA110-C4-TEE-ICG

### 8.1.1 Synthetic Route of PLA110-C4-TEE-ICG

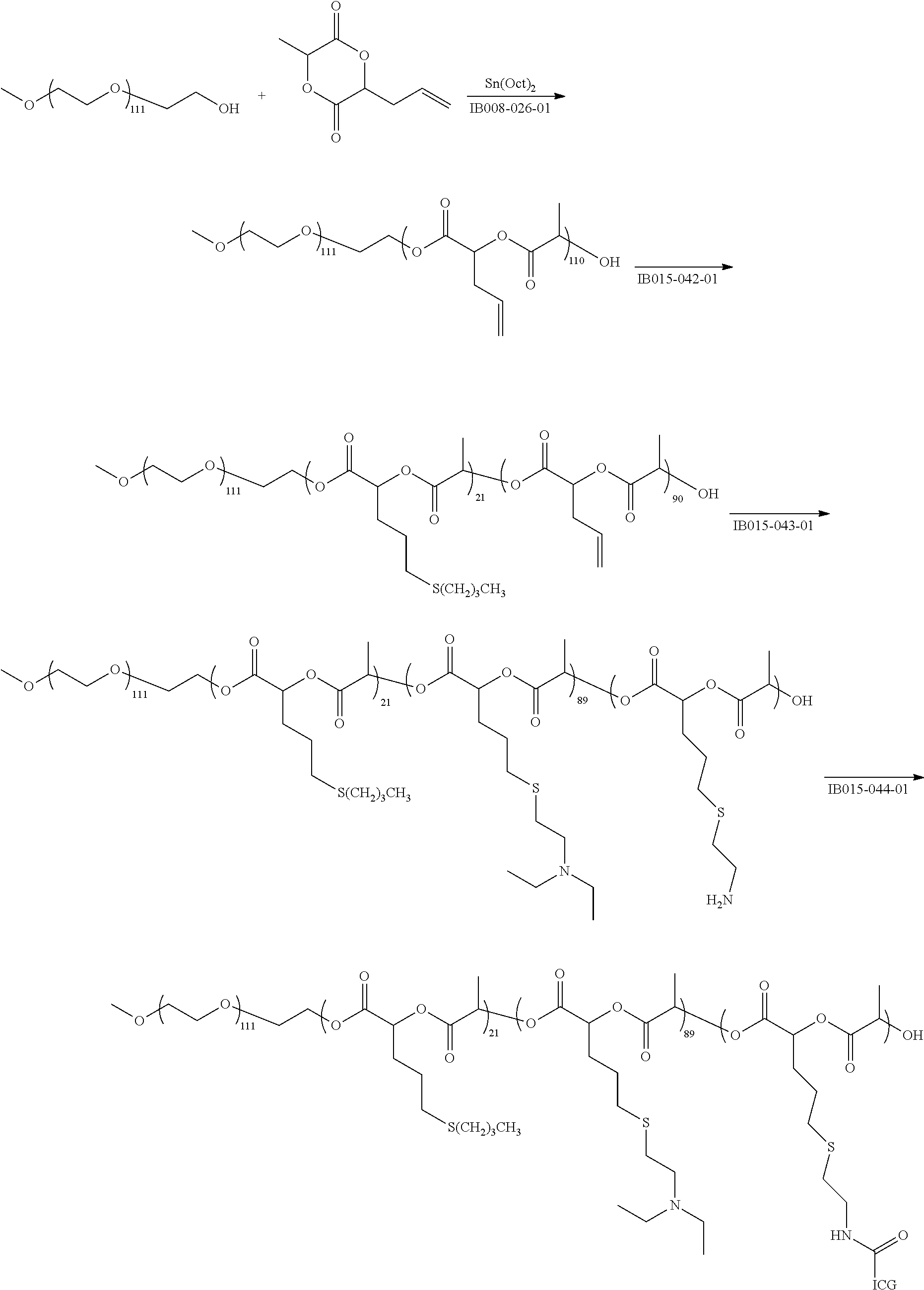

### 8.1.2 Synthesis of PLA110 (IB008-026-01)

The synthesis and purification of Example 8.1.2 was carried out according to the procedure in Example 1.2 above (using 1.02 g of 3-allyl-6-methyl-[1,4]dioxane-2,5-dione, 6 mmol) to give a total of 1.16 g of white solid polymer in 91.3% yield. ¹H NMR (400 MHz, Chloroform-d) δ 5.85-5.67 (m, 109H), 5.28-5.08 (m, 452H), 3.63 (s, 448H), 2.67 (m, 240H), 1.55 (m, 343H).

### 8.1.3 Synthesis of PLA110-C4 (IB015-042-01)

The synthesis and purification of Example 8.1.3 was carried out according to the procedure in Example 2 above (using dichloromethane as the solvent) to give a total of 51 mg of white solid polymer in 89% yield. ¹H NMR (400 MHz, Chloroform-d) δ 5.85-5.67 (m, 89H), 5.28-5.08 (m, 432H), 3.63 (d, J=1.2 Hz, 448H), 2.75-2.56 (m, 223H), 2.46-2.39 (m, 38H), 1.90-1.78 (m, 40H), 1.61-1.44 (m, 442H), 0.92-0.89 (m, 61H).

### 8.1.4 Synthesis of PLA110-C4-TEE (IB015-043-01)

The synthesis and purification of Example 8.1.4 was carried out according to the procedure in Example 3 above (using dichloromethane as the solvent) to give a total of 54 mg of white solid polymer in 91% yield. ¹H NMR (400 MHz, Chloroform-d) δ 5.14 (s, 332H), 3.63 (s, 448H), 3.09-2.58 (m, 1238H), 1.90-1.78 (m, 240H), 1.57-1.44 (m, 549H), 0.92-0.89 (m, 60H)).

### 8.1.5 Synthesis of PLA110-C4-TEE-ICG (IB015-044-01)

The synthesis and purification of Example 8.1.5 was carried out according to the procedure in Example 4 above (using dichloromethane as the solvent) to give a total of 36 mg of green solid polymer in 75% yield. ¹H NMR (400 MHz, Chloroform-d) δ 8.09-7.28 (m, 24H), 5.14 (s, 333H), 3.63 (s, 448H), 3.07-2.56 (m, 1238H), 1.91-1.80 (m, 243H), 1.58-1.43 (m, 548H), 0.92-0.88 (m, 59H).

### EXAMPLE 9

### Example Series Containing Ethyl Group

### 9.1 PLA110-C2-TEE-ICG

### 9.1.1 Synthetic Route of PLA110-C2-TEE-ICG

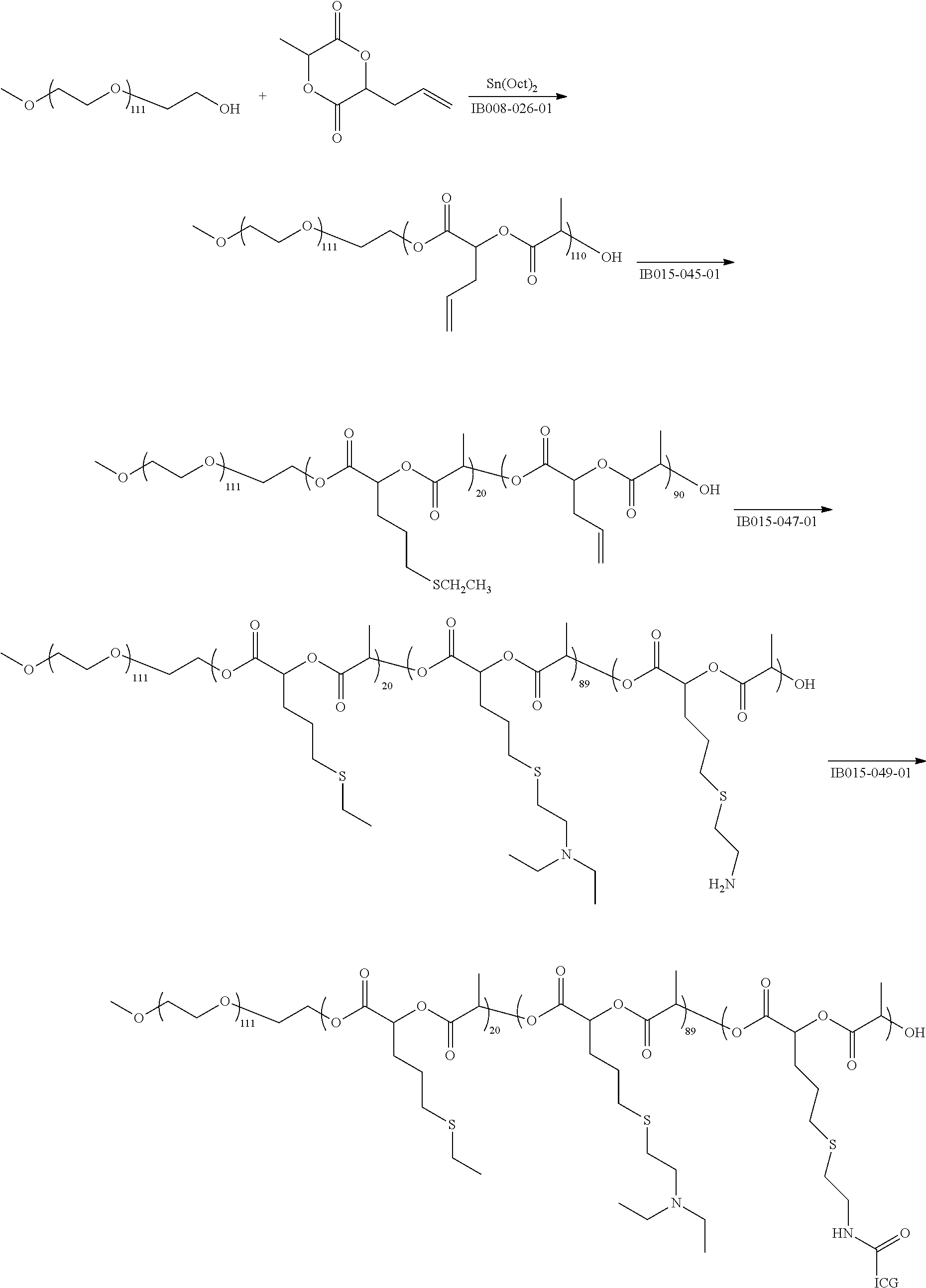

### 9.1.2 Synthesis of PLA110 (IB008-026-01)

The synthesis and purification of Example 9.1.2 was carried out according to the procedure in Example 1.2 above (using 1.02 g of 3-allyl-6-methyl-[1,4]dioxane-2,5-dione, 6 mmol) to give a total of 1.16 g of white solid polymer in 91.3% yield. ¹H NMR (400 MHz, Chloroform-d) δ 5.85-5.68 (m, 109H), 5.28-5.08 (m, 452H), 3.62 (s, 448H), 2.67 (m, 240H), 1.56 (m, 343H).

### 9.1.3 Synthesis of PLA110-C2 (IB015-045-01)

The synthesis and purification of Example 9.1.3 was carried out according to the procedure in Example 2 above (using dichloromethane as the solvent) to give a total of 55 mg of white solid polymer in 89% yield. ¹H NMR (400 MHz, Chloroform-d) δ 5.85-5.67 (m, 89H), 5.28-5.08 (m, 432H), 3.63 (d, J=1.2 Hz, 448H), 2.75-2.56 (m, 208H), 2.50-2.46 (m, 42H), 1.90-1.78 (m, 40H), 1.61-1.55 (m, 363H), 1.17-1.12 (m, 63H).

### 9.1.4 Synthesis of PLA110-C2-TEE (IB015-047-01)

The synthesis and purification of Example 9.1.4 was carried out according to the procedure in Example 3 above (using dichloromethane as the solvent) to give a total of 64 mg of white solid polymer in 91% yield. ¹H NMR (400 MHz, Chloroform-d) δ, 5.14 (s, 322H), 3.63 (d, J=1.2 Hz, 448H), 3.09-2.58 (m, 1198H), 2.50-2.46 (m, 42H), 1.90-1.78 (m, 240H), 1.55 (m, 473H), 1.17-1.13 (m, 63H).

### 9.1.5 Synthesis of PLA110-C2-TEE-ICG (IB015-049-01)

The synthesis and purification of Example 9.1.5 was carried out according to the procedure in Example 4 above (using dichloromethane as the solvent) to give a total of 40 mg of green solid polymer in 75% yield. ¹H NMR (400 MHz, Chloroform-d) δ, 8.08-7.28 (m, 20H), 5.14 (s, 320H), 3.63 (d, J=1.2 Hz, 448H), 3.09-2.58 (m, 1198H), 2.50-2.46 (m, 42H), 1.90 -1.78 (m, 240H), 1.55 (m, 473H), 1.17-1.13 (m, 63H).

### EXAMPLE 10

### Example Series Containing Cholesterol Groups

### 10.1 PLA110-CHOL-TEE-ICG

### 10.1.1 Synthetic Route of PLA110-CHOL-TEE-ICG

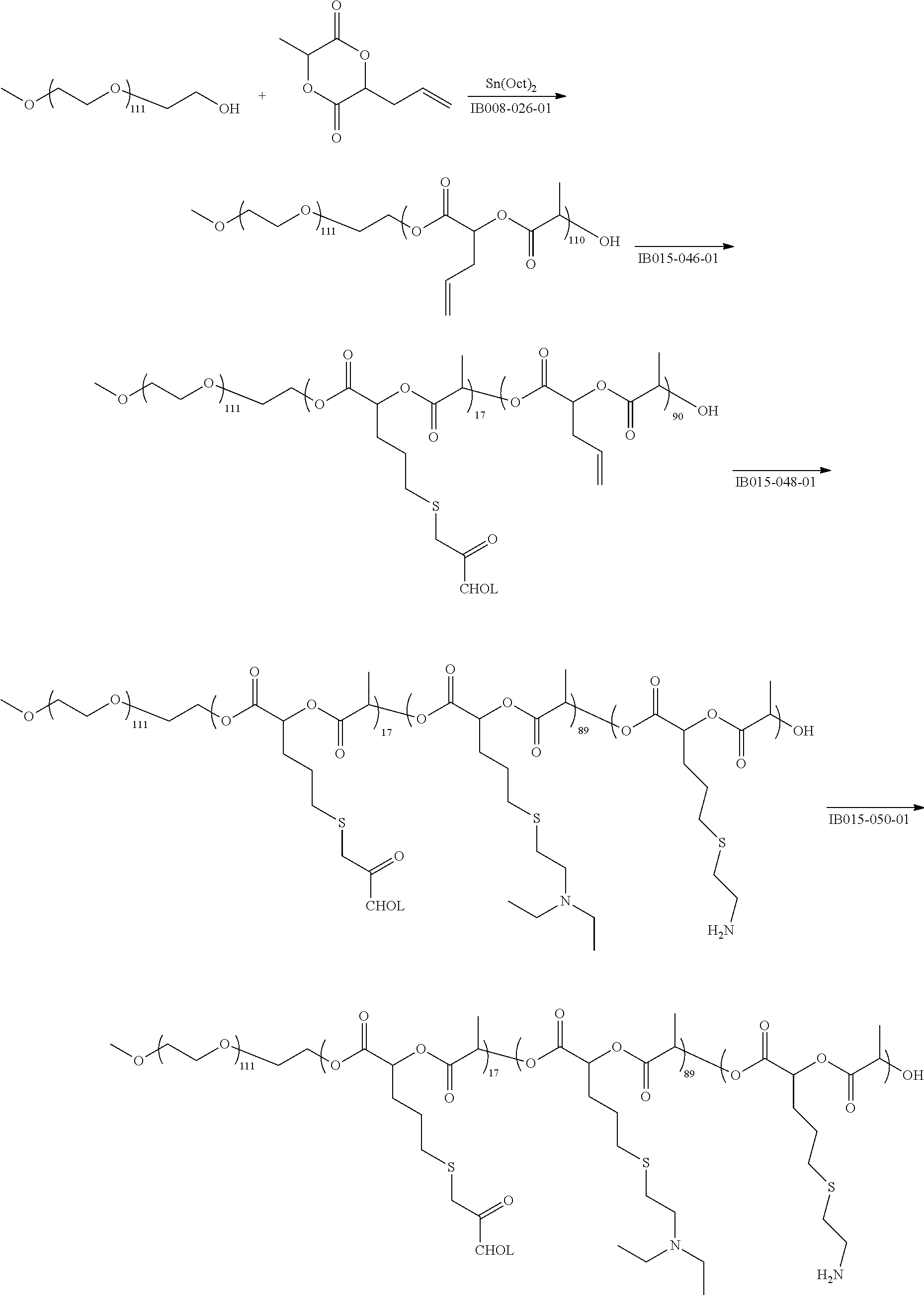

### 10.1.2 Synthesis of PLA110 (IB008-026-01)

The synthesis and purification of Example 10.1.2 was carried out according to the procedure in Example 1.2 above (using 1.02 g of 3-allyl-6-methyl-[1,4]dioxane-2,5-dione, 6 mmol) to give a total of 1.16 g of white solid polymer in 91.3% yield. ¹H NMR (400 MHz, Chloroform-d) δ 5.85-5.67 (m, 109H), 5.28-5.08 (m, 452H), 3.63 (s, 448H), 2.67 (m, 240H), 1.55 (m, 343H).

### 10.1.3 Synthesis of PLA110-CHOL (IB015-046-01)

The synthesis and purification of Example 10.1.3 was carried out according to the procedure in Example 2 above (using dichloromethane as the solvent) to give a total of 61 mg of white solid polymer in 93% yield. ¹H NMR (400 MHz, Chloroform-d) δ 5.85-5.67 (m, 89H), 5.28-5.08 (m, 451H), 4.40 (d, J=9.8 Hz, 2H), 3.63 (d, J=1.2 Hz, 448H), 3.02 (d, J=8.2 Hz, 20H), 2.75-2.56 (m, 220H), 2.12 (d, J=8.3 Hz, 20H), 1.90-0.58 (m, 1260H), 0.46 (s, 61H).

### 10.1.4 Synthesis of PLA110-CHOL-TEE (IB015-048-01)

The synthesis and purification of Example 10.1.4 was carried out according to the procedure in Example 3 above (using dichloromethane as the solvent) to give a total of 75 mg of white solid polymer in 92% yield. ¹H NMR (400 MHz, Chloroform-d) δ 5.21-5.11 (m, 352H), 4.40 (d, J=9.8 Hz, 20H), 3.63 (d, J=1.2 Hz, 448H), 3.09-2.58 (m, 1231H), 2.12 (d, J=8.3 Hz, 42H), 1.90-0.58 (m, 1588H), 0.46 (s, 63H).

### 10.1.5 Synthesis of PLA110-CHOL-TEE-ICG (IB015-050-01)

The synthesis and purification of Example 10.1.5 was carried out according to the procedure in Example 4 above (using dichloromethane as the solvent) to give a total of 43 mg of green solid polymer in 85% yield. ¹H NMR (400 MHz, Chloroform-d) δ 8.12-7.28 (m, 21 H), 5.21-5.11 (m, 352H), 4.40 (d, J=9.8 Hz, 20H), 3.63 (d, J=1.2 Hz, 448H), 3.09-2.58 (m, 1231H), 2.12 (d, J=8.3 Hz, 42H), 1.90-0.58 (m, 1588H), 0.46 (s, 63H).

### EXAMPLE 11

### Example Series Containing Hydroxyethyl Group

### 11.1 PLA110-C2OH-TEE-ICG

### 11.1.1 Synthetic Route of PLA110-C2OH-TEE-ICG

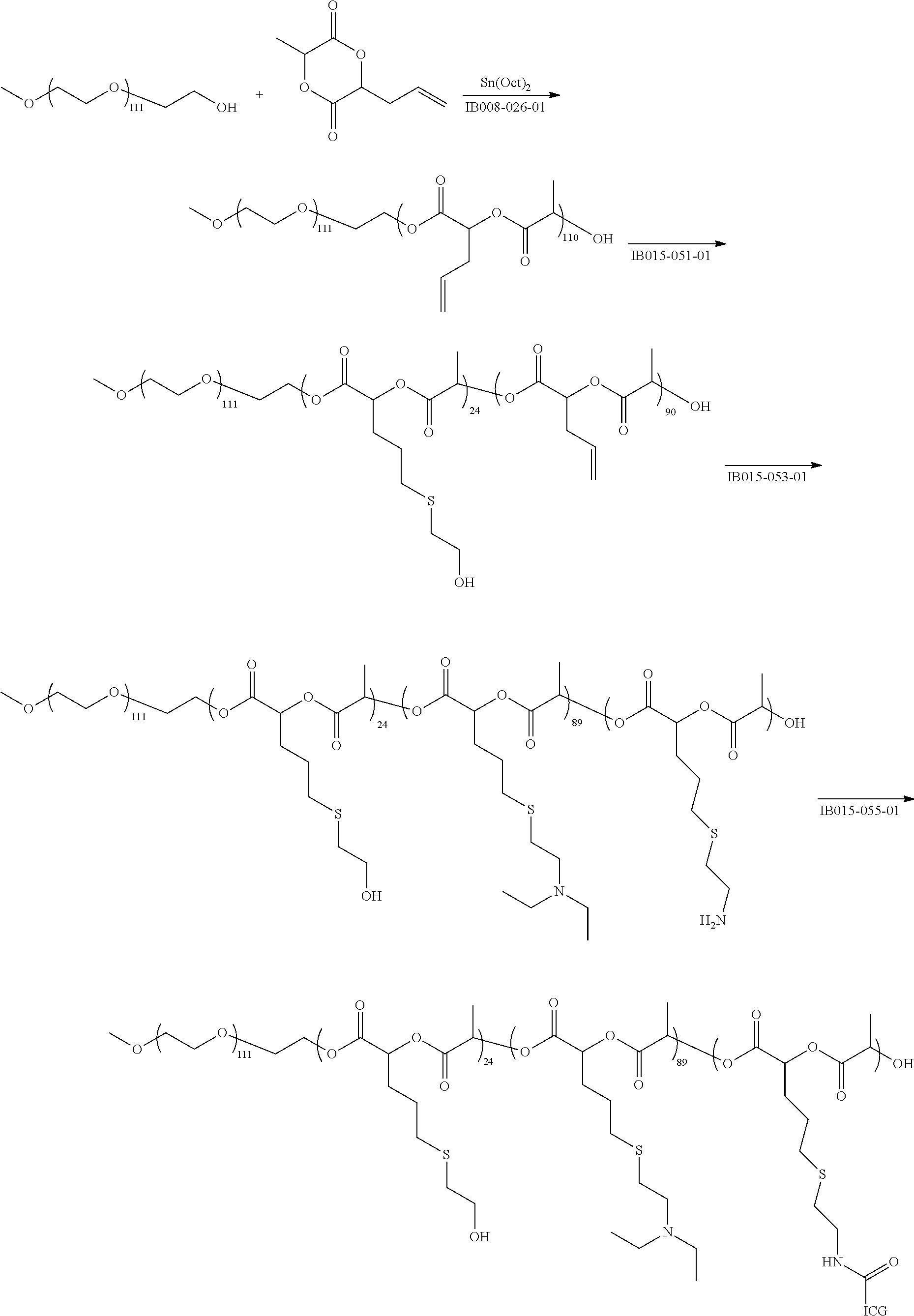

### 11.1.2 Synthesis of PLA110 (IB008-026-01)

The synthesis and purification of Example 11.1.2 was carried out according to the procedure in Example 1.2 above (using 1.02 g of 3-allyl-6-methyl-[1,4]dioxane-2,5-dione, 6 mmol) to give a total of 1.16 g of white solid polymer in 91.3% yield. ¹H NMR (400 MHz, Chloroform-d) δ 5.85-5.67 (m, 109H), 5.28-5.08 (m, 452H), 3.63 (s, 448H), 2.67 (m, 243H), 1.55 (m, 343H).

### 11.1.3 Synthesis of PLA110-C2OH (IB015-051-01)

The synthesis and purification of Example 11.1.3 was carried out according to the procedure in Example 2 above (using water as the solvent) to give a total of 55 mg of white solid polymer in 85% yield. ¹H NMR (400 MHz, Chloroform-d) δ 5.85-5.67 (m, 89H), 5.28-5.08 (m, 432H), 3.63 (d, J=1.2 Hz, 448H), 2.75-2.56 (m, 265H), 2.35-2.31 (m, 41H), 1.90-1.78 (m, 40H), 1.61-1.55 (m, 363H).

### 11.1.4 Synthesis of PLA110-C20H-TEE (IB015-053-01)

Synthesis and purification of Example 11.1.4 was carried out according to the procedure in Example 3 above (using dichloromethane as the solvent) to give a total of 60 mg of white solid polymer in 80% yield. ¹H NMR (400 MHz, Chloroform-d) δ 5.14 (s, 331H), 3.63 (d, J=1.2 Hz, 448H), 3.09-2.58 (m, 1251H), 2.35-2.31 (m, 45H), 1.90-1.78 (m, 238H), 1.55 (m, 473H).

### 11.1.5 Synthesis of PLA110-C20H-TEE-ICG (IB015-055-01)

The synthesis and purification of Example 11.1.5 was carried out according to the procedure in Example 4 above (using dichloromethane as the solvent) to give a total of 35 mg of green solid polymer in 79% yield. ¹H NMR (400 MHz, Chloroform-d) δ 8.08-7.28 (m, 25 H), 5.14 (s, 331H), 3.63 (d, J=1.2 Hz, 448H), 3.09-2.58 (m, 1251H), 2.35-2.31 (m, 45H), 1.90-1.78 (m, 238H), 1.55 (m, 473H).

### 11.2 PLA110-C2OH-TEE-ICG

### 11.2.1 Synthetic Route of PLA110-C2OH-TEE-ICG

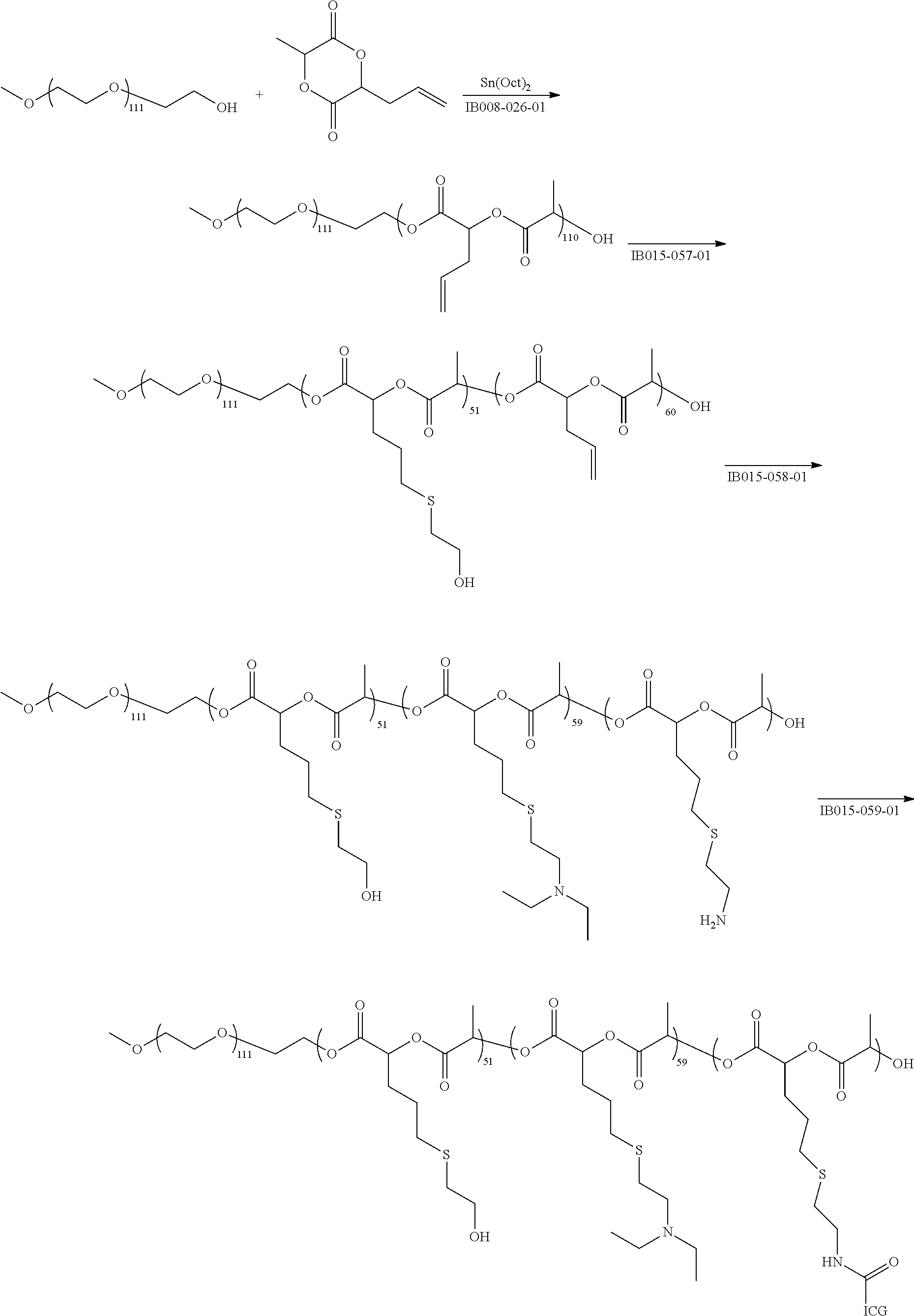

### 11.2.2 Synthesis of PLA₁₁₀ (IB008-026-01)

The synthesis and purification of Example 11.2.2 was carried out according to the procedure in Example 1.2 above (using 1.02 g of 3-allyl-6-methyl-[1,4]dioxane-2,5-dione, 6 mmol) to give a total of 1.16 g of white solid polymer in 91.3% yield. ¹H NMR (400 MHz, Chloroform-d) δ 5.85-5.67 (m, 109H), 5.28-5.08 (m, 452H), 3.63 (s, 448H), 2.67 (m, 243H), 1.55 (m, 343H).

### 11.2.3 Synthesis of PLA110-C2OH (IB015-057-01)

The synthesis and purification of Example 11.2.3 was carried out according to the procedure in Example 2 above (using water as the solvent) to give a total of 52 mg of white solid polymer in 85% yield. ¹H NMR (400 MHz, Chloroform-d) δ 5.85-5.67 (m, 59H), 5.28-5.08 (m, 397H), 3.63 (d, J=1.2 Hz, 448H), 2.75-2.56 (m, 238H), 2.37-2.32 (m, 101H), 1.90-1.78 (m, 99H), 1.61-1.55 (m, 397H).

### 11.2.4 Synthesis of PLA110-C20H-TEE (IB015-058-01)

Synthesis and purification of Example 11.2.4 was carried out according to the procedure in Example 3 above (using dichloromethane as the solvent) to give a total of 54 mg of white solid polymer in 80% yield. ¹H NMR (400 MHz, Chloroform-d) δ 5.14 (s, 337H), 3.63 (d, J=1.2 Hz, 448H), 3.09-2.58 (m, 938H), 2.37-2.32 (m, 105H), 1.90-1.78 (m, 241H), 1.55 (m, 468H).

### 11.2.5 Synthesis of PLA110-C2OH-TEE-ICG (IB015-059-01)

The synthesis and purification of Example 11.2.5 was carried out according to the procedure in Example 4 above (using dichloromethane as the solvent) to give a total of 32 mg of green solid polymer in 74% yield. ¹H NMR (400 MHz, Chloroform-d) δ 8.04-7.28(m, 28H), 5.14 (s, 337H), 3.63 (d, J=1.2 Hz, 448H), 3.09-2.58 (m, 938H), 2.37-2.32 (m, 105H), 1.90-1.78 (m, 241H), 1.55 (m, 468H).

### EXAMPLE 12

### Example Series Containing Aminoethyl Group

### 12.1 PLA110-C2NH₂-TEE-ICG

### 12.1.1 Synthetic Route of PLA110-C2NH₂-TEE-ICG

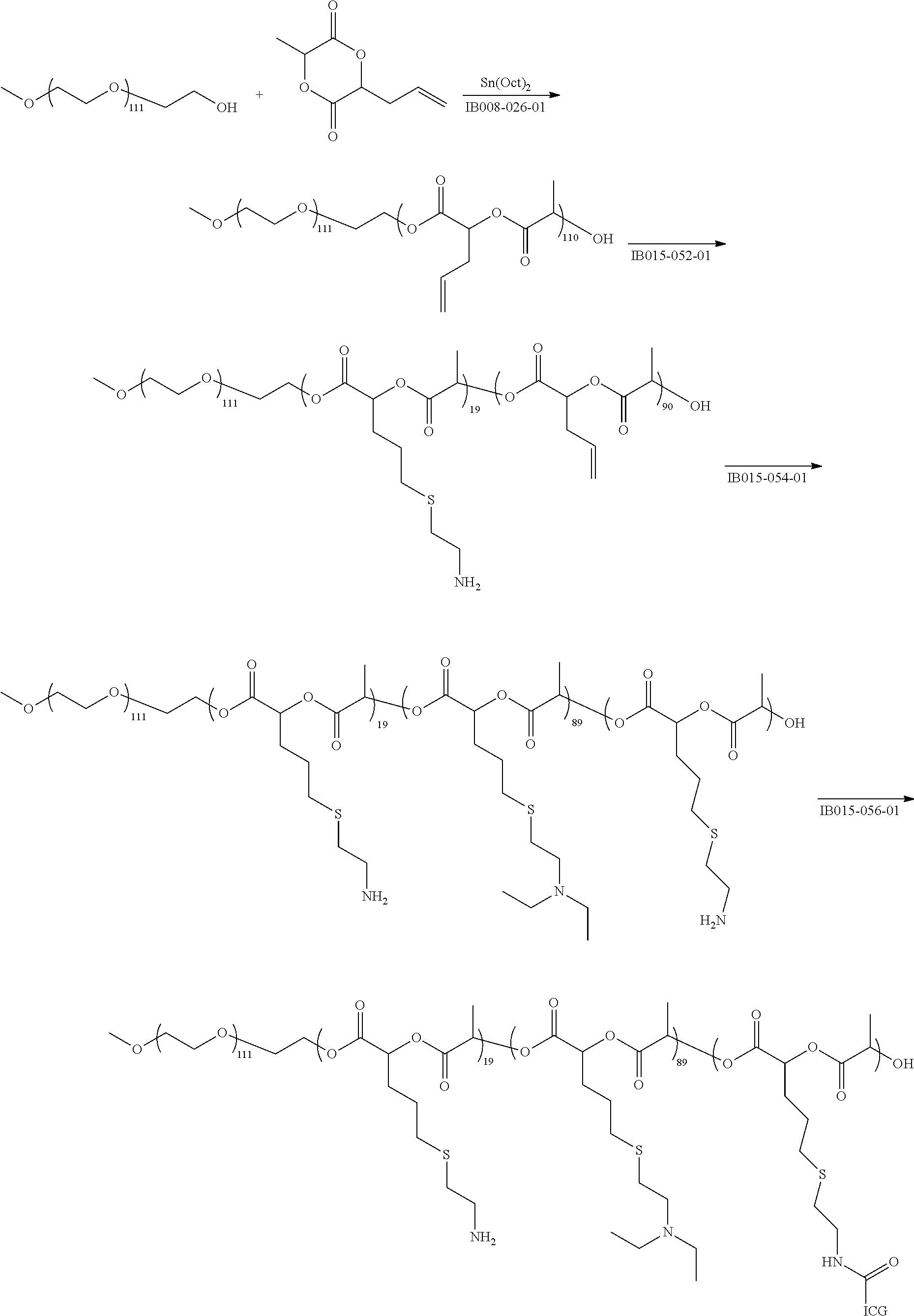

### 12.1.2 Synthesis of PLA110 (IB008-026-01)

The synthesis and purification of Example 12.1.2 was carried out according to the procedure in Example 1.2 above (using 1.02 g of 3-allyl-6-methyl-[1,4]dioxane-2,5-dione, 6 mmol) to give a total of 1.16 g of white solid polymer in 91.3% yield. ¹H NMR (400 MHz, Chloroform-d) δ 5.85-5.67 (m, 109H), 5.28-5.08 (m, 452H), 3.63 (s, 448H), 2.67 (m, 240H), 1.55 (m, 343H).

### 12.1.3 Synthesis of PLA110-C2NH₂ (IB015-052-01)

The synthesis and purification of Example 12.1.3 was carried out according to the procedure in Example 2 above (using water as the solvent) to give a total of 56 mg of white solid polymer in 85% yield. ¹H NMR (400 MHz, Chloroform-d) δ 5.85-5.67 (m, 89H), 5.28 -5.08 (m, 432H), 3.63 (d, J=1.2 Hz, 448H), 3.06-0.99 (m, 50H), 2.75-2.56 (m, 270H), 1.90-1.78 (m, 40H), 1.61-1.55 (m, 363H).

### 12.1.4 Synthesis of PLA110-C2NH₂-TEE (IB015-054-01)

Synthesis and purification of Example 12.1.4 was carried out according to the procedure in Example 3 above (using dichloromethane as the solvent) to give a total of 56 mg of white solid polymer in 80% yield. ¹H NMR (400 MHz, Chloroform-d) δ 5.14 (s, 318H), 3.63 (d, J=1.2 Hz, 448H), 3.09-2.58 (m, 1268H), 1.90-1.78 (m, 237H), 1.55 (m, 462H).

### 12.1.5 Synthesis of PLA110-C2NH₂-TEE-ICG (IB015-056-01)

The synthesis and purification of Example 12.1.5 was carried out according to the procedure in Example 4 above (using dichloromethane as the solvent) to give a total of 30 mg of green solid polymer in 75% yield. ¹H NMR (400 MHz, Chloroform-d) δ 8.07-7.29 (m, 26 H), 5.14 (s, 318H), 3.63 (d, J=1.2 Hz, 448H), 3.09-2.58 (m, 1270H), 1.90-1.78 (m, 238H), 1.55 (m, 462H).

### EXAMPLE 13

### Example Series Containing Zwitterionic Groups

### 13.1 PLA110-EMAA-TEE-ICG

### 13.1.1 Synthetic Route of PLA110-EMAA-TEE-ICG

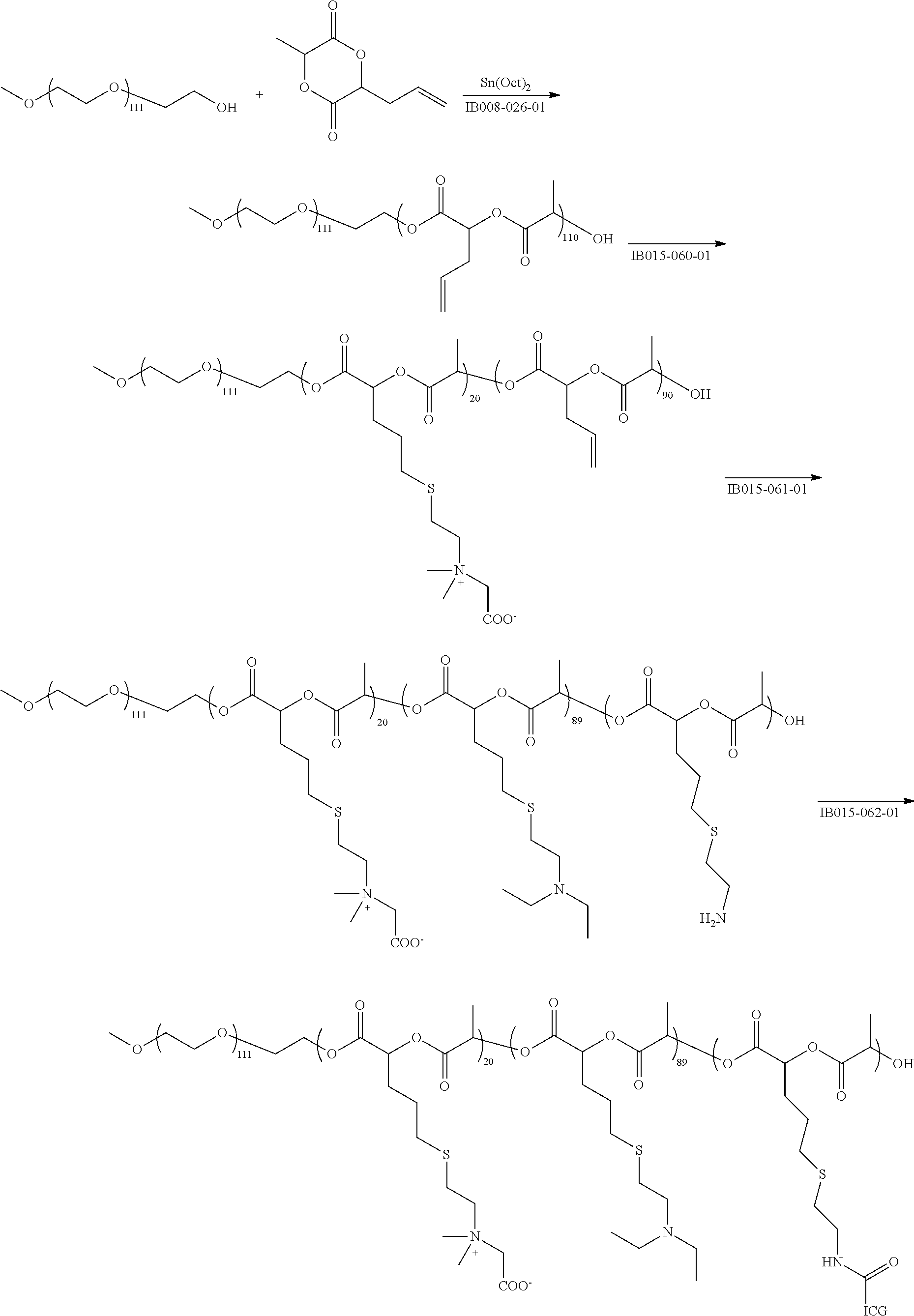

### 13.1.2 Synthesis of PLA₁₁₀ (IB008-026-01)

The synthesis and purification of Example 13.1.2 was carried out according to the procedure in Example 1.2 above (using 1.02 g of 3-allyl-6-methyl-[1,4]dioxane-2,5-dione, 6 mmol) to give a total of 1.16 g of white solid polymer in 91.3% yield. ¹H NMR (400 MHz, Chloroform-d) δ 5.85-5.67 (m, 109H), 5.28-5.08 (m, 452H), 3.63 (s, 448H), 2.67 (m, 240H), 1.55 (m, 343H).

### 13.1.3 Synthesis of PLA₁₁₀-EMAA (IB008-060-01)

The synthesis and purification of Example 13.1.3 was carried out according to the procedure in Example 2 above (using water as the solvent) to give a total of 52 mg of white solid polymer in 90% yield. ¹H NMR (400 MHz, Chloroform-d) δ 5.85-5.67 (m, 88H), 5.28-5.08 (m, 433H), 4.28 (s, 41H), 3.81-3.77 (m, 42H), 3.32 (S, 123H), 3.63 (d, J=1.2 Hz, 448H), 2.96-2.92 (m, 42H), 2.75-2.56 (m, 222H), 1.90-1.78 (m, 41H), 1.61-1.55 (m, 368H).

### 13.1.4 Synthesis of PLA110-EMAA-TEE (IB008-061-01)

The synthesis and purification of Example 13.1.4 was carried out according to the procedure in Example 3 above (using water as the solvent) to give a total of 65 mg of white solid polymer in 85% yield. ¹H NMR (400 MHz, Chloroform-d) δ 5.14 (s, 335H), 4.28 (s, 39H), 3.81-3.77 (m, 43H), 3.63 (d, J=1.2 Hz, 448H), 3.32 (S, 126H), 3.09-2.58 (m, 1233H), 1.90-1.78 (m, 243H), 1.55 (m, 468H).

### 13.1.5 Synthesis of PLA110-EMAA-TEE-ICG (IB008-062-01)

The synthesis and purification of Example 13.1.5 was carried out according to the procedure in Example 4 above (using dichloromethane as the solvent) to give a total of 32 mg of green solid polymer in 74% yield. ¹H NMR (400 MHz, Chloroform-d) δ 8.21-7.30 (m, 27H) 5.14 (s, 334H), 4.28 (s, 39H), 3.81-3.77 (m, 38H), 3.63 (d, J=1.2 Hz, 448H), 3.32 (S, 117H), 3.09-2.58 (m, 1225H), 1.90-1.78 (m, 241H), 1.55 (m, 465H).

### EXAMPLE 14

### 14.1 In Vivo Imaging Experiments of Subcutaneous Breast Cancer Tumors in Mice After Tail-Vein Injection of a Polymer Nano-Particle Imaging Probe Containing C₂H₄OH Hydrophilic Groups

Animal Model: Female Balb/c nude mice (4-6 weeks age) were inoculated with 4T1 cells (app. 2×10⁶/mouse) into the right flank. Imaging Experiment is conducted on mice when tumor volume reaches 200-400 mm³.

Dose of imaging agents: nano-particlesolution of imaging probe IB015-055-01 (Example 11.1.5) was injected via tail vein at 2.5 mg/kg.

In vivo fluorescence imaging: A fluorescence imaging system (PerkinElmer, IVIS spectrum CT, Made: U.S.A, each fluorescence imaging capture uses the system-default ICG filters with identical imaging parameters) is used to observe the in vivo biodistribution and tumor accumulation at different time points. As exhibited in FIG. 6, intense fluorescence is observed in tumor 24 hours post injection. After in vivo fluorescence imaging, mice were executed and major organs were collected for fluorescence intensity quantification to characterize tissue distribution of the imaging agent.

### 14.2 Measurement of the Ratio of Tumor to Healthy Tissue (Muscle) After Administration of Imaging Agent (IB015-055-01, Whose Structure Corresponds to Example 11.1.5)

After administration, after 24 hours of in vivo fluorescence imaging, mice were executed to collect samples of tumors, muscles, and major organs (heart, liver, spleen, lungs, and kidneys), which were then subjected to fluorescence imaging. After imaging, the total and mean fluorescence intensities were determined by using the same size of the region of interest (ROI) and delineating the area of the different tissues (typical sampling imaging results and values can be seen in FIG. 6). The TNR (Tumor/Normal Tissue Ratio) was then calculated using the “Total Fluorescence Intensity of Tumor ROI”/“Total Fluorescence Intensity of Muscle ROI”.

The results obtained are shown in FIG. 7 (Table), which shows that the fluorescence intensity of the tumor increases rapidly from 0 to 24 hours after the administration of the drug, and the ratio of tumor/muscle (TNR) also reaches up to 21.1-fold within 24 hours after the administration of the drug. Based on the values in the Table, the calculated ratio of lymph nodes (with strong fluorescence) to muscle can be about 13.3.

### EXAMPLE 15

### 15.1 In Vivo Imaging Experiments of Subcutaneous Breast Cancer Tumors in Mice After Tail-Vein Injection of a Polymer Nano-Particle Imaging Probe Containing C₉H₁₉ Hydrophobic Groups

Animal Model: Female Balb/c nude mice (4-6 weeks age) were inoculated with 4T1 cells (app. 2×10⁶/mouse) into the right flank. Imaging Experiment is conducted on mice when tumor volume reaches 200-400 mm³.

Dose of imaging agents: nano-particlesolution of imaging probe IB015-038-01 (Example 6.1.5) was injected via tail vein at 2.5mg/kg.

In vivo fluorescence imaging: A fluorescence imaging system (PerkinElmer, IVIS spectrum CT, Made: U.S.A, each fluorescence imaging capture uses the system-default ICG filters with identical imaging parameters) is used to observe the in vivo biodistribution and tumor accumulation at different time points. As exhibited in FIG. 8, intense fluorescence is observed in tumor 24 hours post injection. After in vivo fluorescence imaging, mice were executed and major organs were collected for fluorescence intensity quantification to characterize tissue distribution of the imaging agent.

### 16.2 Determination of the Ratio of Tumor to Healthy Tissue (Muscle) After Administration of Imaging Agent (IB015-038-01, Whose Structure Corresponds to Example 6.1.5)

After administration, after 24 hours of in vivo fluorescence imaging, mice were executed to collect samples of tumors, muscles, and major organs (heart, liver, spleen, lungs, and kidneys), which were then subjected to fluorescence imaging. After imaging, the total and mean fluorescence intensities were determined by using the same size of the region of interest (ROI) and delineating the area of the different tissues (typical sampling imaging results and values can be seen in FIG. 8). The TNR (Tumor/Normal Tissue Ratio) was then calculated using the “Total Fluorescence Intensity of Tumor ROI”/“Total Fluorescence Intensity of Muscle ROI”.

The results obtained are shown in FIG. 9 (Table), which shows that the fluorescence intensity of the tumor increases rapidly from 0 to 24 hours after the administration of the drug, and the ratio of tumor/muscle (TNR) also reaches up to 13.0-fold within 24 hours after the administration of the drug. Based on the values in the Table, the calculated ratio of lymph nodes (with strong fluorescence) to muscle can be about 4.9.

### EXAMPLE 16

### 16.1 In Vivo Imaging Experiments of Subcutaneous Breast Cancer Tumors in Mice After Tail-Vein Injection of a Polymer Nano-Particle Imaging Probe Containing Cholesterol Hydrophobic Groups

Animal Model: Female Balb/c nude mice (4-6 weeks age) were inoculated with 4T1 cells (app. 2×10 6/mouse) into the right flank. Imaging Experiment is conducted on mice when tumor volume reaches 200-400 mm³.

Dose of imaging agents: nano-particlesolution of imaging probe IB015-050-01 (Example 10.1.5) was injected via tail vein at 2.5 mg/kg.

In vivo fluorescence imaging: A fluorescence imaging system (PerkinElmer, IVIS spectrum CT, Made: U.S.A, each fluorescence imaging capture uses the system-default ICG filters with identical imaging parameters) is used to observe the in vivo biodistribution and tumor accumulation at different time points. Intense fluorescence is observed in tumor 24 hours post injection. After in vivo fluorescence imaging, mice were executed and major organs were collected for fluorescence intensity quantification to characterize tissue distribution of the imaging agent. The result is shown in FIG. 10.

### 16.2 Measurement of the Ratio of Tumor to Healthy Tissue (Muscle) After Administration of Imaging Agent (IB015-050-01, Whose Structure Corresponds to Example 10.1.5)

24 hours post administration, mice were executed to collect samples of tumors, muscles, and major organs (heart, liver, spleen, lungs, and kidneys), which were then subjected to fluorescence imaging. After imaging, the total and mean fluorescence intensities were determined by using the same size of the region of interest (ROI) and delineating the area of the different tissues (typical sampling imaging results and values can be seen in FIG. 10). The TNR (Tumor/Normal Tissue Ratio) was then calculated using the “Total Fluorescence Intensity of Tumor ROI”/“Total Fluorescence Intensity of Muscle ROI”.

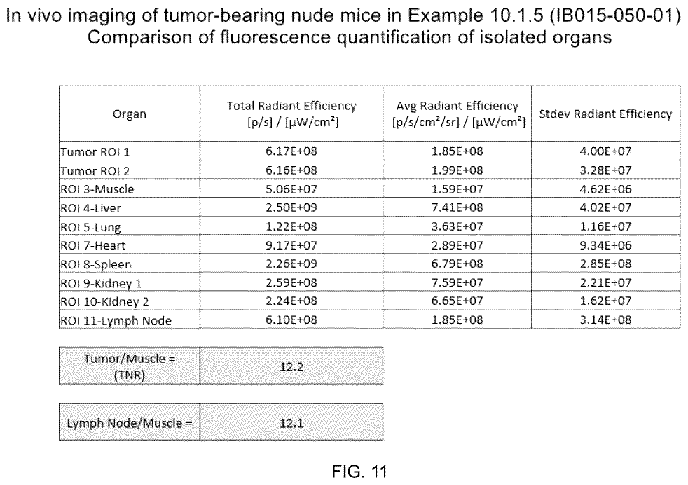

The results obtained are shown in FIG. 11 (Table), which shows that the fluorescence intensity of the tumor increases rapidly from 0 to 24 hours after the administration of the drug, and the ratio of tumor/muscle (TNR) also reaches up to 12.2-fold within 24 hours after the administration of the drug. Based on the values in the Table, the calculated ratio of lymph nodes (with strong fluorescence) to muscle can be about 12.1.

As mentioned above, the present disclosure effectively overcomes various shortcomings in the traditional technology and has high industrial utilization value.

The above-mentioned embodiments are merely illustrative of the principle and effects of the present disclosure instead of limiting the present disclosure. Modifications or variations of the above-described embodiments may be made by those skilled in the art without departing from the spirit and scope of the present disclosure. Therefore, all equivalent modifications or changes made by those who have common knowledge in the art without departing from the spirit and technical concept disclosed by the present disclosure shall be still covered by the claims of the present disclosure.

1. A functionalized diblock copolymer, wherein the chemical structure of the functionalized diblock copolymer is shown in Formula III: 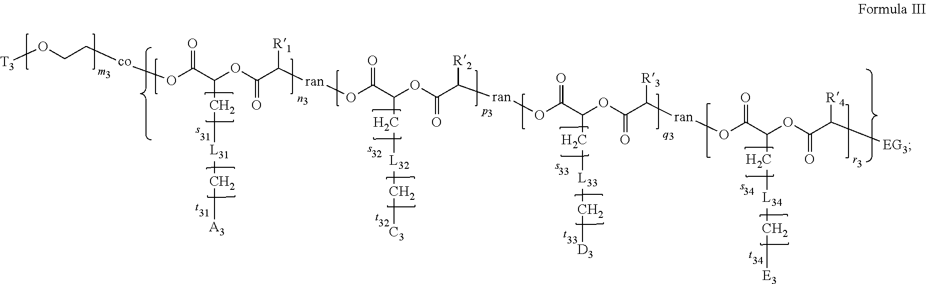
wherein in Formula III, m₃=22˜1136, n₃=10˜500, p₃=0.5˜50, q₃=0˜500, r₃=0˜200;
s₃₁=1˜10, s₃₂=1˜10, s₃₃=1˜10, s₃₄=1˜10;
t₃₁=1˜10, t₃₂=1˜10, t₃₃=1˜10, t₃₄=1˜10;
L₃₁, L₃₂, L₃₃ and L₃₄ are linking groups;
R′₁, R′₂, R′₃ and R′₄ are independently selected from H, C1-C20 alkyl and C3-C10 cycloalkyl;
A₃ is selected from protonatable groups;
C₃ is selected from fluorescent molecular groups;
D₃ is selected from delivery molecular groups;
E₃ is selected from hydrophilic/hydrophobic groups;
T₃ is selected from capping groups; and
EG₃ is selected from capping groups.

2. The functionalized diblock copolymer of claim 1, wherein in Formula III, s₃₁=1˜5, s₃₂=1˜5, s₃₃=1˜5, s₃₄=1˜5;
t₃₁=1˜6, t₃₂=1˜6, t₃₃=1˜6, t₃₄=1˜6;
a molecular weight of the polyethylene glycol block is in a range of 1000˜50000 Da, and a molecular weight of the polylactide block is in a range of 1000˜130000 Da;
and/or, a critical micelle concentration (CMC) of the functionalized diblock copolymer is less than 50 μg/mL.

3. The functionalized diblock copolymer of claim 1, wherein in Formula III, s₃₁=1˜5, s₃₂=1˜5, s₃₃=1˜5, s₃₄=1˜5;
L₃₁, L₃₂, L₃₃, L₃₄ are independently selected from —S—, —O—, —OC(O)—, —C(O)O—, SC(O)—, —C(O)—, —OC(S)—, —C(S)O—, —SS—,—C(R₁)═N—, —N═C(R₂)—, —C(R₃)═N—O—, —O—N═C(R₄)—, —N(R₅)C(O)—, —C(O)N(R₆)—, —N(R₇)C(S)—, —C(S)N(R₈)—, —N(R₉)C(O)N(R₁₀)—, —OS(O)O—, —OP(O)O—, —OP(O)N—, —NP(O)O—, and —NP(O)N—, and wherein R₁˜R₁₀ are each independently selected from H, C1-C10 alkyl, and C3-C10 cycloalkyl.
A₃ is 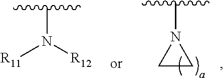
wherein R₁₁ and R₁₂ are independently selected from C1-C10 alkyl, C2-C10 alkenyl, C2-C10 alkynyl, C3-C10 cycloalkyl, aryl and heteroaryl; a=1-10, and a is a positive integer; preferably, R₁₁ is ethyl and R₁₂ is ethyl, or, R₁₁ is ethyl and R₁₂ is n-propyl, or, R₁₁ is n-propyl and R₁₂ is n-propyl, or, R₁₁ is n-propyl and R₁₂ is n-butyl, or, R₁₁ is n-butyl and R₁₂ is n-butyl;
C₃ comprises one or more of ICG (indocyanine green), METHYLENE BLUE (methylene blue), CY3, CY3.5, CY5, CY5.5, CY7, CY7.5, BDY630, BDY650, BDY-TMR, Tracy 645, and Tracy 652;
D₃ is selected from fluorescence quenching group and drug molecule group, wherein the fluorescence quenching group is preferably selected from BHQ-0, BHQ-1, BHQ-2, BHQ-3, BHQ-10, QXL-670, QXL-610, QXL-570, QXL_{520,} QXL-490, QSY35, QSY7, QSY21, QXL_{680,} Iowa Black RQ and Iowa Black FQ; wherein the drug molecule is preferably selected from chemotherapeutic drugs, more preferably selected from 5-ALA (5-Aminolevulinic acid), nucleic acid drugs, paclitaxel, cisplatin, doxorubicin, irinotecan and SN38;
E₃ is a hydrophilic/hydrophobic group, and is selected from H, C1-C18 alkyl, —O—R₁₁, —S—R₁₂, wherein R₁₁ ˜R₁₂ are each independently selected from H, C1-C18 alkyl, C3-C10 cycloalkyl, aryl, and heteroaryl; preferably, E₃ is selected from —(CH₂—CH₂—O)n-H (n=1˜30), —(R₁₄)—NH₂ where R₁₄ is repeated methylene group (—CH₂-)n (n=1˜18), —(R₁₅)—OH where R₁₅ is repeated methylene group (—CH₂-)n (n=1˜18), sugar groups such as monosaccharides and/or disaccharides; or preferably, E₃ is selected from cholesterol and its' derivatives, hydrophobic vitamins such as Vitamin E and Vitamin D, and zwitterionic groups with exemplar structures shown below;
preferably, the above-mentioned hydrophilic or hydrophobic groups are each used alone as an E₃ group, or are used in combination as an E₃ group; wherein when hydrophilic and hydrophobic groups are used simultaneously, the total number of all the hydrophilic and hydrophobic groups is described as r₃ where r_{3,A} is the total number of hydrophilic groups and r_{3,B} is the total number of hydrophobic groups, and accordingly, (r_{3,A}+r_{3,B}) is in a range of 1-200, and r_{3,A} is less than 151;
more preferably, the chemical structural formula of the cholesterol or cholesterol derivative, vitamin D, or vitamin E is shown in one of the following: 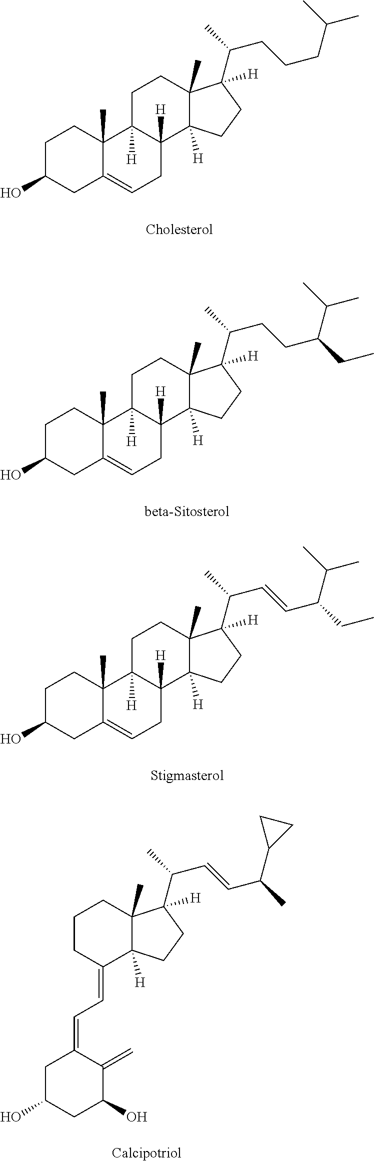 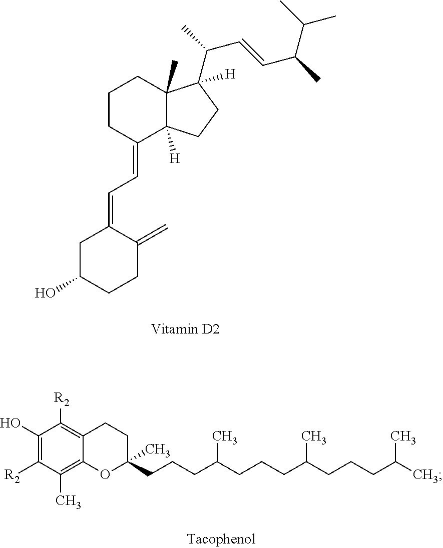
more preferably, the chemical structural formula of the zwitterionic group is shown in one of the following: 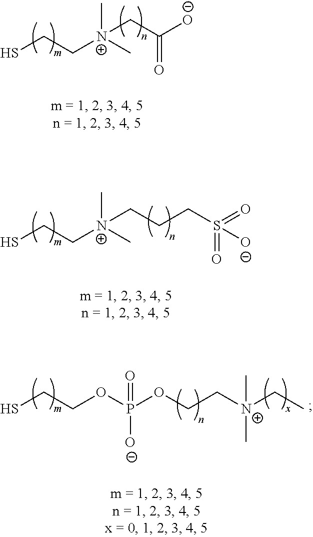
E₃ is further preferably selected from the group consisting of n-nonyl, n-octyl, n-butyl, n-propyl, ethyl, methyl, n-octadecyl, n-heptadecyl, cholesterol, cholesterol derivatives, hydroxyethyl, hydroxymethyl, hydroxypropyl, hydroxybutyl, zwitterionic group, a combination of zwitterionic group and n-nonyl, and a combination of zwitterionic group and n-octyl;
T₃ is selected from —CH₃ and —H;
EG₃ is selected from —Y—R₁₃, wherein Y is selected from O, S, and N, and R₁₃ is selected from H, C1-C20 alkyl, C3-C10 cycloalkyl, aryl and heteroaryl.

4. The functionalized diblock copolymer of claim 1, wherein in Formula III, m₃=22˜1136, n₃=10˜500, p₃=1˜50, q₃=0, r₃=0;
or, in formula III, m₃=22˜1136, n₃=10˜500, p₃=0.5˜50, q₃=0, r₃=1˜200;
or, in formula III, m₃=22˜1136, n₃=10˜500, p₃=0.5˜50, q₃=1˜500, r₃=0.

5. The functionalized diblock copolymer of claim 1, wherein the chemical structural formula of the functionalized diblock copolymer is shown as one of the following: 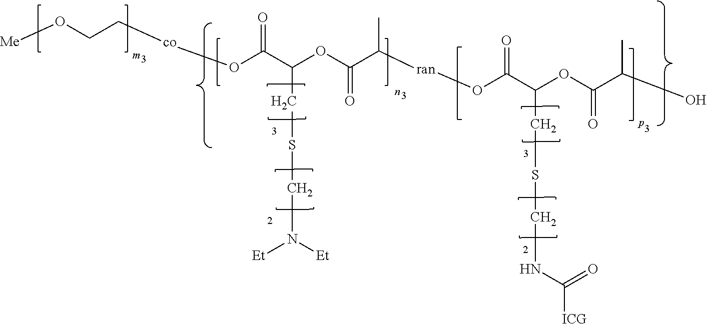
wherein m₃ is in a range of 22˜1136, preferably 44˜226, n₃ is in a range of 10˜500, preferably 30˜200, and p₃ is in a range of 0.5˜5; 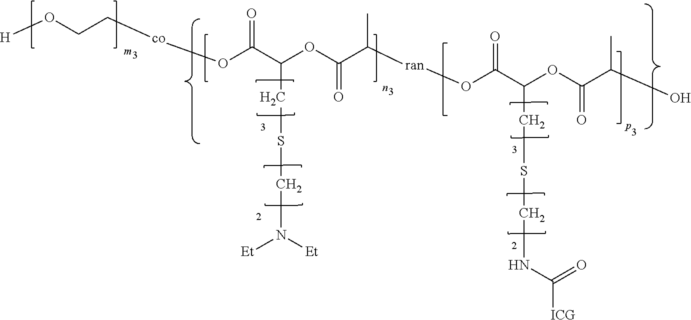
wherein m₃ is in a range of 22˜1136, preferably 44˜226, n₃ is in a range of 10˜500, preferably 30˜200, and p₃ is in a range of 0.5˜5; 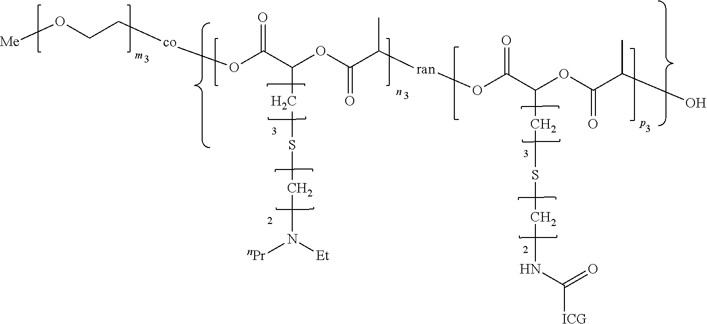
wherein m₃ is in a range of 22˜1136, preferably 44˜226, n₃ is in a range of 10˜500, preferably 30˜200, and p₃ is in a range of 0.5˜5; 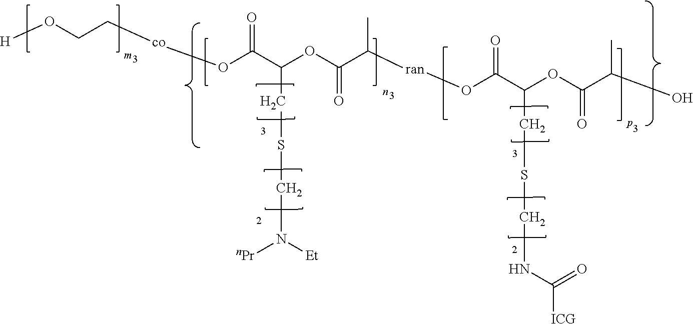
wherein m₃ is in a range of 22˜1136, preferably 44˜226, n₃ is in a range of 10˜500, preferably 30˜200, and p₃ is in a range of 0.5˜5; 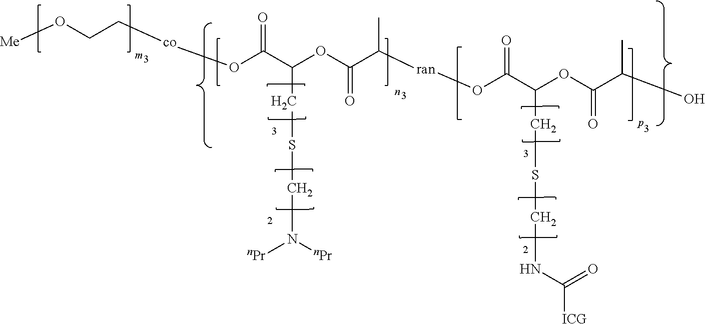
wherein m₃ is in a range of 22˜1136, preferably 44˜226, n₃ is in a range of 10˜500, preferably 30˜200, and p₃ is in a range of 0.5˜5; 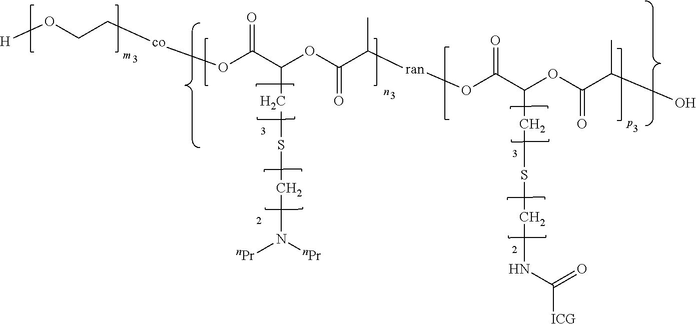
wherein m₃ is in a range of 22˜1136, preferably 44˜226, n₃ is in a range of 10˜500, preferably 30˜200, and p₃ is in a range of 0.5˜5; 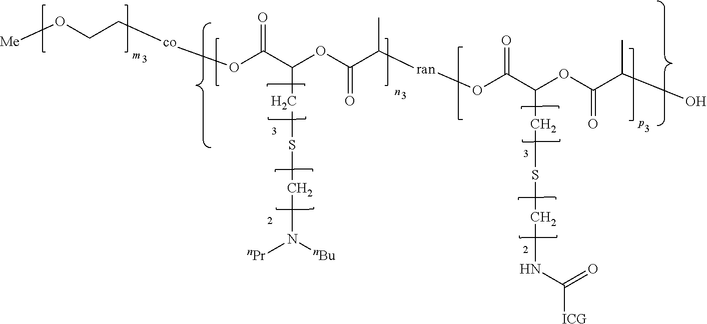
wherein m₃ is in a range of 22˜1136, preferably 44˜226, n₃ is in a range of 10˜500, preferably 30˜200, and p₃ is in a range of 0.5˜5; 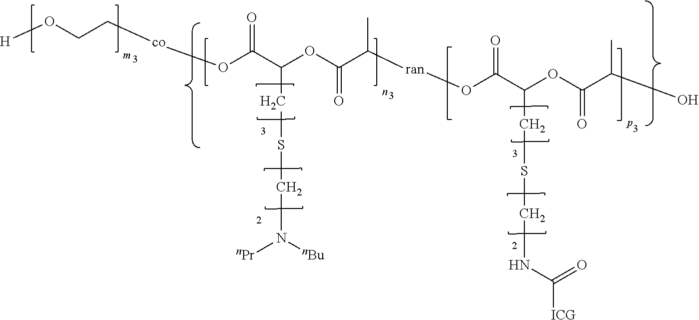
wherein m₃ is in a range of 22˜1136, preferably 44˜226, n₃ is in a range of 10˜500, preferably 30˜200, and p₃ is in a range of 0.5˜5; 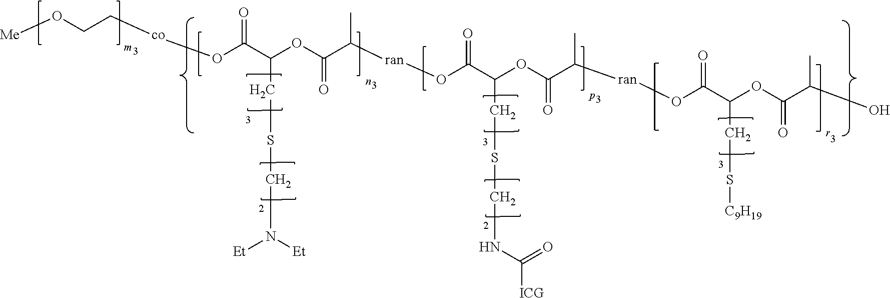
wherein m₃ is in a range of 22-1136, preferably 44-226, n₃ is in a range of 10-500, preferably 30-200, p₃ is in a range of 0.5-5, and r₃ is in a range of 1-200; 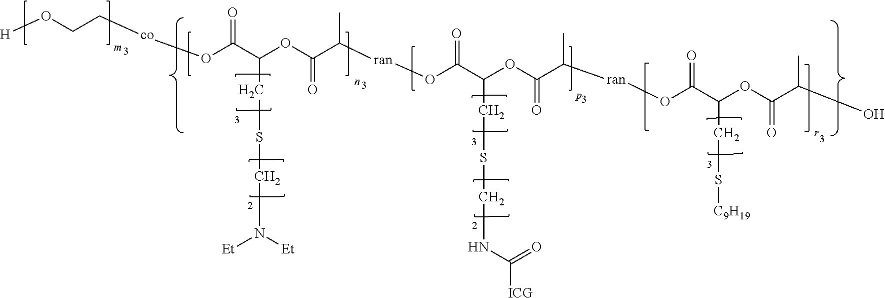
wherein m₃ is in a range of 22-1136, preferably 44-226, n₃ is in a range of 10-500, preferably 30-200, p₃ is in a range of 0.5-5, and r₃ is in a range of 1-200; 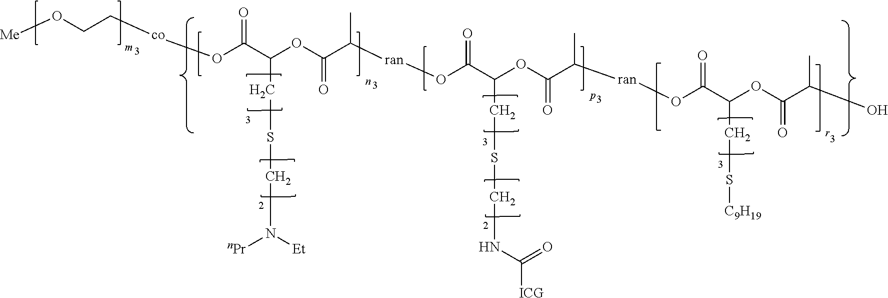
wherein m₃ is in a range of 22-1136, preferably 44-226, n₃ is in a range of 10-500, preferably 30-200, p₃ is in a range of 0.5-5, and r₃ is in a range of 1-200; 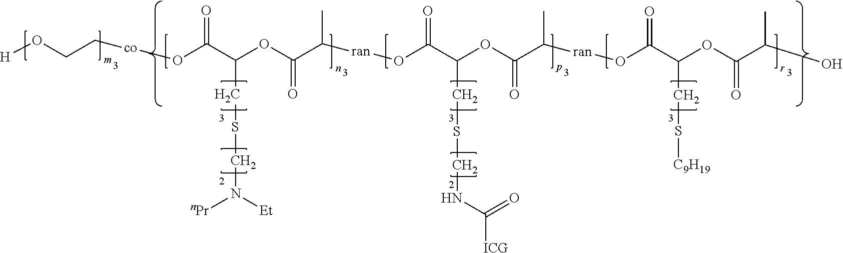
wherein m₃ is in a range of 22-1136, preferably 44-226, n₃ is in a range of 10-500, preferably 30-200, p₃ is in a range of 0.5-5, and r₃ is in a range of 1-200; 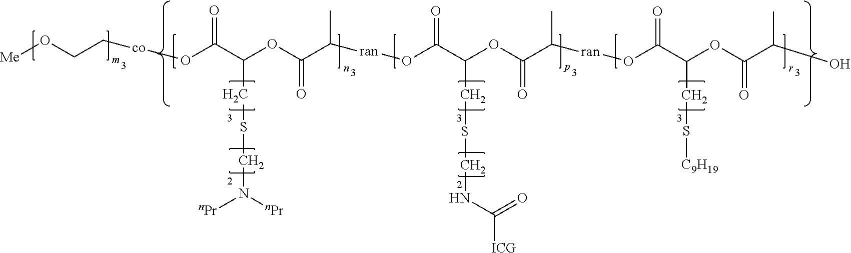
wherein m₃ is in a range of 22-1136, preferably 44-226, n₃ is in a range of 10-500, preferably 30-200, p₃ is in a range of 0.5-5, and r₃ is in a range of 1-200; 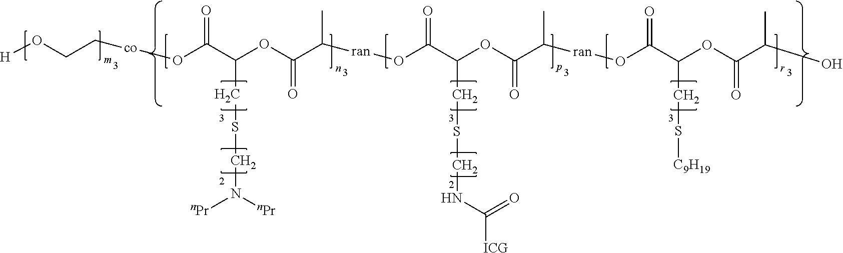
wherein m₃ is in a range of 22-1136, preferably 44-226, n₃ is in a range of 10-500, preferably 30-200, p₃ is in a range of 0.5-5, and r₃ is in a range of 1-200; 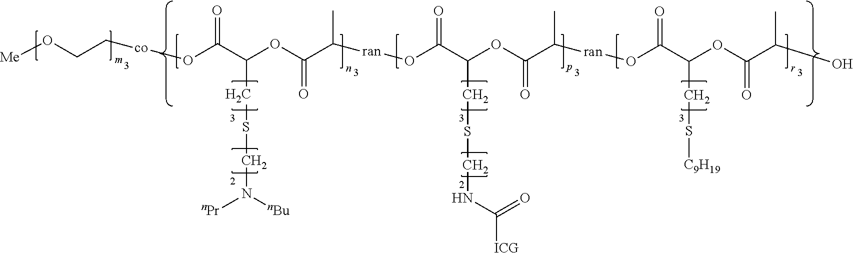
wherein m₃ is in a range of 22-1136, preferably 44-226, n₃ is in a range of 10-500, preferably 30-200, p₃ is in a range of 0.5-5, and r₃ is in a range of 1-200; 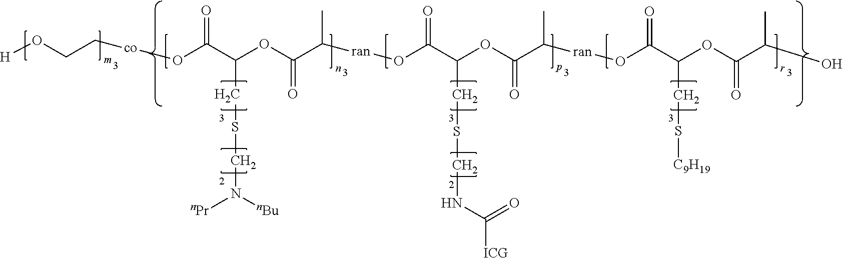
wherein m₃ is in a range of 22-1136, preferably 44-226, n₃ is in a range of 10-500, preferably 30-200, p₃ is in a range of 0.5-5, and r₃ is in a range of 1-200; 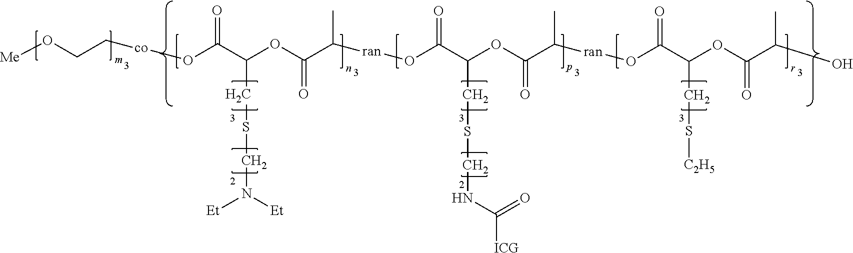
wherein m₃ is in a range of 22-1136, preferably 44-226, n₃ is in a range of 10-500, preferably 30-200, p₃ is in a range of 0.5-5, and r₃ is in a range of 1-200; 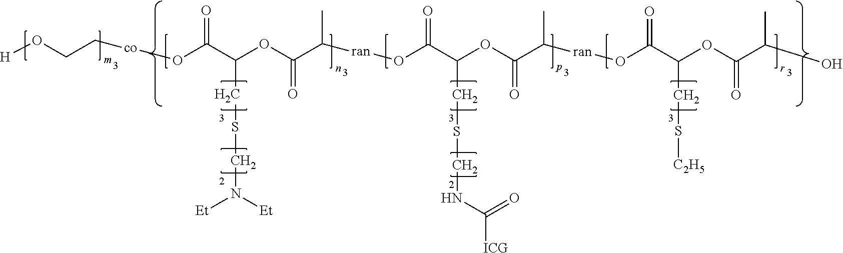
wherein m₃ is in a range of 22-1136, preferably 44-226, n₃ is in a range of 10-500, preferably 30-200, p₃ is in a range of 0.5-5, and r₃ is in a range of 1-200; 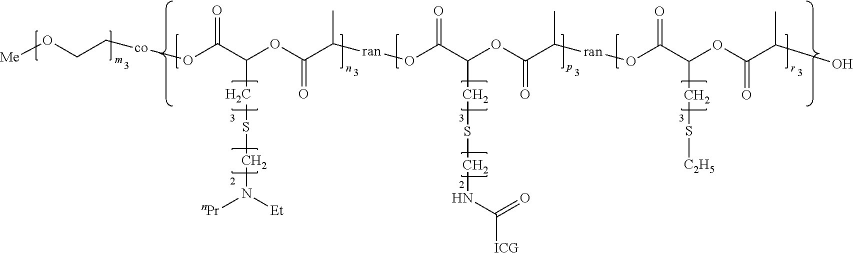
wherein m₃ is in a range of 22-1136, preferably 44-226, n₃ is in a range of 10-500, preferably 30-200, p₃ is in a range of 0.5-5, and r₃ is in a range of 1-200; 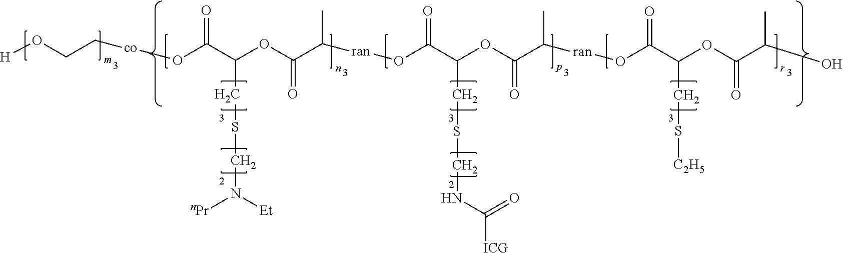
wherein m₃ is in a range of 22-1136, preferably 44-226, n₃ is in a range of 10-500, preferably 30-200, p₃ is in a range of 0.5-5, and r₃ is in a range of 1-200; 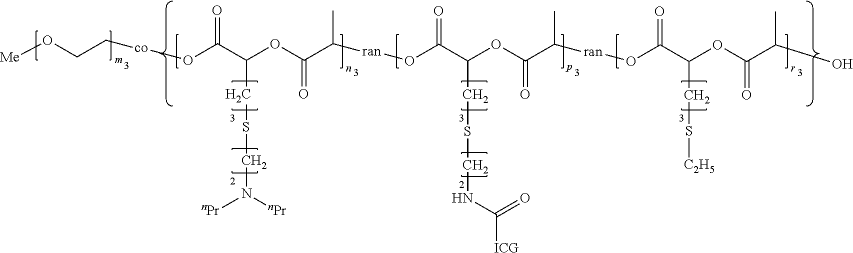
wherein m₃ is in a range of 22-1136, preferably 44-226, n₃ is in a range of 10-500, preferably 30-200, p₃ is in a range of 0.5-5, and r₃ is in a range of 1-200; 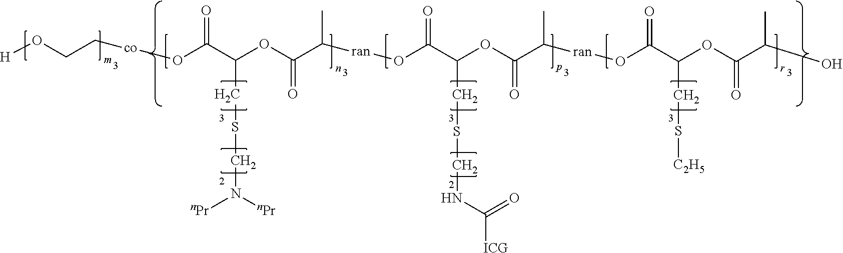
wherein m₃ is in a range of 22-1136, preferably 44-226, n₃ is in a range of 10-500, preferably 30-200, p₃ is in a range of 0.5-5, and r₃ is in a range of 1-200; 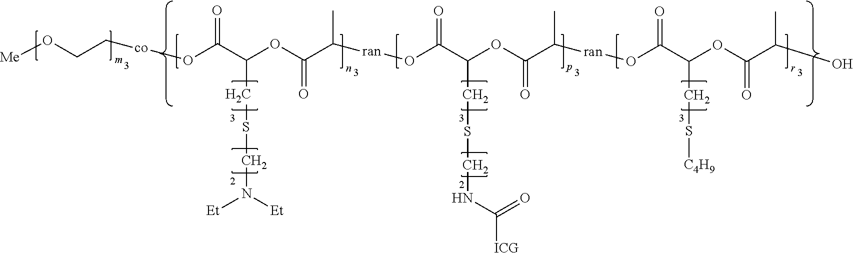
wherein m₃ is in a range of 22-1136, preferably 44-226, n₃ is in a range of 10-500, preferably 30-200, p₃ is in a range of 0.5-5, and r₃ is in a range of 1-200; 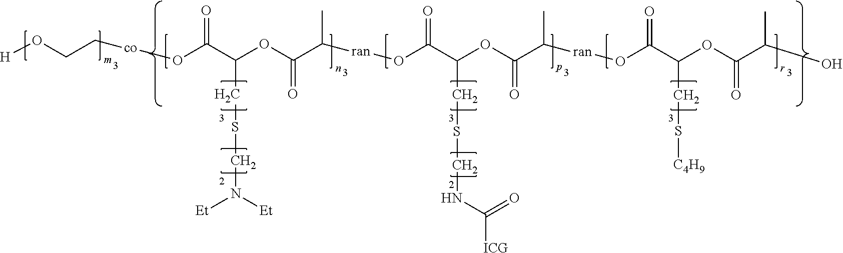
wherein m₃ is in a range of 22-1136, preferably 44-226, n₃ is in a range of 10-500, preferably 30-200, p₃ is in a range of 0.5-5, and r₃ is in a range of 1-200; 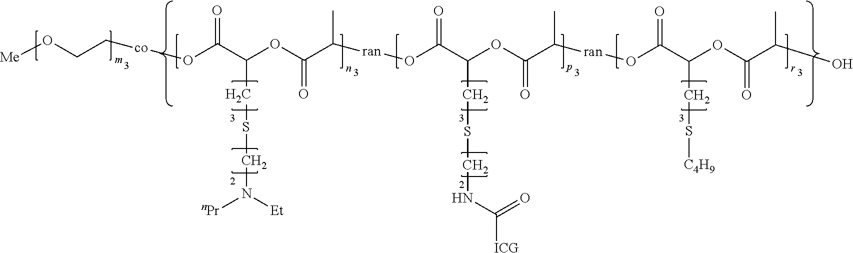
wherein m₃ is in a range of 22-1136, preferably 44-226, n₃ is in a range of 10-500, preferably 30-200, p₃ is in a range of 0.5-5, and r₃ is in a range of 1-200; 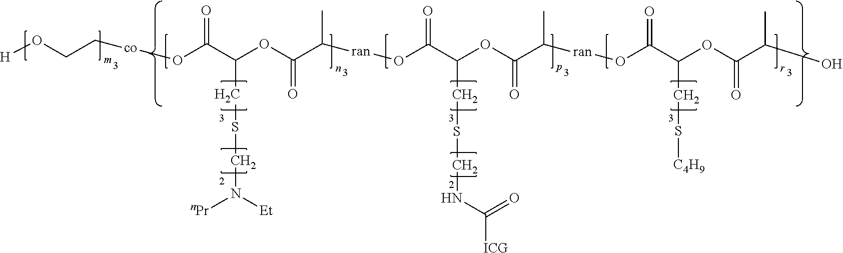
wherein m₃ is in a range of 22-1136, preferably 44-226, n₃ is in a range of 10-500, preferably 30-200, p₃ is in a range of 0.5-5, and r₃ is in a range of 1-200; 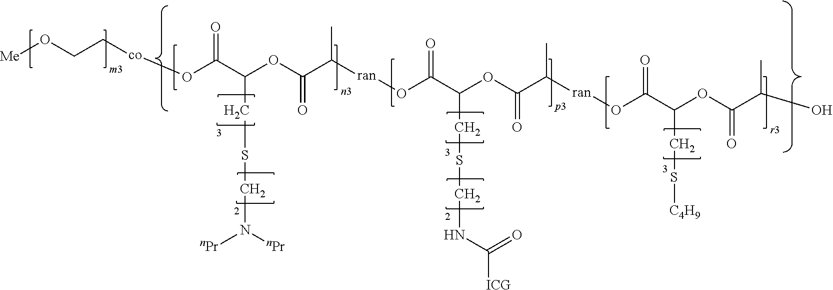
wherein m₃ is in a range of 22-1136, preferably 44-226, n₃ is in a range of 10-500, preferably 30-200, p₃ is in a range of 0.5-5, and r₃ is in a range of 1-200; 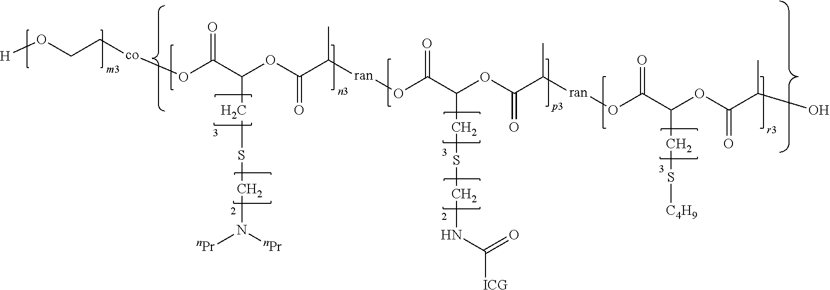
wherein m₃ is in a range of 22-1136, preferably 44-226, n₃ is in a range of 10-500, preferably 30-200, p₃ is in a range of 0.5-5, and r₃ is in a range of 1-200; 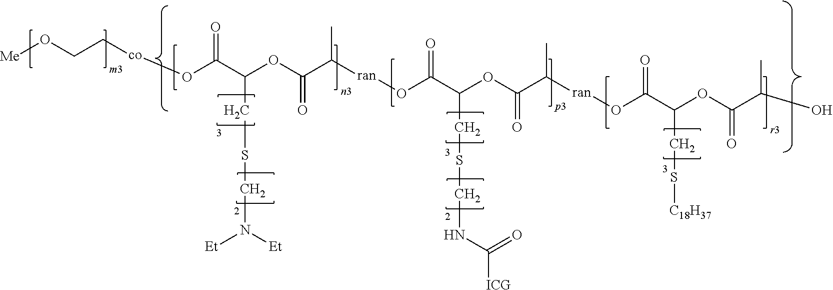
wherein m₃ is in a range of 22-1136, preferably 44-226, n₃ is in a range of 10-500, preferably 30-200, p₃ is in a range of 0.5-5, and r₃ is in a range of 1-200; 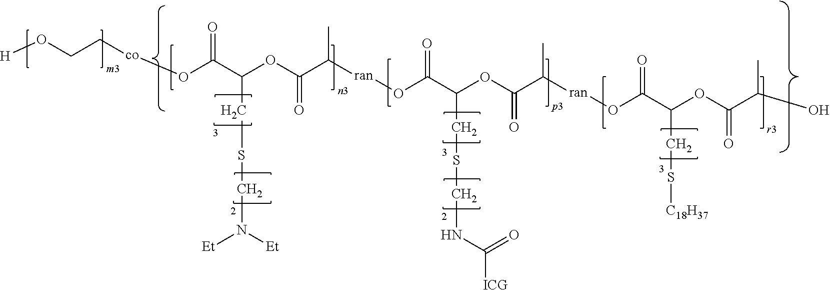
wherein m₃ is in a range of 22-1136, preferably 44-226, n₃ is in a range of 10-500, preferably 30-200, p₃ is in a range of 0.5-5, and r₃ is in a range of 1-200; 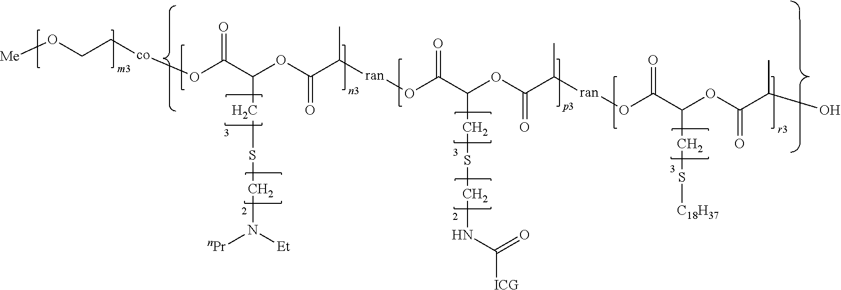
wherein m₃ is in a range of 22-1136, preferably 44-226, n₃ is in a range of 10-500, preferably 30-200, p₃ is in a range of 0.5-5, and r₃ is in a range of 1-200; 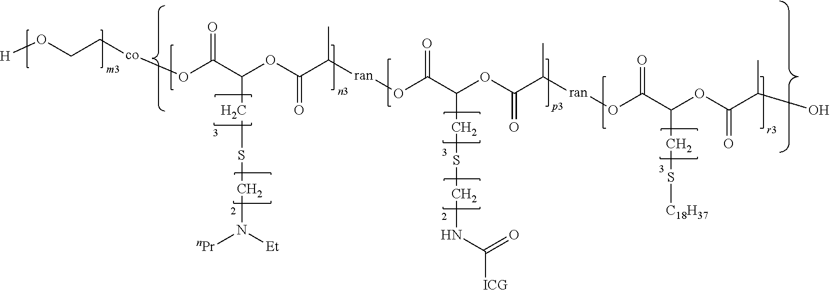
wherein m₃ is in a range of 22-1136, preferably 44-226, n₃ is in a range of 10-500, preferably 30-200, p₃ is in a range of 0.5-5, and r₃ is in a range of 1-200; 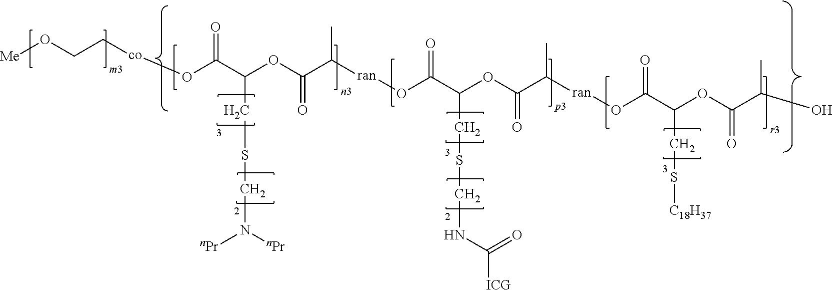
wherein m₃ is in a range of 22-1136, preferably 44-226, n₃ is in a range of 10-500, preferably 30-200, p₃ is in a range of 0.5-5, and r₃ is in a range of 1-200; 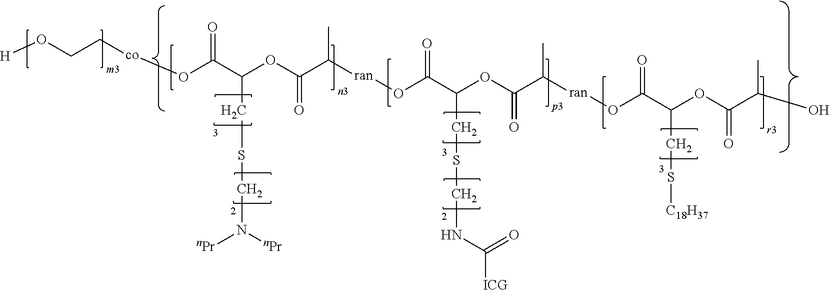
wherein m₃ is in a range of 22-1136, preferably 44-226, n₃ is in a range of 10-500, preferably 30-200, p₃ is in a range of 0.5-5, and r₃ is in a range of 1-200; 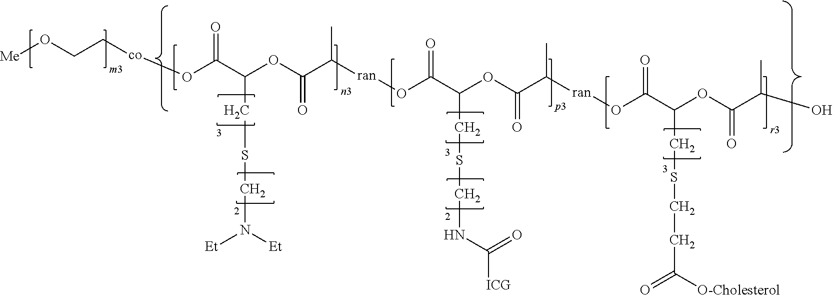
wherein m₃ is in a range of 22-1136, preferably 44-226, n₃ is in a range of 10-500, preferably 30-200, p₃ is in a range of 0.5-5, and r₃ is in a range of 1-200; 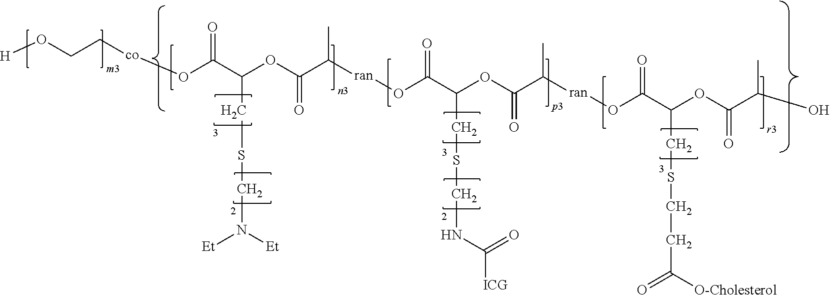
wherein m₃ is in a range of 22-1136, preferably 44-226, n₃ is in a range of 10-500, preferably 30-200, p₃ is in a range of 0.5-5, and r₃ is in a range of 1-200; 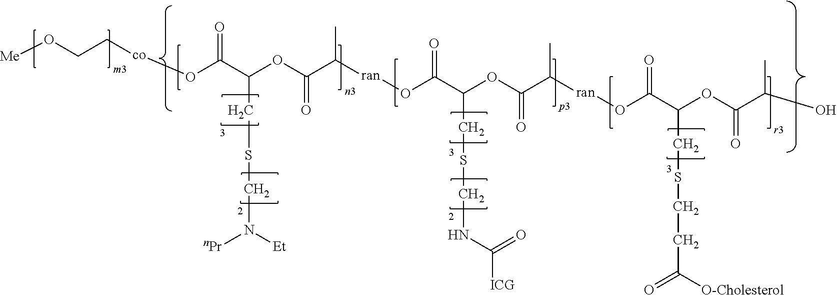
wherein m₃ is in a range of 22-1136, preferably 44-226, n₃ is in a range of 10-500, preferably 30-200, p₃ is in a range of 0.5-5, and r₃ is in a range of 1-200; 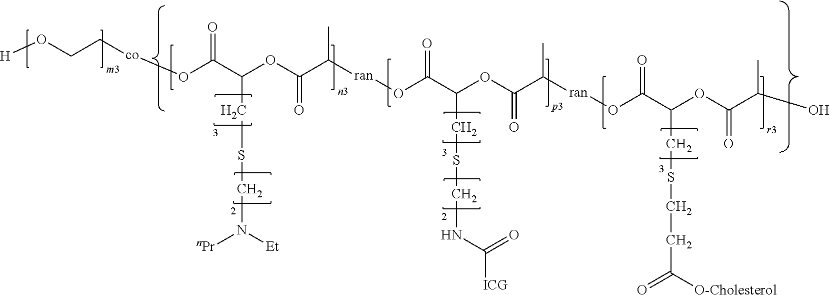
wherein m₃ is in a range of 22-1136, preferably 44-226, n₃ is in a range of 10-500, preferably 30-200, p₃ is in a range of 0.5-5, and r₃ is in a range of 1-200; 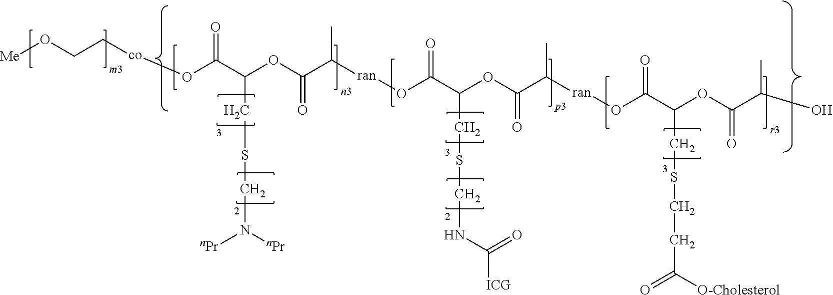
wherein m₃ is in a range of 22-1136, preferably 44-226, n₃ is in a range of 10-500, preferably 30-200, p₃ is in a range of 0.5-5, and r₃ is in a range of 1-200; 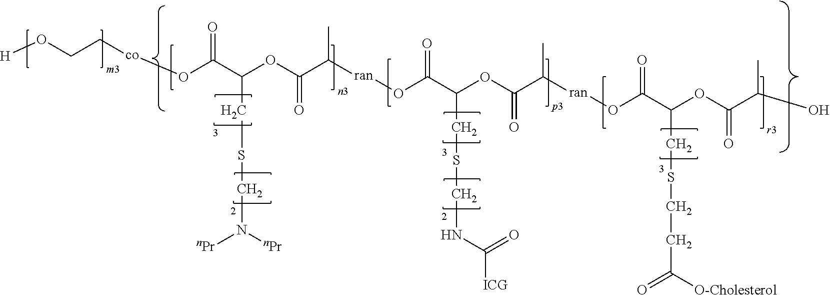
wherein m₃ is in a range of 22-1136, preferably 44-226, n₃ is in a range of 10-500, preferably 30-200, p₃ is in a range of 0.5-5, and r₃ is in a range of 1-200; 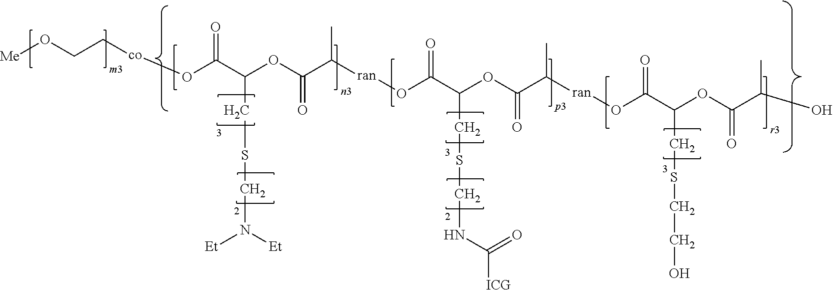
wherein m₃ is in a range of 22-1136, preferably 44-226, n₃ is in a range of 10-500, preferably 30-200, p₃ is in a range of 0.5-5, and r₃ is in a range of 1-200; 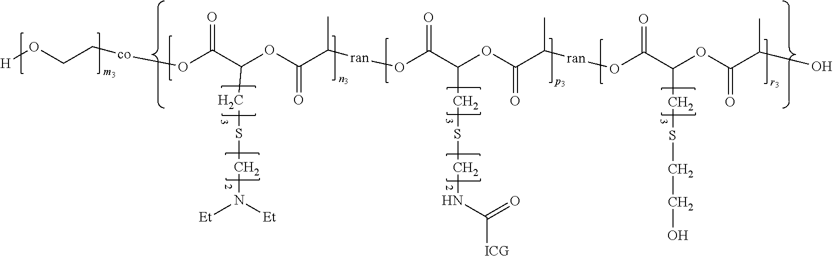
wherein m₃ is in a range of 22-1136, preferably 44-226, n₃ is in a range of 10-500, preferably 30-200, p₃ is in a range of 0.5-5, and r₃ is in a range of 1-200; 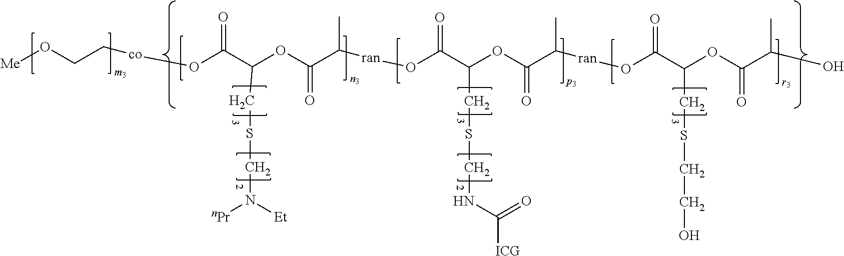
wherein m₃ is in a range of 22-1136, preferably 44-226, n₃ is in a range of 10-500, preferably 30-200, p₃ is in a range of 0.5-5, and r₃ is in a range of 1-200; 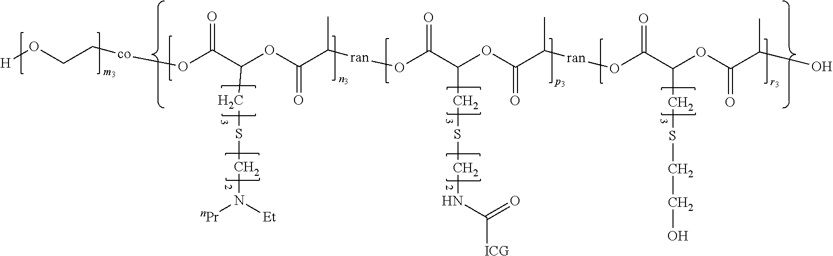
wherein m₃ is in a range of 22-1136, preferably 44-226, n₃ is in a range of 10-500, preferably 30-200, p₃ is in a range of 0.5-5, and r₃ is in a range of 1-200; 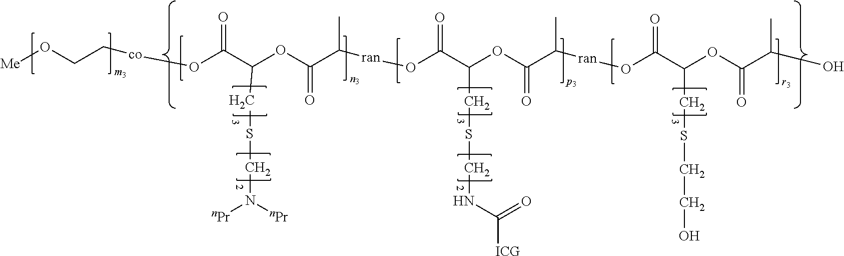
wherein m₃ is in a range of 22-1136, preferably 44-226, n₃ is in a range of 10-500, preferably 30-200, p₃ is in a range of 0.5-5, and r₃ is in a range of 1-200; 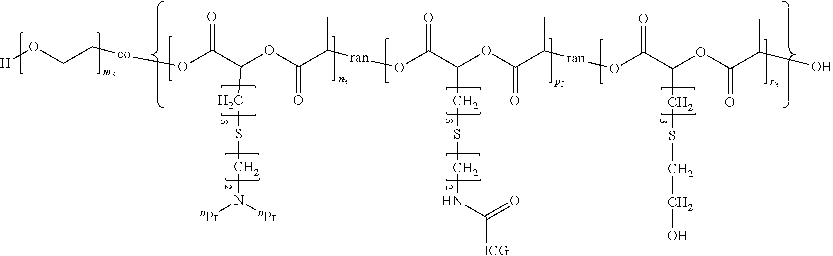
wherein m₃ is in a range of 22-1136, preferably 44-226, n₃ is in a range of 10-500, preferably 30-200, p₃ is in a range of 0.5-5, and r₃ is in a range of 1-200; 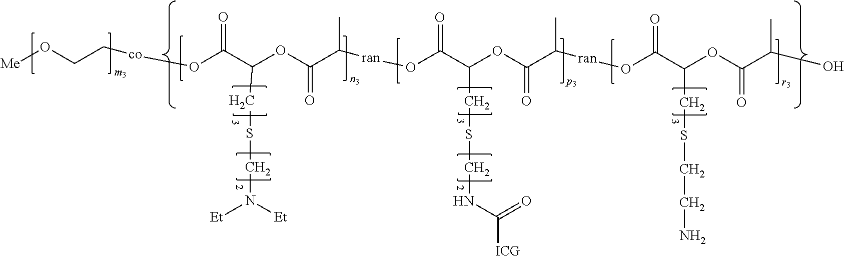
wherein m₃ is in a range of 22-1136, preferably 44-226, n₃ is in a range of 10-500, preferably 30-200, p₃ is in a range of 0.5-5, and r₃ is in a range of 1-200; 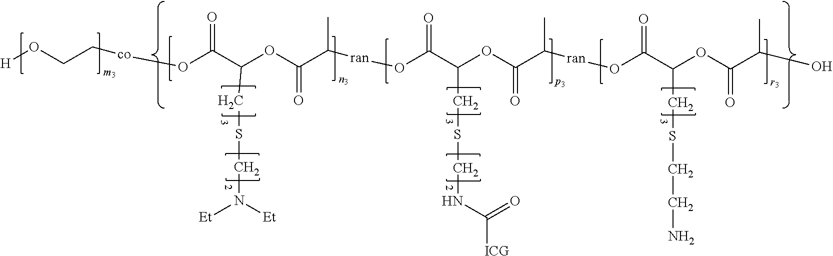
wherein m₃ is in a range of 22-1136, preferably 44-226, n₃ is in a range of 10-500, preferably 30-200, p₃ is in a range of 0.5-5, and r₃ is in a range of 1-200; 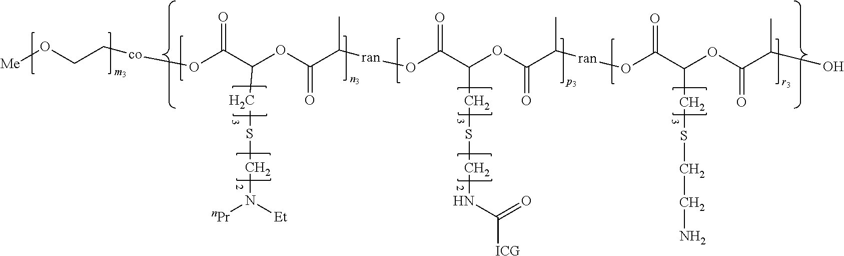
wherein m₃ is in a range of 22-1136, preferably 44-226, n₃ is in a range of 10-500, preferably 30-200, p₃ is in a range of 0.5-5, and r₃ is in a range of 1-200; 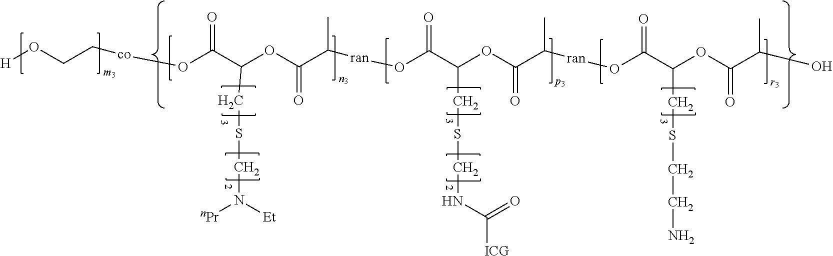
wherein m₃ is in a range of 22-1136, preferably 44-226, n₃ is in a range of 10-500, preferably 30-200, p₃ is in a range of 0.5-5, and r₃ is in a range of 1-200; 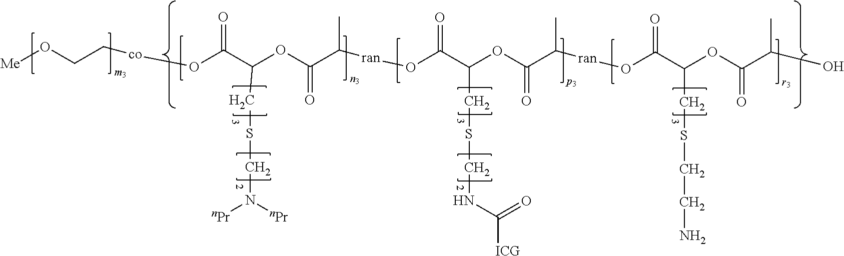
wherein m₃ is in a range of 22-1136, preferably 44-226, n₃ is in a range of 10-500, preferably 30-200, on is in a range of 0.5-5. and r₃ is in a range of 1-200; 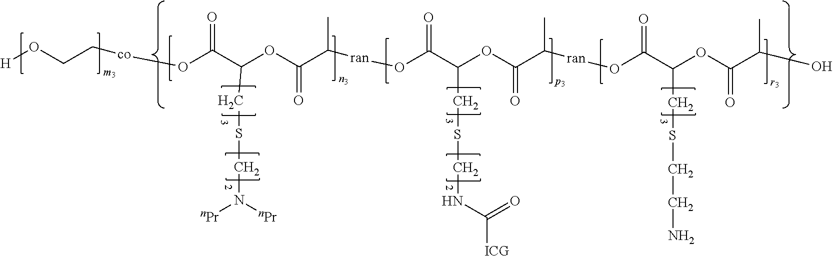
wherein m₃ is in a range of 22-1136, preferably 44-226, n₃ is in a range of 10-500, preferably 30-200, p₃ is in a range of 0.5-5, and r₃ is in a range of 1-200; 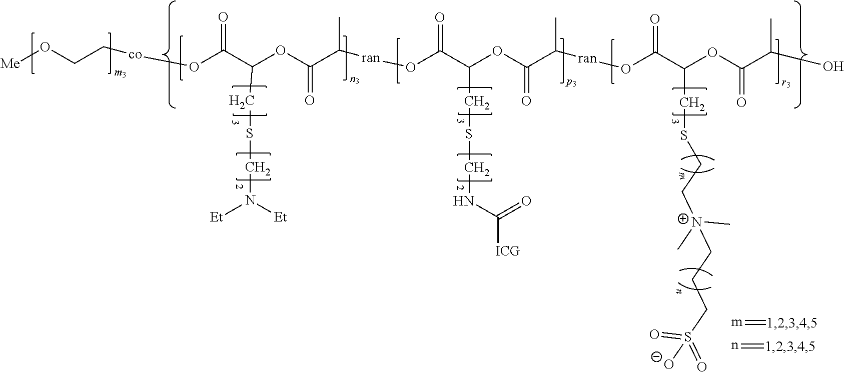
wherein m₃ is in a range of 22-1136, preferably 44-226, n₃ is in a range of 10-500, preferably 30-200, p₃ is in a range of 0.5-5, and r₃ is in a range of 1-200; 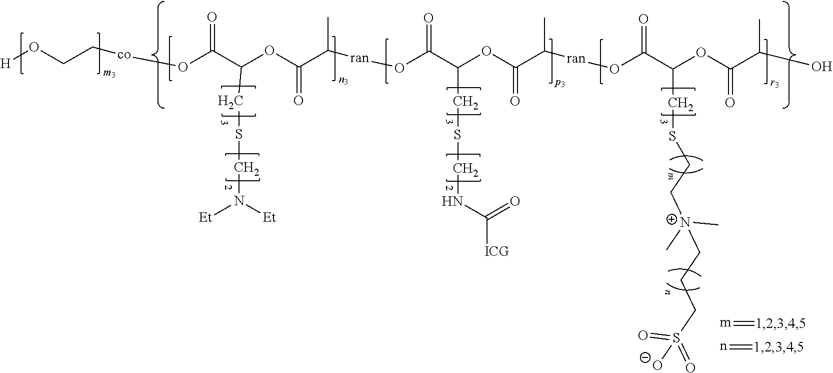
wherein m₃ is in a range of 22-1136, preferably 44-226, n₃ is in a range of 10-500, preferably 30-200, p₃ is in a range of 0.5-5, and r₃ is in a range of 1-200; 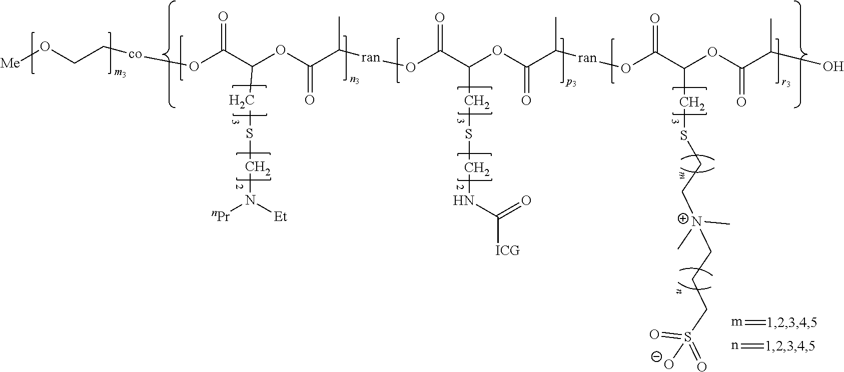
wherein m₃ is in a range of 22-1136, preferably 44-226, n₃ is in a range of 10-500, preferably 30-200, p₃ is in a range of 0.5-5, and r₃ is in a range of 1-200; 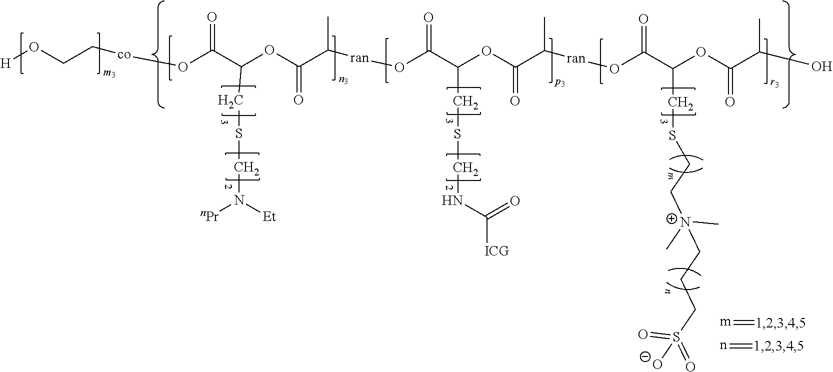
wherein m₃ is in a range of 22-1136, preferably 44-226, n₃ is in a range of 10-500, preferably 30-200, p₃ is in a range of 0.5-5, and r₃ is in a range of 1-200; 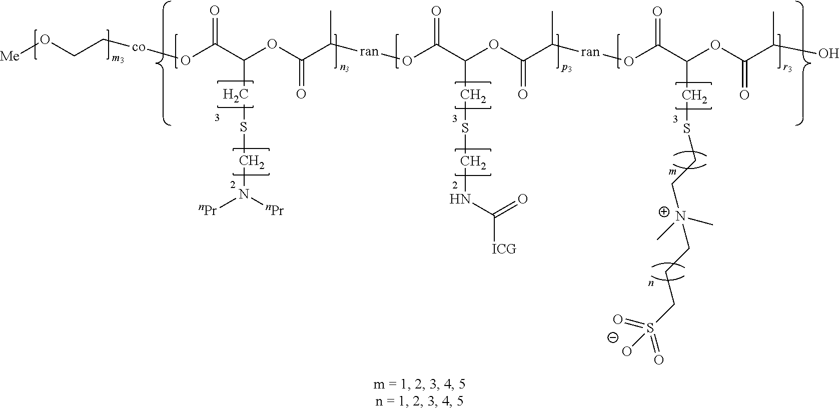
wherein m₃ is in a range of 22-1136, preferably 44-226, n₃ is in a range of 10-500, preferably 30-200, p₃ is in a range of 0.5-5, and r₃ is in a range of 1-200; 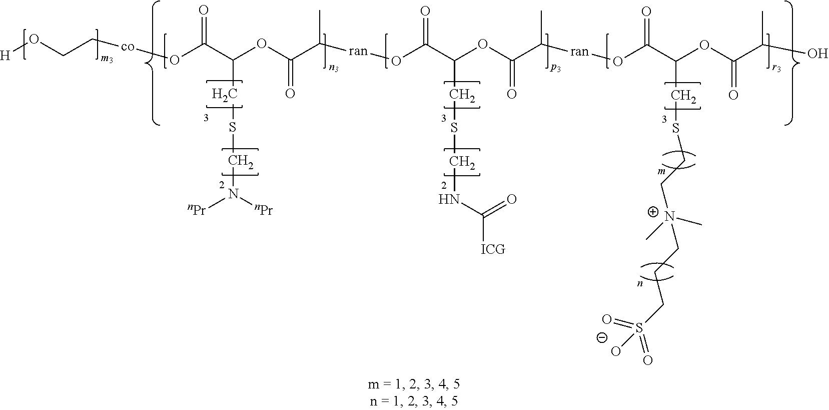
wherein m₃ is in a range of 22-1136, preferably 44-226, n₃ is in a range of 10-500, preferably 30-200, p₃ is in a range of 0.5-5, and r₃ is in a range of 1-200; 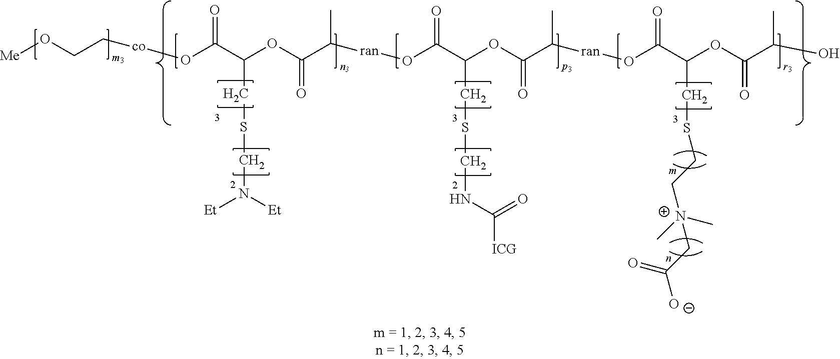
wherein m₃ is in a range of 22-1136, preferably 44-226, n₃ is in a range of 10-500, preferably 30-200, p₃ is in a range of 0.5-5, and r₃ is in a range of 1-200; 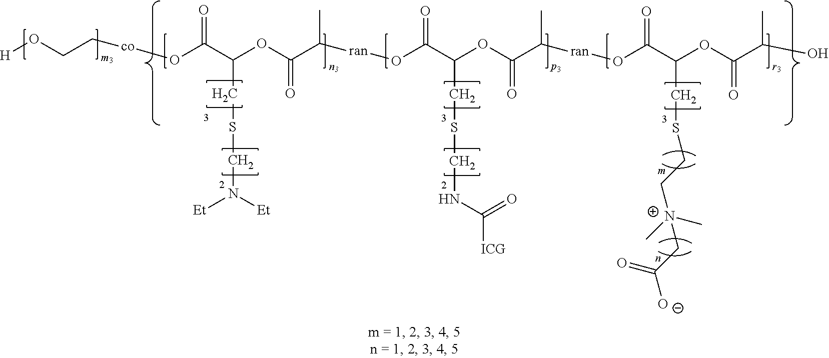
wherein m₃ is in a range of 22-1136, preferably 44-226, n₃ is in a range of 10-500, preferably 30-200, p₃ is in a range of 0.5-5, and r₃ is in a range of 1-200; 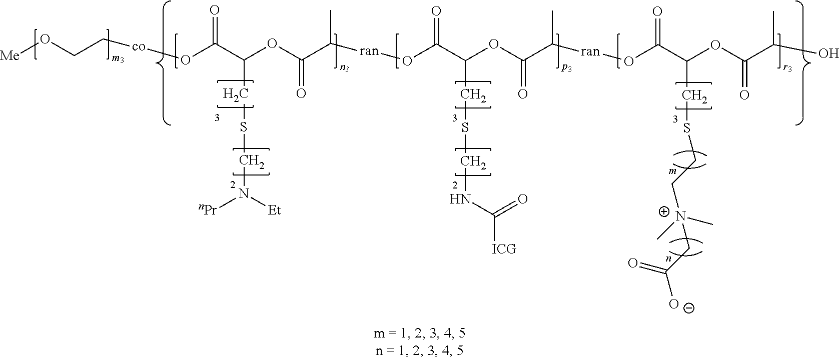
wherein m₃ is in a range of 22-1136, preferably 44-226, n₃ is in a range of 10-500, preferably 30-200, p₃ is in a range of 0.5-5, and r₃ is in a range of 1-200; 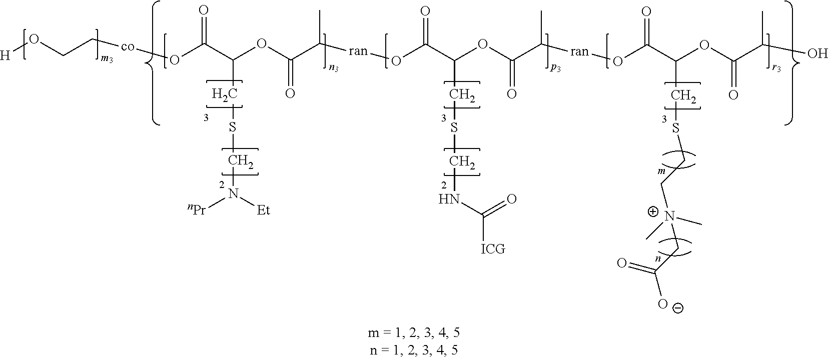
wherein m3 is in a range of 22-1136, preferably 44-226, n₃ is in a range of 10-500, preferably 30-200, p₃ is in a range of 0.5-5, and r₃ is in a range of 1-200; 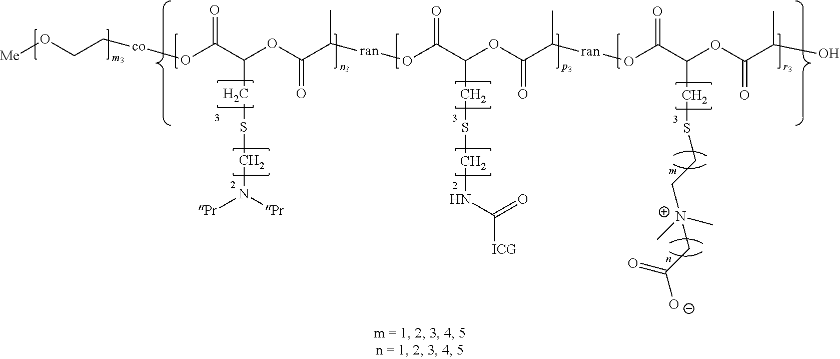
wherein m₃ is in a range of 22-1136, preferably 44-226, n₃ is in a range of 10-500, preferably 30-200, p₃ is in a range of 0.5-5, and r₃ is in a range of 1-200; 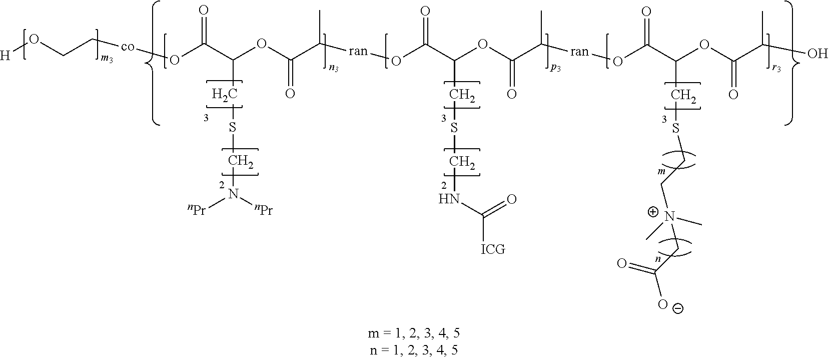
wherein m₃ is in a range of 22-1136, preferably 44-226, n₃ is in a range of 10-500, preferably 30-200, p₃ is in a range of 0.5-5, and r₃ is in a range of 1-200; 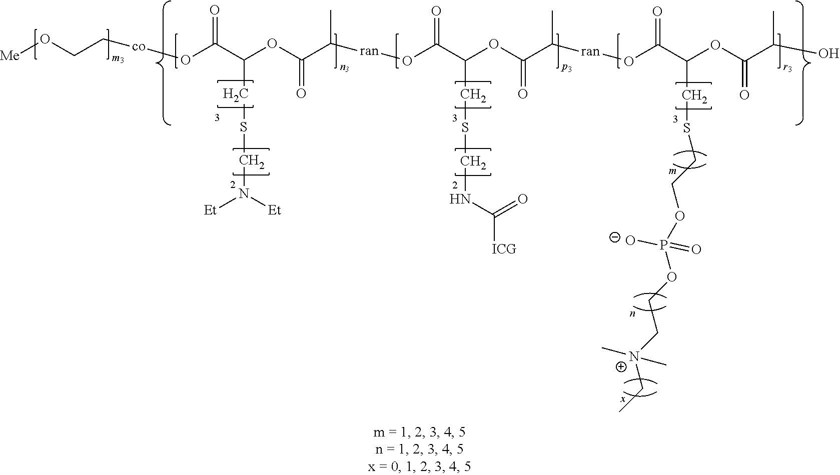
wherein m₃ is in a range of 22-1136, preferably 44-226, n₃ is in a range of 10-500, preferably 30-200, p₃ is in a range of 0.5-5, and r₃ is in a range of 1-200; 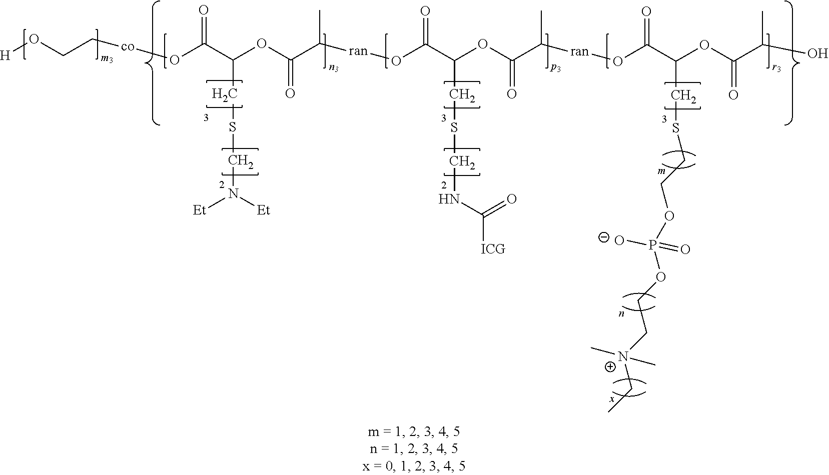
wherein m₃ is in a range of 22-1136, preferably 44-226, n₃ is in a range of 10-500, preferably 30-200, p₃ is in a range of 0.5-5, and r₃ is in a range of 1-200; 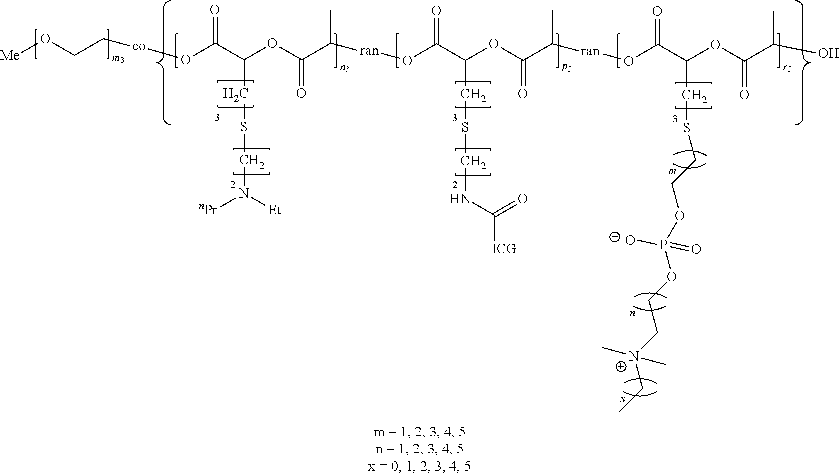
wherein m₃ is in a range of 22-1136, preferably 44-226, n₃ is in a range of 10-500, preferably 30-200, p₃ is in a range of 0.5-5, and r₃ is in a range of 1-200; 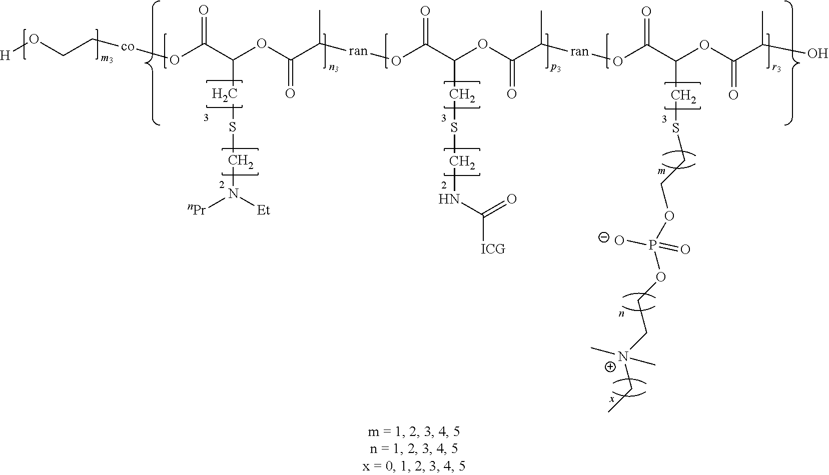
wherein m₃ is in a range of 22-1136, preferably 44-226, n₃ is in a range of 10-500, preferably 30-200, p₃ is in a range of 0.5-5, and r₃ is in a range of 1-200; 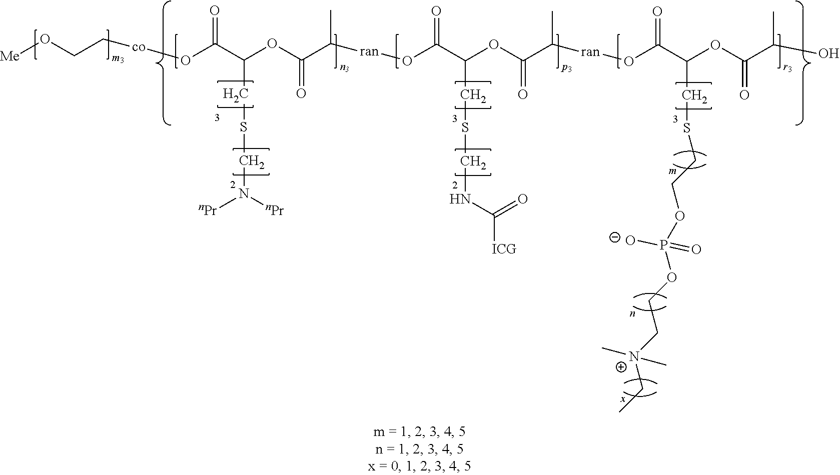
wherein m₃ is in a range of 22-1136, preferably 44-226, n₃ is in a range of 10-500, preferably 30-200, p₃ is in a range of 0.5-5, and r₃ is in a range of 1-200; 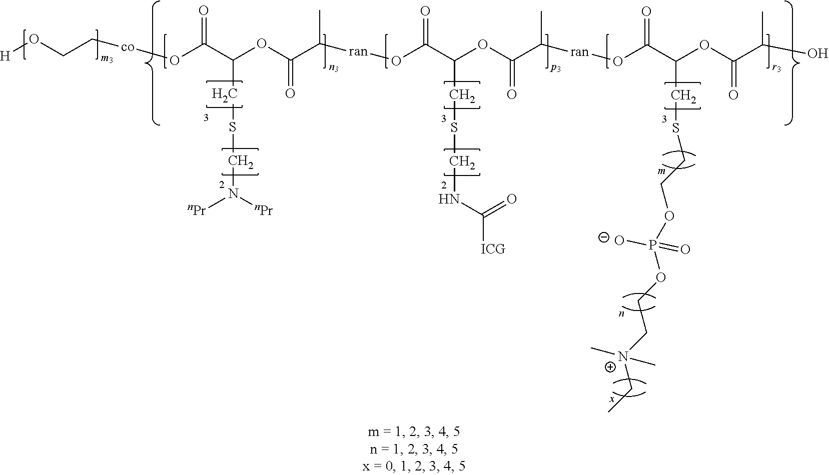
wherein m₃ is in a range of 22-1136, preferably 44-226, n₃ is in a range of 10-500, preferably 30-200, p₃ is in a range of 0.5-5, and r₃ is in a range of 1-200; 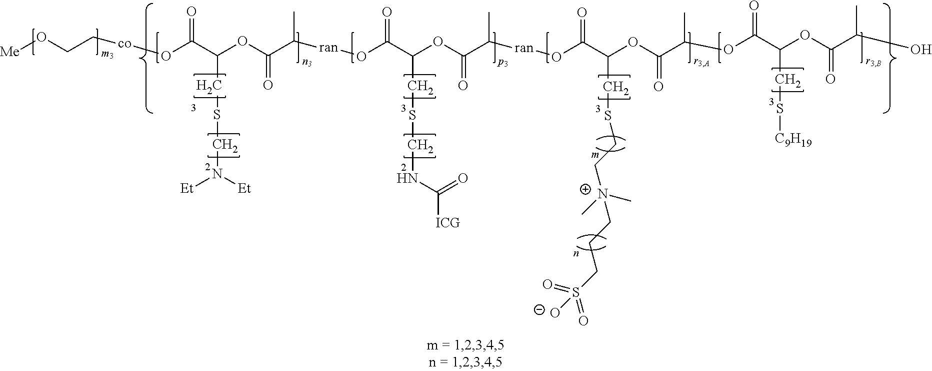
wherein m₃ is in a range of 22˜1136, preferably 44˜226, n₃ is in a range of 10˜500, preferably 30˜200, p₃ is in a range of 0.5˜5, (r_{3,A}+r_{3,B}) is in a range of 1˜200; 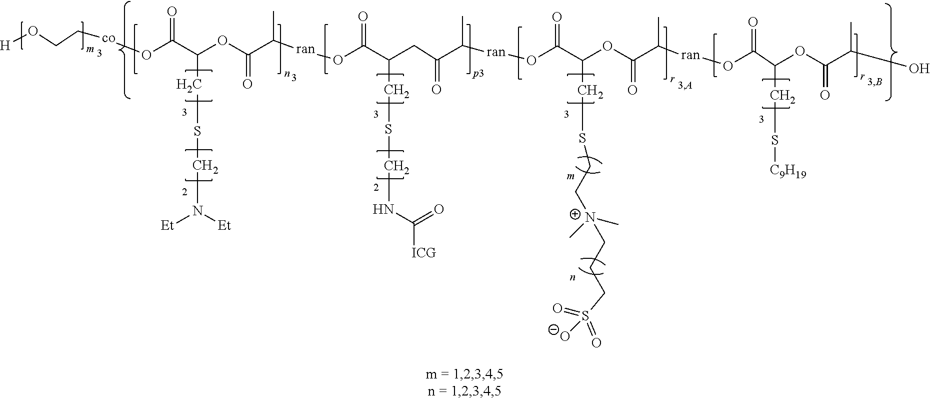
wherein m₃ is in a range of 22˜1136, preferably 44˜226, n₃ is in a range of 10˜500, preferably 30˜200, p₃ is in a range of 0.5˜5, (r_{3,A}+r_{3,B}) is in a range of 1˜200; 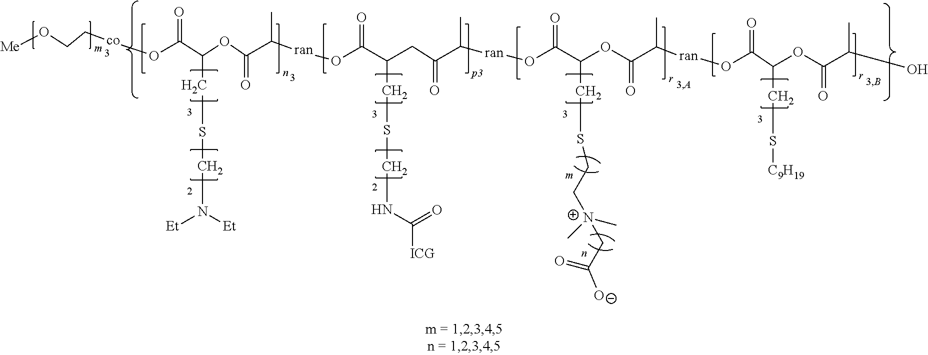
wherein m₃ is in a range of 22˜1136, preferably 44˜226, n₃ is in a range of 10˜500, preferably 30˜200, p₃ is in a range of 0.5˜5, (r_{3,A}+r_{3,B}) is in a range of 1˜200; 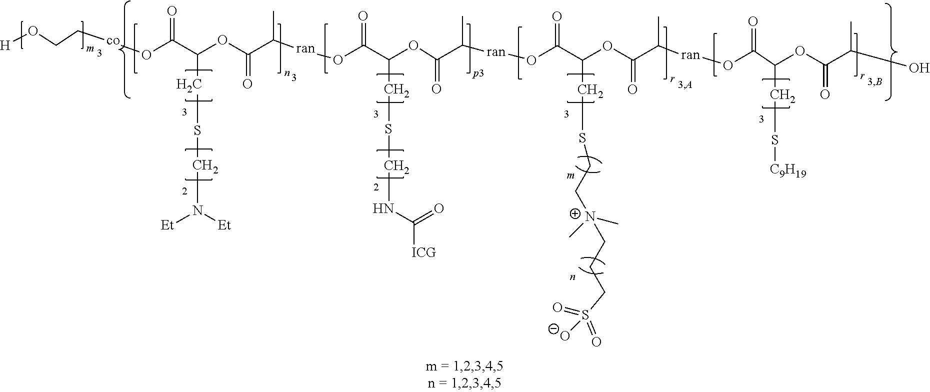
wherein m₃ is in a range of 22˜1136, preferably 44˜226, n₃ is in a range of 10˜500, preferably 30˜200, p₃ is in a range of 0.5˜5, (r_{3,A}+r_{3,B}) is in a range of 1˜200; 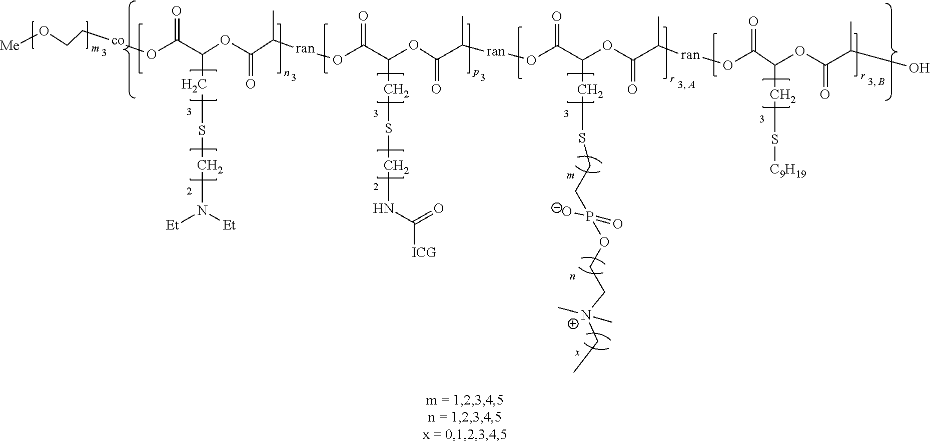
wherein m₃ is in a range of 22˜1136, preferably 44˜226, n₃ is in a range of 10˜500, preferably 30˜200, p₃ is in a range of 0.5˜5, (r_{3,A}+r_{3,B}) is in a range of 1˜200; 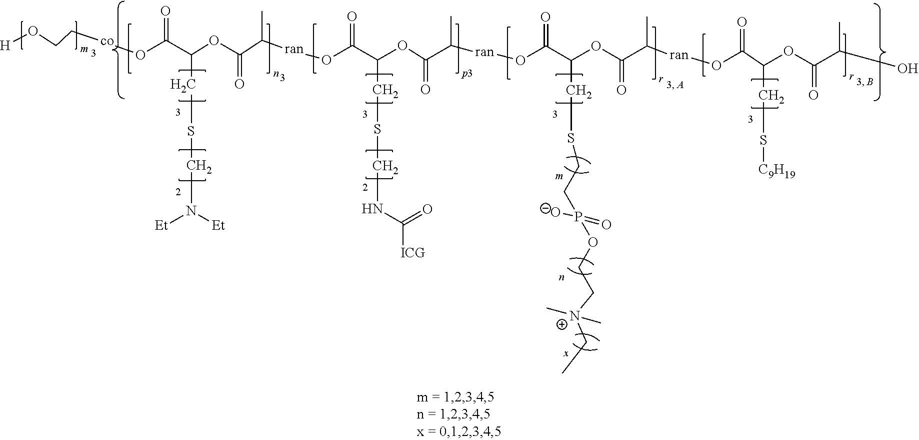
wherein m₃ is in a range of 22˜1136, preferably 44˜226, n₃ is in a range of 10˜500, preferably 30˜200, p₃ is in a range of 0.5˜5, (r_{3,A}+r_{3,B}) is in a range of 1˜200; 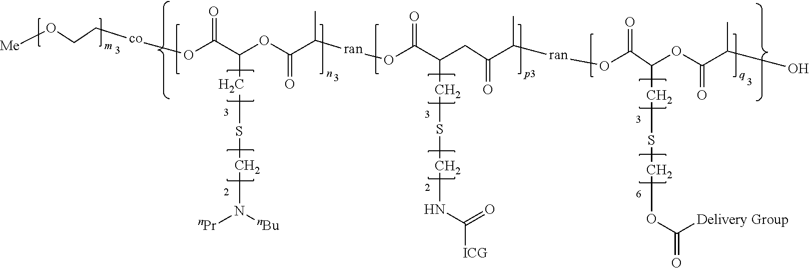
wherein m₃ is in a range of 22˜1136, preferably 44˜226, n₃ is in a range of 10˜500, preferably 30˜200, p₃ is in a range of 0.5˜5, and q₃ is in a range of 0˜500, preferably 10˜200; 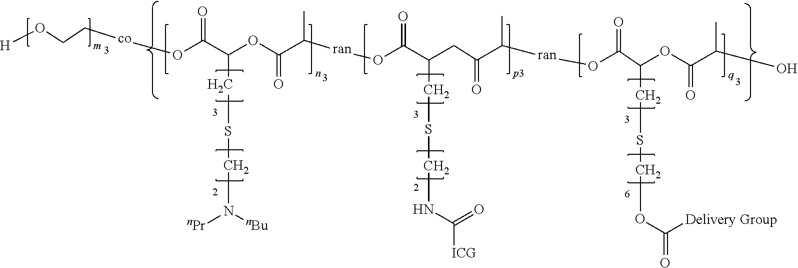
wherein m₃ is in a range of 22-1136, preferably 44-226, n₃ is in a range of 10-500, preferably 30-200, p₃ is in a range of 0.5-5, and q₃ is in a range of 0-500, preferably 10-200; 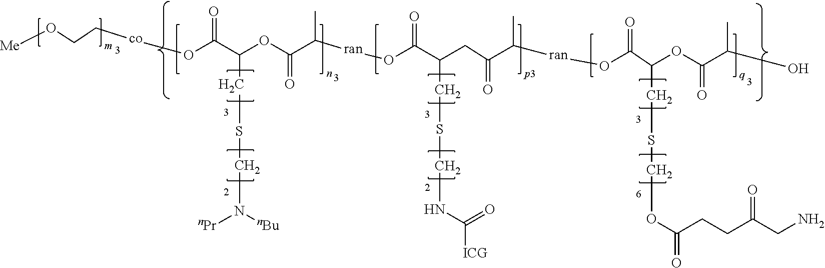
wherein m₃ is in a range of 22-1136, preferably 44-226, n₃ is in a range of 10-500, preferably 30-200, p₃ is in a range of 0.5-5, and q₃ is in a range of 0-500, preferably 10-200; 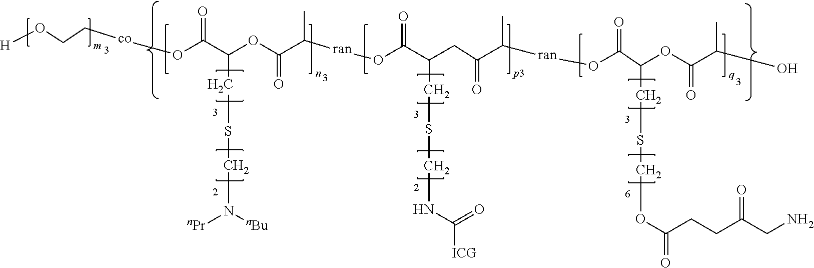
wherein m₃ is in a range of 22-1136, preferably 44-226, n₃ is in a range of 10-500, preferably 30-200, p₃ is in a range of 0.5-5, and q₃ is in a range of 0-500, preferably 10-200; 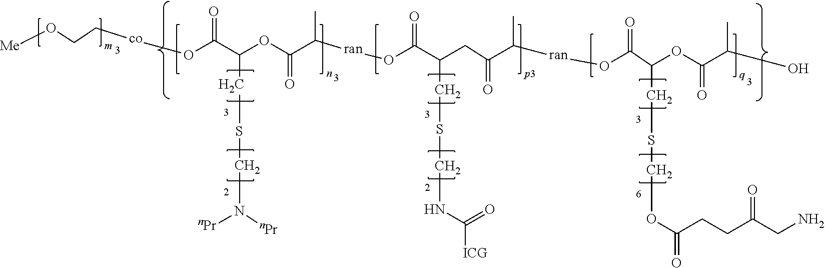
wherein m₃ is in a range of 22-1136, preferably 44-226, n₃ is in a range of 10-500, preferably 30-200, p₃ is in a range of 0.5-5, and q₃ is in a range of 0-500, preferably 10-200; 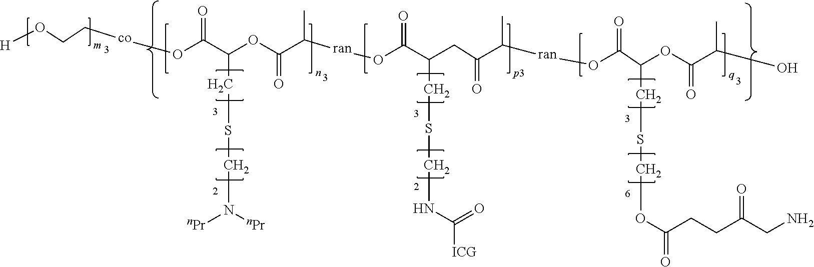
wherein m₃ is in a range of 22-1136, preferably 44-226, n₃ is in a range of 10-500, preferably 30-200, p₃ is in a range of 0.5-5, and q₃ is in a range of 0-500, preferably 10-200; 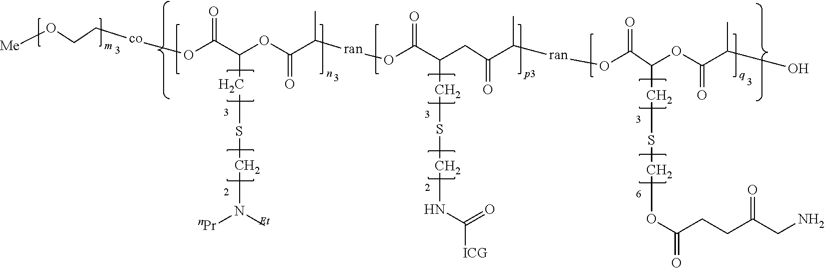
wherein m₃ is in a range of 22-1136, preferably 44-226, n₃ is in a range of 10-500, preferably 30-200, p₃ is in a range of 0.5-5, and q₃ is in a range of 0-500, preferably 10-200; 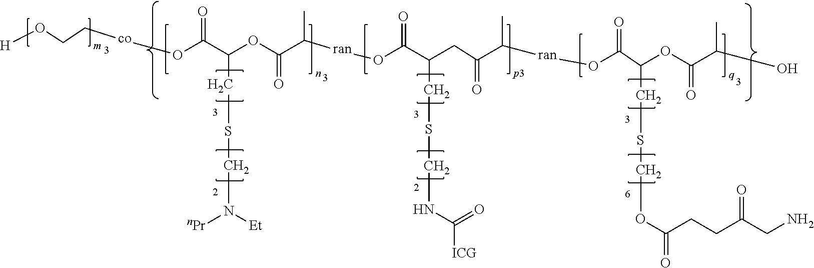
wherein m₃ is in a range of 22-1136, preferably 44-226, n₃ is in a range of 10-500, preferably 30-200, p₃ is in a range of 0.5-5, and q₃ is in a range of 0-500, preferably 10-200; 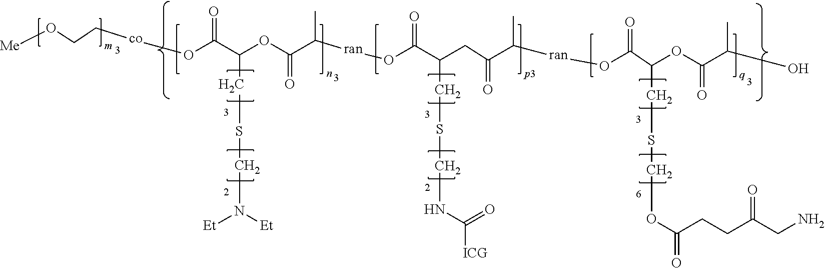
wherein m₃ is in a range of 22-1136, preferably 44-226, n₃ is in a range of 10-500, preferably 30-200, p₃ is in a range of 0.5-5, and q₃ is in a range of 0-500, preferably 10-200; 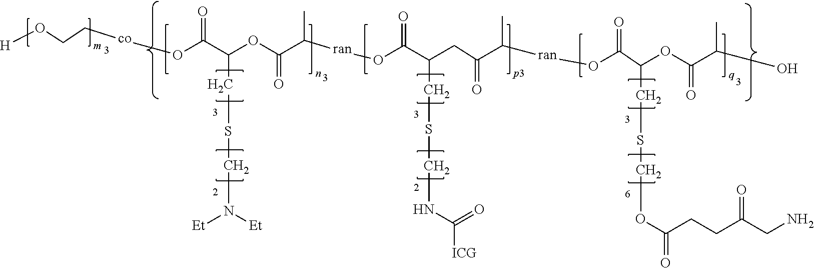
wherein m₃ is in a range of 22-1136, preferably 44-226, n₃ is in a range of 10-500, preferably 30-200, p₃ is in a range of 0.5-5, and q₃ is in a range of 0-500, preferably 10-200.

6. Polymer particles, prepared from the functionalized diblock copolymer according to any one of claims 1 to 5.

7. The polymer particles of claim 6, wherein a particle size of the polymer particles is in a range of 10 to 200 nm;
and/or, the polymer particles are further modified with a targeting group, and the targeting group is selected from the group consisting of monoclonal antibody fragments, small molecule targeting groups, polypeptide molecules, and nucleic acid aptamers;
and/or, the targeting group is modified on at least part of the T-terminal of the functionalized diblock copolymer.

8. The functionalized diblock copolymer according to claim 1, or the polymer particles prepared from the functionalized diblock copolymer, wherein the functionalized diblock copolymer and/or the polymer particles are degradable in vivo.

9. Use of the functionalized diblock copolymer according to claim 1, or the polymer particles prepared from the functionalized diblock copolymer in the preparation of an imaging probe reagent and/or a pharmaceutical preparation, wherein the imaging probe reagent and/or the pharmaceutical preparation preferably has a targeting function, wherein the imaging probe reagent and/or the pharmaceutical preparation is more preferably a targeting imaging probe.

10. A composition, comprising the functionalized diblock copolymer according to claim 1, or the polymer particles prepared from the functionalized diblock copolymer.

11. A method of treating or diagnosing a tumor, wherein the method comprises: administering to an individual an effective amount of the functionalized diblock copolymer according to claim 1, or administering to an individual an effective amount of the polymeric particles prepared from the functionalized diblock copolymer.

12. The method according to claim 11, wherein the functionalized diblock copolymer or the polymeric particles are administered to the individual by administration methods including bladder instillation, uterine perfusion, intestinal perfusion, local administration to the brain after craniotomy, local administration during breast cancer dissection surgery, topical administration during minimally invasive surgery for abdominal tumor.
